SEQUENCE LISTING (47 sequences)

SEQ ID NO: 1-8 - skipped (8 entries)

SEQ ID NO: 9 - protein, 247 aa, Homo sapiens
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 10 - protein, 201 aa, Homo sapiens
MEYHPDLENLDEDGYTQLHFDSQSNTRIAVVSEKGSCAASPPWRLIAVILGILCLVILVIAVVLGTMGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 11-14 - skipped (4 entries)

SEQ ID NO: 15 - protein, 582 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGSGGGSGGGSEFASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGS

SEQ ID NO: 16 - skipped

SEQ ID NO: 17 - protein, 365 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGSGGGSGGGSEFASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSAAAGASHHHHHH

SEQ ID NO: 18 - protein, 368 aa, synthetic construct
QWQLQQSGAELARPGASWKMSCKASGYTFTTYTMHWWKQRPGQGLEWIGYINPSSGYTNYNQKFKDKATLTADKSSSTASMQLSSLTSEDSAWYYCARERAVLVPYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGSGGGSGGGSEFASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSAAAGASHHHHHH

SEQ ID NO: 19 - protein, 213 aa, synthetic construct
QIVLTQSPAVMSASPGEKWTITCTASSSLSYMHWFQQKPGTSPKLWLYSTSILASGVPTRFSGSGSGTSYSLTISRMEAEDAATYYCQQRSSSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 248 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVAAAGASHHHHHHGSGLNDIFEAQKIEWHE

SEQ ID NO: 21 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 247 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVAAAGASHHHHHHGSGLNDIFEAQKIEWHE

SEQ ID NO: 23 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 121 aa, synthetic construct [sequence omitted]

SEQ ID NO: 25 - protein, 106 aa, synthetic construct [sequence omitted]

SEQ ID NO: 26-30 - skipped (5 entries)

SEQ ID NO: 31 - protein, 476 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSGGGGSGGGGSGGGGSGGGGSDIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIEEPKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 450 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 33 - protein, 213 aa, synthetic construct; identical to SEQ ID NO: 21

SEQ ID NO: 34 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 35 - protein, 477 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSGGGGSGGGGSGGGGSGGGGSDIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIEEPKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 476 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSGGGGSGGGGSGGGGSGGGGSDIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIEEPKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 38 - protein, 450 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 477 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSGGGGSGGGGSGGGGSGGGGSDIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIEEPKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41-45 - skipped (5 entries)

SEQ ID NO: 46 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 47 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

(19) United States
(12) Patent Application Publication
(10) Pub. No.: US 2025/0223364 A1
(43) Pub. Date: Jul. 10, 2025

(54) MULTISPECIFIC BINDING PROTEINS THAT BIND DECTIN-1 AND CD20 AND METHODS OF USE THEREOF

(71) Applicant: Dren Bio, Inc., Foster City, CA (US)

(72) Inventors: Nenad TOMASEVIC, Foster City, CA (US); Andrew P. AH YOUNG-CHAPON, South San Francisco, CA (US); Xiaodi DENG, Foster City, CA (US); Sridhar VISWANATHAN, Foster City, CA (US); Panagiotis FOTAKIS, Foster City, CA (US); Ruo Shi SHI, San Mateo, CA (US)

(73) Assignee: Dren Bio, Inc., Foster City, CA (US)

(21) Appl. No.: 18/849,345

(22) PCT Filed: Apr. 3, 2023
(86) PCT No.: PCT/US2023/065289

§ 371 Date: Sep. 20, 2024

Related U.S. Application Data
(60) Provisional application No. 63/327,281, filed on Apr. 4, 2022.

(51) Int. Cl. C07K 16/28; A61K 39/00; A61P 35/00; C12N 9/10; C12N 9/24

(52) U.S. Cl. CPC C07K 16/2851; A61P 35/00; C07K 16/2887; C12N 9/1051; C12N 9/2402; C12Y 204/01144; C12Y 302/01114; A61K 2039/505; C07K 2317/31; C07K 2317/55; C07K 2317/622; C07K 2317/73; C07K 2317/732; C07K 2317/75; C07K 2317/92

(57) ABSTRACT

The present disclosure relates to multispecific (e.g., bispecific) binding proteins that bind human Dectin-1 and human CD20, and methods of use and production related thereto.

### CROSS-REFERENCE TO RELATED APPLICATIONS

This application claims priority to U.S. Provisional Application No. 63/327,281, filed Apr. 4, 2022, the disclosures of which are incorporated herein by reference in their entirety.

### REFERENCE TO AN ELECTRONIC SEQUENCE LISTING

The contents of the electronic sequence listing (186542000840SEQLIST.xml; Size: 33,783 bytes; and Date of Creation: Mar. 30, 2023) are herein incorporated by reference in their entirety.

### FIELD

The present disclosure relates to multispecific (e.g., bispecific) binding proteins that bind human Dectin-1 and human CD20, and methods of use and production related thereto.

### BACKGROUND

Phagocytosis is a major mechanism used to remove pathogens and cell debris. Professional phagocytes, such as monocytes, macrophages, dendritic cells, and granulocytes, specifically recognize and engulf host or foreign agents that are aberrant or cause disease. The engulfed material is destroyed through the endo-lysosomal pathway in the phagocytes. Moreover, dendritic cells and macrophages can present antigens to the cells of the adaptive immune system to further promote the elimination of the disease-causing agents.

Dectin-1 is a C-type lectin receptor that recognizes beta-glucans and promotes anti-fungal phagocytic activities. It is expressed on phagocytes and has been clearly shown to be sufficient for activating phagocytosis. Dectin-1 can be exploited for antibody-targeted phagocytosis and elimination of disease-causing agents.

It would be beneficial to develop targeted removal and degradation of accumulated disease-causing agents without boosting overall phagocytosis. This disclosure provides a solution for the problems and describes other advantages.

All references cited herein, including patent applications, patent publications, and scientific literature, are herein incorporated by reference in their entirety, as if each individual reference were specifically and individually indicated to be incorporated by reference.

### BRIEF SUMMARY

The present disclosure relates to multispecific (e.g., bispecific) binding molecules that bind human Dectin-1, and methods of use and production related thereto. Described herein are methods of targeted phagocytosis to remove disease-causing agents, including host cells/host cell products, microbes or their products, etc., upon administration of multispecific (e.g., bispecific) binding molecules comprising a Dectin-1 binding arm and a second arm that specifically binds to the agent, e.g., CD20. The multispecific (e.g., bispecific) binding molecules allow the phagocyte to engage the target agent and form a synapse between it and promote clustering of Dectin-1 on the phagocyte. This stimulates phagocytosis of the target agent and at the same time cytokine secretion via the Dectin-1/Syk/NfkB pathway by the phagocyte. Moreover, antigens from the engulfed material are presented on the surface of dendritic cells/macrophages to boost an adaptive immune response against the disease-causing agent. Overall, it is thought that the Dectin-1 agonistic, multispecific (e.g., bispecific) binding molecules promote immune stimulation, targeted phagocytosis, and neo-antigen presentation/activation of the adaptive immune system to eliminate the disease-causing agent.

As such, the present disclosure describes, inter alia, the generation and functional characterization of an agonistic anti-human Dectin-1 antibody that exhibits high affinity binding to Dectin-1 and can promote immune stimulation. Further described is the generation of bispecific antibody formats including the anti-human Dectin-1 antibody with antibodies targeting antigens on disease-causing agents, with data supporting target engagement, immune stimulation, phagocytosis, and antigen presentation.

In some embodiments, provided herein is a multispecific binding protein, comprising a first polypeptide chain comprising the amino acid sequence QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLWCLVKGFYPSDIAVEWESN GQPENNYKTTPPVLDSDGSFFLYSKLTVDKSRWQQGNVFSCSVMHEALHNHYTQKSLS LSPG (SEQ ID NO:31) or QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLWCLVKGFYPSDIAVEWESN GQPENNYKTTPPVLDSDGSFFLYSKLTVDKSRWQQGNVFSCSVMHEALHNHYTQKSLS LSPGK (SEQ ID NO:35); a second polypeptide chain comprising the amino acid sequence QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLSCAVKGFYPSDIAVEWESNGQPENNYKTTPPVLDSDGSFFLVS KLTVDKSRWQQGNVFSCSVMHEALHNHYTQKSLSLSPG (SEQ ID NO:32) or QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLSCAVKGFYPSDIAVEWESNGQPENNYKTTPPVLDSDGSFFLVS KLTVDKSRWQQGNVFSCSVMHEALHNHYTQKSLSLSPGK (SEQ ID NO:36); and a third polypeptide chain comprising the amino acid sequence QIVLSQSPAILSASPGEKVTMTCRASSSVSYIHWFQQKPGSSPKPWIYATSNLASGVPVRF SGSGSGTSYSLTISRVEAEDAATYYCQQWTSNPPTFGGGTKLEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO:33). In some embodiments, the first polypeptide chain comprises the amino acid sequence of SEQ ID NO:31, the second polypeptide chain comprises the amino acid sequence of SEQ ID NO:32, and the third polypeptide chain comprises the amino acid sequence of SEQ ID NO:33. In some embodiments, the first polypeptide chain comprises the amino acid sequence of SEQ ID NO:35, the second polypeptide chain comprises the amino acid sequence of SEQ ID NO:36, and the third polypeptide chain comprises the amino acid sequence of SEQ ID NO:33. In some embodiments, provided herein is a composition comprising a mixture of multispecific binding protein species, wherein each species comprises a first polypeptide chain that comprises the amino acid sequence of SEQ ID NO:31 or SEQ ID NO:35, a second polypeptide chain that comprises the amino acid sequence of SEQ ID NO:32 or SEQ ID NO:36, and a third polypeptide chain that comprises the amino acid sequence of SEQ ID NO:33.

In some embodiments, provided herein is a multispecific binding protein, comprising a first polypeptide chain comprising the amino acid sequence QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLSCAVKGFYPSDIAVEWESN GQPENNYKTTPPVLDSDGSFFLVSKLTVDKSRWQQGNVFSCSVMHEALHNHYTQKSLS LSPG (SEQ ID NO:37) or QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLSCAVKGFYPSDIAVEWESN GQPENNYKTTPPVLDSDGSFFLVSKLTVDKSRWQQGNVFSCSVMHEALHNHYTQKSLS LSPGK (SEQ ID NO:39); a second polypeptide chain comprising the amino acid sequence QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLWCLVKGFYPSDIAVEWESNGQPENNYKTTPPVLDSDGSFFLY SKLTVDKSRWQQGNVFSCSVMHEALHNHYTQKSLSLSPG (SEQ ID NO:38) or QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLWCLVKGFYPSDIAVEWESNGQPENNYKTTPPVLDSDGSFFLY SKLTVDKSRWQQGNVFSCSVMHEALHNHYTQKSLSLSPGK (SEQ ID NO:40); and a third polypeptide chain comprising the amino acid sequence QIVLSQSPAILSASPGEKVTMTCRASSSVSYIHWFQQKPGSSPKPWIYATSNLASGVPVRF SGSGSGTSYSLTISRVEAEDAATYYCQQWTSNPPTFGGGTKLEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO:33). In some embodiments, the first polypeptide chain comprises the amino acid sequence of SEQ ID NO:37, the second polypeptide chain comprises the amino acid sequence of SEQ ID NO:38, and the third polypeptide chain comprises the amino acid sequence of SEQ ID NO:33. In some embodiments, the first polypeptide chain comprises the amino acid sequence of SEQ ID NO:39, the second polypeptide chain comprises the amino acid sequence of SEQ ID NO:40, and the third polypeptide chain comprises the amino acid sequence of SEQ ID NO:33. In some embodiments, provided herein is a composition comprising a mixture of multispecific binding protein species, wherein each species comprises a first polypeptide chain that comprises the amino acid sequence of SEQ ID NO:37 or SEQ ID NO:39, a second polypeptide chain that comprises the amino acid sequence of SEQ ID NO:38 or SEQ ID NO:40, and a third polypeptide chain that comprises the amino acid sequence of SEQ ID NO:33.

In some embodiments, the first, second, and third polypeptide chains are associated in a multispecific binding protein comprising a first antigen binding domain that binds to human Dectin-1 and a second antigen binding domain that binds to human CD20.

In some embodiments, at least one of the first and second polypeptide chains is/are non-fucosylated. In some embodiments, both of the first and second polypeptide chains are non-fucosylated. In some embodiments, the first antigen-binding domain: binds to human Dectin-1 expressed on the surface of a macrophage, monocyte, dendritic cell, or granulocyte; binds to human Dectin-1 expressed on the surface of a cell with an EC50 of less than 2 nM; is capable of binding human or cynomolgus Dectin-1; and/or does not compete with a native ligand of human Dectin-1. In some embodiments, the second antigen binding domain binds to human CD20 expressed on the surface of a B cell.

In some embodiments, provided herein is a polynucleotide encoding the multispecific binding protein of any one of the above embodiments. In some embodiments, provided herein is a vector (e.g., an expression vector) comprising the polynucleotide of any one of the above embodiments.

In some embodiments, provided herein is a host cell (e.g., an isolated host cell or cell line) comprising the polynucleotide or vector of any one of the above embodiments. In some embodiments, the host cell is a yeast, insect, plant, or prokaryotic cell. In some embodiments, the host cell is a mammalian cell. In some embodiments, the mammalian cell is a Chinese hamster ovary (CHO) cell. In some embodiments, the host cell comprises an alpha1,6-fucosyltransferase (Fut8) or alpha-1,3-mannosyl-glycoprotein 2-beta-N-acetylglucosaminyltranferase (MGAT1) knockout. In some embodiments, the host cell overexpresses β1,4-N-acetylglucosaminyltransferase III (GnT-III). In some embodiments, the host cell further overexpresses Golgi μ-mannosidase II (ManII).

In some embodiments, provided herein is a method of producing a multispecific binding protein, comprising culturing the host cell of any one of the above embodiments under conditions suitable for production of the multispecific binding protein. In some embodiments, the method further comprises recovering the multispecific binding protein. In some embodiments, prior to production of the antibody or multispecific binding protein, the host cell is treated with kifunensine.

In some embodiments, provided herein is a pharmaceutical composition comprising the multispecific binding protein of any one of the above embodiments and a pharmaceutically acceptable carrier. In some embodiments, the composition comprises a mixture of multispecific binding protein species, wherein each species comprises a first polypeptide chain that comprises the amino acid sequence of SEQ ID NO:31 or SEQ ID NO:35, a second polypeptide chain that comprises the amino acid sequence of SEQ ID NO:32 or SEQ ID NO:36, and a third polypeptide chain that comprises the amino acid sequence of SEQ ID NO:33. In some embodiments, the first polypeptide chain comprises the amino acid sequence of SEQ ID NO:31, the second polypeptide chain comprises the amino acid sequence of SEQ ID NO:32, and the third polypeptide chain comprises the amino acid sequence of SEQ ID NO:33. In some embodiments, the first polypeptide chain comprises the amino acid sequence of SEQ ID NO:35, the second polypeptide chain comprises the amino acid sequence of SEQ ID NO:36, and the third polypeptide chain comprises the amino acid sequence of SEQ ID NO:33. In some embodiments, the composition comprises a mixture of multispecific binding protein species, wherein each species comprises a first polypeptide chain that comprises the amino acid sequence of SEQ ID NO:37 or SEQ ID NO: 39, a second polypeptide chain that comprises the amino acid sequence of SEQ ID NO:38 or SEQ ID NO:40, and a third polypeptide chain that comprises the amino acid sequence of SEQ ID NO: 33. In some embodiments, the first polypeptide chain comprises the amino acid sequence of SEQ ID NO:37, the second polypeptide chain comprises the amino acid sequence of SEQ ID NO: 38, and the third polypeptide chain comprises the amino acid sequence of SEQ ID NO:33. In some embodiments, the first polypeptide chain comprises the amino acid sequence of SEQ ID NO: 39, the second polypeptide chain comprises the amino acid sequence of SEQ ID NO:40, and the third polypeptide chain comprises the amino acid sequence of SEQ ID NO:33.

In some embodiments, provided herein is a method of treating a disease or disorder, comprising administering an effective amount of the multispecific binding protein or composition of any one of the above embodiments to an individual in need thereof. In some embodiments, the disease or disorder is a B cell-mediated disease or disorder. In some embodiments, the individual has or has been diagnosed with a B cell-mediated disease or disorder. In some embodiments, the B cell-mediated disease or disorder is cancer. In some embodiments, the cancer is a B cell-mediated cancer or is characterized by a B cell malignancy. In some embodiments, the cancer is non-Hodgkin's lymphoma or chronic lymphocytic leukemia. In some embodiments, the B cell-mediated disease or disorder is an autoimmune disease or disorder. In some embodiments, the autoimmune disease or disorder is rheumatoid arthritis, systemic lupus erythematosus (SLE), multiple sclerosis, and Wegener's granulomatosis. In some embodiments, the individual is a human.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIGS. 1A-1C show the binding analysis of the anti-human Dectin-1 antibody (clone 2M24) in human and monkey monocytes derived from peripheral blood mononuclear cells (PBMC) by flow cytometry. Single, live and CD14+ cells were gated to identify monocytes. The cells were incubated with 2M24 anti-Dectin-1 primary antibody or a mIgG1 isotype control antibody, followed by incubation with a fluorescent anti-mouse secondary antibody. The primary antibodies were used in a serial dose titration. FIG. 1A shows the binding analysis for anti-human Dectin-1 clone 2M24 in human monocytes. FIG. 1B shows the binding analysis for anti-human Dectin-1 clone 2M24 antibody in cynomolgus monocytes. FIG. 1C depicts a comparison of binding to human monocytes, HEK cells overexpressing human Dectin-1 and cynomolgus monocytes between the 2M24 clone and other Dectin-1 antibodies identified from the ATX-Gx Alloy transgenic mice immunization as well as commercial anti-Dectin-1 antibodies. Anti-human Dectin-1 clone 2M24 antibody demonstrated high affinity to both human and cynomolgus monkey Dectin-1 expressed in monocytes, and exhibited superior affinity as compared to other anti-Dectin-1 antibodies, including commercial antibodies.

FIGS. 2A-2B show the phagocytosis of pHrodo-labeled polystyrene anti-mouse Fc IgG beads conjugated with anti-Dectin-1 antibody 2M24 or isotype control antibody by HEK-Blue hDectin-1a cells and human monocytes. Polystyrene anti-mouse Fc IgG beads (˜3.4 μm) were labeled with a pH-sensitive fluorescent dye (pHrodo Red) and conjugated with Dectin-1 antibody 2M24 or isotype control. The beads were then incubated with cultured HEK-Blue hDectin-1a cells or human monocytes at a ratio of 1:2 (cells:beads). HEK-Blue hDectin-1a cells were labeled with the cell-permeant dye Calcein AM. The phagocytosis of the beads was monitored by IncuCyte live cell imaging. Phagocytosis was quantified using the IncuCyte analysis software and expressed as overlap of red object count (pHrodo) to calcein-positive cells. FIG. 2A shows the phagocytosis of beads over 2.5 hours in HEK-Blue hDectin-1a cells (top) and representative images of pHrodo positive cells at 2.5 hours of phagocytosis (bottom). FIG. 2B shows the phagocytosis of beads over 4 hours in human monocytes (top), as well as representative images of pHrodo positive cells at 2.5 hours of phagocytosis (bottom). In the representative images, engulfed beads fluoresce brightly in phagosomes.

FIGS. 3A-3B show the binding of the fully human 2M24 anti-Dectin-1 antibody (hIgG4) or isotype control antibody in HEK-Blue hDectin-1a cells and primary human monocytes. FIG. 3A shows the binding analysis of the fully human 2M24 anti-Dectin-1 antibody to HEK cells, while FIG. 3B shows the binding to primary human monocytes. The primary antibodies were used in a serial dose titration followed by a fluorescent secondary antibody against the primary antibody. The fully human 2M24 anti-Dectin-1 hIgG4 antibody bound with high affinity to Dectin-1 expressing cells.

FIG. 4 shows the targeted phagocytosis of pHrodo-labeled polystyrene biotin beads conjugated with the fully human 2M24 anti-Dectin-1 antibody (hIgG4) or isotype control antibody by Dectin-1 expressing cells. Polystyrene biotin beads were labeled with pHrodo Red and conjugated via streptavidin to anti-Dectin-1 antibody 2M24 or an isotype control. The conjugated beads were mixed with cells at a ratio of 1:3, and phagocytosis of the beads was monitored by IncuCyte live cell imaging. The phagocytosis of phrodo-biotin beads conjugated to streptavidin 2M24 anti-Dectin-1 hIgG4 antibody is shown for HEK-Blue hDectin-1a cells (top left), human monocytes (top right) and human macrophages (bottom). The fully human 2M24 anti-Dectin-1 antibody (hIgG4) promoted phagocytosis in Dectin-1 expressing cells.

FIGS. 5A-5B show the results of a secreted alkaline phosphatase reporter assay of Dectin-1 in HEK-Blue hDectin-1a cells. FIG. 5A shows the results for a secreted alkaline phosphatase assay performed using immobilized fully human 2M24 anti-Dectin-1 antibody. The fully human 2M24 (hIgG4) anti-Dectin-1 antibody or an isotype control antibody were immobilized overnight in U-bottomed polypropylene microtiter plates at quantities ranging from 0.1-10 μg per well, followed by culture of HEK-Blue hDectin-1a cells for 22 hours and evaluation of alkaline phosphatase secretion at OD 630 nm in the supernatant. FIG. 5B shows the results for a secreted alkaline phosphatase assay performed using bead-conjugated fully human 2M24 anti-Dectin-1 antibody. Biotin beads of 3, 10 and 16.5 μm in size were conjugated to streptavidin 2M24 (hIgG4) anti-Dectin-1 antibody. Antibody-conjugated beads were mixed with HEK-Blue hDectin-1a cells for 22 hours, and the supernatant was evaluated for alkaline phosphatase secretion at OD 630 nm. Bars represent mean±s.d.; n=2 replicates. The 2M24 (hIgG4) anti-Dectin-1 antibody induced alkaline phosphatase secretion in HEK-Blue hDectin-1a cells both in an immobilized form and conjugated to beads.

FIGS. 6A-6B show the cytokine secretion by human primary macrophages stimulated with anti-Dectin-1 (15E2) antibody in solution. Primary human macrophages and primary monocytes were stimulated with 10 μg/ml of the 15E2 anti-Dectin-1 antibody or isotype antibody in solution for 24 hours, and secretion of TNFa and IL6 was assessed by ELISA analysis of the supernatant. Zymosan was used as positive controls for cytokine secretion. Bars represent mean±s.d.; n=2 replicates FIG. 6A shows the results for primary human monocytes stimulated with soluble 15E2 anti-Dectin-1 antibody, while FIG. 6B shows the results for stimulated primary human macrophages. Soluble 15E2 anti-Dectin-1 antibody did not induce cytokine secretion in primary human monocytes and macrophages.

FIGS. 7A-7B show the cytokine secretion by human primary monocytes and PBMCs stimulated with immobilized 2M24 or 15E2 anti-Dectin-1 antibody. The anti-Dectin-1 antibodies or isotype control antibodies were immobilized overnight in U-bottomed polypropylene microtiter plates at 10 μg per well, followed by culture of human monocytes or human PBMCs for 24 hours. The secretion of TNFa, IL6 and IFNg was evaluated by ELISA analysis of the supernatant. FIG. 7A shows the cytokine secretion by human monocytes following stimulation with immobilized anti-Dectin-1 antibodies, while FIG. 7B shows the cytokine secretion by cultured human PBMCs after stimulation. Bars represent mean±s.d.; n=2 replicates. The 2M24 anti-Dectin-1 antibody induced cytokine secretion in both primary human monocytes and PBMCs and exhibited superior immune stimulation to the 15E2 Dectin-1 agonistic antibody

FIG. 8 shows the results of a competition assay performed using the 12M4 anti-Dectin-1 antibody clone and natural ligands for Dectin-1. HEK-Blue hDectin-1a cells were incubated in a ⅓ serial dose titration of 2M24 (hIgG4) anti-Dectin-1 antibody or the 15E2, 259931, GE2 anti-Dectin-1 commercial antibodies starting at 300 nM and in the presence of 8 ug/ml of biotin-laminarin for 30 minutes on ice. Binding of laminarin to Dectin-1 was assessed by flow cytometry using Streptavidin-Alexa fluor 647. The 2M24 (hIgG4) anti-Dectin-1 antibody did not compete with natural ligand for binding to Dectin-1.

FIG. 9 depicts a summary of the functional characterization of the 2M24 and 15E2 anti-Dectin-1 antibodies.

FIGS. 10A-10B show a schematic illustration of bispecific antibody generation by click chemistry. FIG. 10A depicts the differential labeling of antibodies with MTA or FOL reagents FIG. 10B depicts the covalent crosslinking of antibodies via specific MTA-FOL interactions.

FIG. 11 illustrates the potential modes of activity deployed by anti-Dectin-1 agonistic bispecific antibodies to eliminate target cancer cells. These include immune stimulation, phagocytosis, neo-antigen presentation and activation of T and B lymphocytes of the adaptive immune system.

FIGS. 12A-12B show the characterization of click chemistry-conjugated bispecifics comprising anti-Dectin-1 (clone 2M24) and anti-hCD70 arms. FIG. 12A shows an SDS-PAGE analysis of covalently conjugated antibody pairs (2M24/anti-hCD20, 2M24/anti-hCD70, and isotype controls) under non-reducing and reducing conditions. FIG. 12B shows a flow cytometry-based characterization of bispecific (2M24/anti-hCD70 or isotype control) binding to Dectin-1-expressing HEK293 cells (top left) and two renal carcinoma cell lines-A498 (top right) and 786-0 (bottom left). FIG. 12B also depicts the EC50 concentration (nM) based on a non-linear regression fitting (bottom right). Anti-Dectin-1/anti-hCD70 bispecific binds Dectin-1- or CD70-expressing cells with an affinity of 1.8 nM or 12.34 nM, respectively.

FIG. 13 shows coupling of Dectin-1-expressing HEK293 cell line and A498 renal carcinoma cell line induced by 2M24/anti-hCD70 bispecific. Shown are a flow cytometry analysis of co-cultures of HEK293 cells (labeled with calcein green) and A498 cells (labeled with calcein red) in the presence of 2M24/anti-hCD70 bispecific or isotype control (left). Coupling of HEK293 and A498 cells is indicated by a double-positive signal (green+red+, square box). Also shown is coupling efficiency, which is quantified as the percentage of total target cells (A498) that forms doublets with HEK293 cells (right). Bars represent mean±s.d.; n=3 replicates. The 2M24/anti-hCD70 bispecific antibody induced coupling of Dectin-1-expressing HEK293 cell line and A498 renal carcinoma cell line

FIGS. 14A-14B shows the coupling of Dectin-1-expressing cells and B cells induced by anti-Dectin-1/anti-hCD20 bispecific antibody. FIG. 14A shows the coupling of Dectin-1-expressing HEK293 cells and B cells induced by anti-Dectin-1/anti-hCD20 bispecific antibody. Shown are a flow cytometry analysis of co-cultures of HEK293 cells (labeled with calcein green) and Raji cells (labeled with calcein red) in the presence of 2M24/anti-hCD70 bispecific or isotype control (left). Coupling of HEK293 and Raji cells is indicated by a double-positive signal (green+red+; square box). Also shown is the coupling efficiency, which is quantified as the percentage of total target cells (Raji) that forms doublets with HEK293 cells (right). Bars represent mean±s.d.; n=2 replicates. FIG. 14B shows the results of similar experiments performed to assess the coupling of human M0 macrophages and Raji cells induced by anti-Dectin-1/anti-hCD20 bispecific. Bars represent mean±s.d.; n=2 replicates. The 2M24/anti-hCD20 bispecific induced coupling of Dectin-1-expressing cells and CDC20-positive B cells (Raji cells).

FIG. 15 shows the results of a secreted alkaline phosphatase reporter assay by Dectin-1 in HEK-Blue hDectin-1a cells using an anti-Dectin-1/anti-CD20 bispecific in the presence of Raji cells. A 2M24 (hIgG4)/a-CD20 bispecific antibody was incubated with Raji cells, after which it was washed twice to remove unbound bispecific antibody. The Raji cells were then mixed with HEK-Blue hDectin-1a cells at a ratio of 200.000 Raji cells to 100.000 HEK cells for 22 hours. Secreted alkaline phosphatase was evaluated at OD 630 nm in the supernatant. Bars represent mean±s.d.; n=2 replicates. Raji cells coated with an anti-Dectin-1/anti CD20 bispecific induced alkaline phosphatase secretion in HEK-Blue hDectin-1a cells.

FIG. 16 shows the induction of Raji cell phagocytosis by Dectin-1-expressing HEK 293 cells by anti-Dectin-1/anti-hCD20 bispecific antibodies. Representative Incucyte images illustrating phagocytosis of Raji cells by HEK cells (arrowhead) at 16 h versus 0 h are shown (left). Co-localization is indicated by yellow fluorescence. Reduction in calcein red signal of Raji cells at 16 h indicates phagocytosis-mediated cell death. Quantification of overlap or co-localization of HEK (calcein green) and Raji (calcein red) in different treatment groups are shown (right). Pre-incubation of HEK cells with ADCP inhibitor Latrunculin A blocks phagocytosis mediated by 15E2/anti-hCD20 bispecific antibody. (n=2 replicates).

FIG. 17 shows coupling of Dectin-1- and HER2-expressing cells induced by anti-Dectin-1/anti-hHER2 bispecific antibodies. Shown are a flow cytometry analysis of co-cultures of Dectin-1-expressing HEK 293 cells (labeled with calcein green) and HER2-expressing SKBR3 cells (labeled with pHrodo red) in the presence of 15E2/anti-hHER2 bispecific or isotype control (left). Coupling of HEK 293 and SKBR3 cells is indicated by a double-positive signal (green+red+; square box). Also shown is the coupling efficiency, which is quantified as the percentage of total target cells (SKBR3) that forms doublets with the Dectin-1 expressing cells (right). Bars represent mean±s.d.; n=2 replicates. Anti-Dectin-1/anti-hHER2 bispecific induces coupling of Dectin-1- and HER2-positive cancer cells.

FIG. 18 shows coupling of Dectin-1-expressing HEK293 cells and CD94-expressing BaF3 cells induced by anti-Dectin-1/anti-hCD94 bispecific induces. Shown are a flow cytometry analysis of co-cultures of HEK293 cells (labeled with calcein green) and BaF3 cells (labeled with pHrodo red) in the presence of 2M24/anti-hCD94 bispecific or isotype control (left). Coupling of HEK293 and BaF3 cells is indicated by a double-positive signal (green+red+; square box). Also shown is the coupling efficiency, which is quantified as the percentage of total target cells (BaF3) that forms doublets with HEK293 cells (right). Bars represent mean±s.d.; n=2 replicates. Anti-Dectin-1/anti-hCD94 bispecific induced coupling of Dectin-1- and CD94-expressing cells.

FIGS. 19A-19B show a schematic illustration of Fab 2M24-mSA or full length 2M24-mSA bound to a biotinylated target antibody. FIG. 19A shows chimeric fusions of monomeric Streptavidin (mSA) and Fab 2M24 or full length 2M24. mSA is genetically fused to either Fab 2M24 or full length 2M24. FIG. 19B shows the coupling of Fab 2M24-mSA or 2M24-mSA to biotinylated target antibodies. The chimeric fusions are incubated with biotinylated target antibodies to generate a bispecific comprising a Dectin-1-binding arm and a second arm binding a target receptor or protein of interest.

FIGS. 20A-20C show the biochemical and functional characterization of Fab 2M24-mSA fusion protein. FIG. 20A shows an HPLC characterization of recombinant Fab 2M24-mSA. FIG. 20B shows an SDS-PAGE analysis of purified Fab 2M24-mSA under reducing conditions. FIG. 20C shows a flow cytometry characterization of Fab 2M24-mSA binding to HEK 293 cells stably overexpressing human Dectin-1 (EC₅₀=1.45 nM). Fab 2M24 fusion to monomeric streptavidin binds to Dectin-1-expressing cells with an affinity of 1.45 nM.

FIGS. 21A-21B shows the phagocytosis of pHrodo-labeled polystyrene biotin beads conjugated with a Fab-2M24 anti-Dectin-1 antibody tagged with monomeric streptavidin (Fab-2M24-mSA). FIG. 21A shows duplet formation of HEK-Blue hDectin-1a cells with Fab-2M24-mSA conjugated to biotin beads and phagocytosis of the beads, assessed by flow cytometry. FIG. 21B shows the phagocytosis of phrodo biotin beads (˜3 μm) conjugated to Fab-2M24-mSA assessed by IncuCyte live imaging (top), as well as representative images of pHrodo positive cells at 3 hours of phagocytosis (engulfed beads fluoresce brightly red in phagosomes) vs. no bead controls (bottom). Fab 2M24-mSA fusion induced binding and phagocytosis of beads by Dectin-1-expressing HEK 293 cells.

FIGS. 22A-22D show bispecific complexes comprising Fab 2M24-mSA and target biotinylated antibodies. Depicted are the HPLC analyses of Fab 2M24-mSA in complex with biotinylated anti-hCD20 (FIG. 22A), biotinylated anti-hCD19 (FIG. 22B), biotinylated anti-hCD70 (FIG. 22C), or biotinylated anti-Amyloid β 1-42 (FIG. 22D). Each panel contains superposition of A280 traces including Fab 2M24-mSA alone, target biotinylated antibody alone, and Fab 2M24-mSA in complex with biotinylated target antibody.

FIG. 23 shows coupling of Dectin-1-expressing HEK293 cells and CD20-expressing Raji cells induced by Fab 2M24-mSA/biotin anti-hCD20 bispecific antibodies. Shown are the flow cytometry analysis of co-cultures of HEK293 (labeled with calcein green) and Raji (labeled with calcein red) in the presence of Fab 2M24-mSA/biotin anti-hCD20 bispecific or isotype bispecific control (left). Co-cultures were incubated at 4° C. or 37° C. Coupling of HEK293 and Raji cells is indicated by a double-positive signal (green+red+; dotted-square). Also shown is the coupling efficiency, which is quantified as the percentage of total target cell (Raji) that forms doublets (right). Bars represent mean±s.d.; n=4 replicates. Fab 2M24-mSA/biotin anti-hCD20 bispecific induced coupling of Dectin-1-expressing HEK293 cells and Raji cells.

FIGS. 24A & 24B show a bispecific antibody design for human bispecific antibodies (e.g., human IgG1 bispecific antibodies) targeting Dectin-1 and a disease target or antigen. FIG. 24A provides a diagram of the design. One arm (2M24A.X) with VH domain A and VL domain B targets human Dectin-1, while the other arm (2M24B.X) with VH domain C and VL domain D targets a disease target or antigen. FIG. 24B provides a diagram of an exemplary mechanism of action for an anti-Dectin-1 bispecific antibody with an active Fc domain, which targets hDectin-1 (via the first arm) on myeloid cells, an antigen on a target cell/disease-causing agent (via the second arm), and Fc receptors on myeloid and NK cells, eliciting robust immune stimulation and phagocytosis.

FIGS. 25A & 25B show that a bispecific antibody with one arm targeting hDectin-1 and the other arm targeting hCD20 (using the variable domains of rituximab) binds to cells expressing human Dectin-1 or human CD20. FIG. 25A (top panel) shows binding of the bispecific antibody targeting hDectin-1 and hCD20 (2M24/CD20), or a bispecific antibody targeting hDectin-1 and RSV (2M24/RSV), to HEK293 cells stably expressing human Dectin-1, as assessed by flow cytometry. FIG. 25A (bottom panel) shows binding of the bispecific antibody 2M24/RSV hIgG1-FITC conjugated and 2M24 bivalent hIgG1-FITC conjugated to PBMCs, as assessed by flow cytometry. FIG. 25B shows binding of rituximab (human IgG1), 2M24/CD20 with active human IgG1 Fc, 2M24/CD20 with inert human IgG1 Fc, 2M24/RSV with active human IgG1 Fc, or 2M24/RSV with inert human IgG1 Fc to CD20-expressing B cell lymphoma Raji cell line.

FIGS. 26A & 26B show that bispecific antibody targeting hDectin-1 and hCD20 (2M24/CD20) induces coupling of Dectin-1- and CD20-expressing cells. FIG. 26A: To assess coupling of Dectin-1-expressing HEK293 cells (effector) and CD20-expressing Raji cells (target), cells were differentially labeled with calcein green (effector) or calcein red (target) dyes. Labeled cells were co-cultured and treated with hIgG1 inert 2M24/CD20 or 2M24/RSV (control) bispecific antibody to induce effector:target coupling. Successful coupling of effector:target cells is indicated by the double-positive staining (Calcein green+, calcein red+, square box). FIG. 26B: Dose-titration of bispecifics in co-cultures of effector:target cells. Coupling efficiency is quantified as the percentage of total target cells that binds or couples to effector cells.

FIGS. 27A & 27B show that bispecific antibody targeting hDectin-1 and hCD20 (2M24/CD20) with an active hIgG1 Fc does not induce monocyte depletion by antibody dependent-cellular cytotoxicity (ADCC) or antibody-dependent cellular phagocytosis (ADCP). PBMCs from two healthy donors—donor 76 (FIG. 27A) and donor 77 (FIG. 27B) were treated with increasing concentrations of 2M24/CD20 bispecific antibody (hIgG1 active or inert isotypes) and rituximab for 24 h, and subsequently analyzed by flow cytometry to quantify the levels of live, CD14+ monocytes remaining (as a % of isotype controls).

FIGS. 28A & 28B show that bispecific antibody targeting hDectin-1 and hCD20 (2M24/CD20) with an active hIgG1 Fc elicits superior B cell depletion compared to Rituximab. PBMCs from two healthy donors—donors 83 (FIG. 28A) and 84 (FIG. 28B)—were treated with increasing concentrations of the indicated antibodies for 24 h, and subsequently analyzed by flow cytometry to quantify the levels of remaining live, CD19+ B cells (reported as a % of B cells in isotype control-treated PBMCs).

FIGS. 29A & 29B show that Rituximab induces higher B cell shaving (CD19 downregulation) compared to 2M24/CD20 active IgG1 bispecific antibody. Expression of CD19+ on B cells from two healthy donors-donor 83 (FIG. 29A) and donor 84 (FIG. 29B)—was quantified by flow cytometry following a 24-hour incubation with increasing concentration of 2M24/CD20 hIgG1 (active isotype) bispecific antibody, Rituximab, or isotype controls. The mean fluorescent intensity (MFI) for CD19 staining using anti-CD19 (BV605 conjugated) was used to evaluate the effect of 2M24/CD20 bispecific and Rituximab on CD19 expression on B cells. EC50 values were calculated based on non-linear regression analysis.

FIG. 30 shows differential cytokine release induced by 2M24/CD20 active IgG1 bispecific antibody as compared to rituximab. ELISA-based (mesoscale discovery) quantification of cytokines was undertaken in supernatants isolated from healthy donor PBMCs treated with 2M24/CD20 active hIgG1 bispecific, Rituximab, or isotype controls. PBMCs were stimulated with antibodies overnight, and supernatants were subsequently analyzed by MSD. Cytokines tested were IFNγ, IL-12p70, IL-6, TNFα, IL-1β, IL-4, IL-13, IL-10, and IL-8. Each plot shows cytokine secretion (in pg/mL) as a function of antibody used for treatment (from left to right: 2M24/CD20 hIgG1 bispecific, 2M24/RSV hIgG1 bispecific, rituximab hIgG1, and isotype control hIgG1).

FIGS. 31A & 31B show that 2M24/CD20 hIgG1 (active isotype) bispecific antibody induces superior B-cell depletion and lower CD19 shaving compared to Rituximab in co-cultures of human macrophages and GFP-expressing Raji B cells. FIG. 31A: Flow cytometry analysis of co-cultures of human macrophages and Raji-GFP cells (3:1 ratio) in the presence of 2M24/CD20 hIgG1 (active isotype) bispecific, 2M24/RSV control, fucosylated Rituximab or isotype hIgG1 control. Co-cultures were incubated at 37° C. for 24 hours and then stained with a PE a-CD206 Ab to label macrophages and a BV-605 a-CD19 antibody to label Raji cells. The number of the remaining live/Raji-GFP+ cells was assessed in the end of the experiment. The primary antibodies were used in a serial dose titration. FIG. 31B: Assessment of CD19 on Raji-GFP cells after 24 hours. B-cell receptor shaving is shown as the reduction in the CD19 MFI in the presence of a-Dectin-1/a-hCD20 bispecific or Rituximab.

FIGS. 32A-32C show that 2M24/CD20 active IgG1 bispecific antibody induces superior tissue B cell depletion as compared to Rituximab in single cell suspension of kidney cancer biopsies. Single cell suspensions from two Kidney cancer tissue biopsies were analyzed by flow cytometry in the presence of 2M24/CD20 hIgG1 (active or inert) bispecific antibody, 2M24/RSV hIgG1 controls, fucosylated Rituximab, and respective isotype controls. Kidney cancer tissue biopsies were dissociated to single cell suspensions and treated with primary antibodies (2 μg/ml) for 24 hours at 37° C. Immune cell populations were analyzed by flow cytometry. Cells were initially gated for live cells, further separated into CD45+ cells (immune cells) and CD45− cells (non-immune cells), and then CD19+ (B cells) and CD3+ (T Cells) cells were identified within the CD45+ population (FIGS. 32A & 32B). The number of the remaining B cells was assessed by an anti-CD19 antibody and expressed as percentage of the CD45+ immune cell population (FIG. 32C).

FIGS. 33A-33C show that Anti-Dectin 1 antibody (clone 2M24) induces Dectin 1-clustering and TNFα secretion from human macrophages. Cytokine secretion by cultured macrophages and single cell suspension of kidney cancer biopsies stimulated with immobilized anti-Dectin-1 antibody (clone 2M24) or 2M24/CD20 bispecific antibody was tested. The anti-Dectin-1 antibody (clone 2M24), isotype control or the 2M24/CD20 bispecific antibody were immobilized overnight in U-bottomed polypropylene microtiter plates at 10 ug per well, followed by culture of human monocyte-derived macrophages (FIGS. 33A & 33B) or single cell suspension from kidney cancer biopsy (FIG. 33C). The cells were cultured for 24 hours and evaluation of TNFα secretion in the supernatant was assessed by ELISA. As a positive control, cells were stimulated with zymosan.

FIG. 34 shows that immobilized anti-Dectin 1 antibody (clone 2M24) promotes immune stimulation in single cell suspension of kidney cancer biopsies. Single-cell suspensions from kidney cancer biopsies were treated with immobilized anti-Dectin-1 antibody (clone 2M24) or isotype control hIgG4 antibody for 24 h. Supernatants were analyzed by ELISA for the release of various cytokines, including IFNγ, IL-6, TNFα, IL-23, IL-12p70, IL-10, and IL-13. Each plot shows amount of cytokine (pg/mL) as a function of antibody treatment. Shown are results from treatment with anti-Dectin-1 antibody (clone 2M24) or isotype control hIgG4 antibody using kidney cancer donor 3 (left) or donor 4 (right).

FIG. 35 shows the effect of 2M24/CD20 bispecific antibody on CD16 expression in human NK cells, as compared to rituximab or isotype control (RSV). Results indicate that CD16 antigen levels on NK cells are better maintained in PBMCs treated with the 2M24/CD20 bispecific compared to rituximab.

FIG. 36 shows the effect of 2M24/CD20 bispecific antibody on CD19 expression in human B cells, as compared to rituximab or isotype control (2M24/RSV bispecific). Results indicate that CD19 antigen levels are better maintained on B cells treated with the 2M24/CD20 bispecific compared to rituximab.

FIG. 37 shows depletion of human B cells by 2M24/CD20 bispecific antibody derived from rituximab or 2M24/CD20 bispecific antibody derived from obinutuzumab. Results indicate that the 2M24/CD20 bispecific derived from the rituximab arm is better at depleting B cells compared to the bispecific derived from obinutuzumab.

FIG. 38 shows the design of an exploratory study on safety and efficacy of 2M24/CD20 bispecific antibody in non-human primates.

FIGS. 39 & 40 show depletion of circulating B cells in cynomolgus monkeys by 2M24/CD20 hIgG1 bispecific antibody generated in cells treated with kifunensine (KIF). FIG. 39: B cell depletion in monkeys treated with 5 mg/kg 2M24/CD20 hIgG1 KIF (upper) or 2M24/CD20 hIgG1 inert (lower). FIG. 40: B cell depletion in monkeys treated with 5 mg/kg rituximab hIgG1 KIF.

FIGS. 41A & 41B show depletion of tissue-resident B cells in cynomolgus monkeys by 2M24/CD20 hIgG1 bispecific antibody generated in cells treated with kifunensine (KIF). FIG. 41A: B cell depletion in bone marrow of monkeys treated with 5 mg/kg 2M24/CD20 hIgG1 KIF or rituximab hIgG1 KIF. FIG. 41B: B cell depletion in lymph nodes of monkeys treated with 5 mg/kg 2M24/CD20 hIgG1 KIF or rituximab hIgG1 KIF.

FIG. 42 shows depletion of B cells from cynomolgus monkey PBMCs ex vivo.

FIG. 43 shows the format of a bispecific molecule that uses knobs-into-holes technology to pair an anti-CD20 conventional half-antibody with an anti-Dectin-1 single chain variable fragment (scFv) Fc fusion arm (2M24 scFv/CD20). H: 2M24 VH domain; L: 2M24 VL domain.

FIGS. 44A-44C show purification and functional characterization of the 2M24/CD20 bispecific antibody. FIG. 44A shows purification of the molecule by size exclusion chromatography (SEC). FIG. 44B shows that purified bispecific antibody promoted targeted immune stimulation, as assessed in an NFκB reporter assay. FIG. 44C shows human B cell depletion by the 2M24 scFv/CD20 bispecific antibody.

FIG. 45 shows depletion of B cells from healthy donor PBMCs by CD20 targeting antibodies, including rituximab, a CD3×CD20 bispecific T cell engager, and a bispecific binding molecule comprising an anti-CD20 conventional half-antibody arm with an anti-Dectin-1 single chain variable fragment (scFv) arm (2M24 scFv/CD20) in a non-fucosylated hIgG1 Fc format. PBMCs isolated from a health donor were incubated with CD20-targeting antibodies for 24 h and subsequently stained with an anti-CD19 (B-cell specific marker) antibody to characterize remaining B cells by flow cytometry. Data are presented as a percentage of remaining B cells relative to the control untreated group. NF: non-fucosylated.

FIG. 46 shows depletion of B cells from a prostate cancer tumor biopsy specimen by rituximab, a bispecific binding molecule comprising an anti-CD20 conventional half-antibody arm with an anti-Dectin-1 single chain variable fragment (scFv) arm (2M24 scFv/CD20) in a non-fucosylated hIgG1 Fc format, 2M24×CD20 hIgG1 bispecific (DuetMab format), or isotype control. Single-cell suspension generated from a prostate cancer biopsy was treated with CD20− targeting antibodies for 24 hours. Cells were subsequently stained with antibodies against CD45, CD3, CD11b, CD16, CD163, and CD19. B cells were identified as CD45+CD3−CD19+. Data show the percentage of remaining B cells relative to the number of B cells in the isotype control group (RSV hIgG1).

FIG. 47A shows the effect of 2M24×CD20 bispecific binding protein (DuetMab format) treatment on CD16 expression on NK cells from healthy human PBMCs, as compared to rituximab or control antibody. CD16 levels on NK cells were better maintained by 2M24×CD20 bispecific treatment than rituximab.

FIG. 47B shows the effect of 2M24×CD20 bispecific binding protein treatment on CD19 expression on B cells from healthy human PBMCs, as compared to rituximab or control antibody. Results from 3 donors are shown; for each donor, the order of data points is: 2M24×CD20 bispecific, rituximab, control (left to right). CD19 levels on B cells were better maintained by 2M24×CD20 bispecific treatment than rituximab.

FIG. 47C shows the effect of 2M24×CD20 bispecific binding protein treatment on B cell depletion from healthy human PBMCs, as compared to rituximab, obinutuzumab or control antibody. Two forms of 2M24×CD20 bispecific binding protein were tested: one with variable domains from rituximab, and one with variable domains from Obinutuzumab. B cells were quantified relative to an untreated control (dotted line). 2M24×CD20 bispecific binding protein with anti-CD20 arm from rituximab showed better B cell depletion than a comparable 2M24 bispecific with anti-CD20 arm from Obinutuzumab.

FIG. 47D shows the effect of 2M24×CD20 bispecific binding protein treatment on B cell depletion using a single-cell suspension from a kidney cancer biopsy, as compared to 2M24/RSV bispecific, rituximab, isotype control antibody, or untreated. 2M24×CD20 bispecific binding protein induced superior B cell depletion as compared to rituximab.

FIG. 48 shows the design for an exploratory study on 2M24×CD20 bispecific binding protein in non-human primates (cynomolgus monkey). Timepoints for whole blood collection, tissue collection, whole blood collection for chemistry and coagulation, and body weight measurement are indicated. CBC: complete blood count. PK: pharmacokinetics. BM: bone marrow. LN: lymph node.

FIGS. 49A-49C show depletion of B cells by 2M24×CD20 bispecific binding protein in the non-human primate study. FIG. 49A shows depletion of CD19+ B cells in blood by 2M24×CD20 hIgG1 bispecific binding protein treated with KIF (upper left), 2M24×CD20 bispecific binding protein with inert hIgG1 Fc (lower left), or rituximab hIgG1 treated with KIF (upper right). FIG. 49B shows depletion of B cells (% of CD45+) in bone marrow (top) or lymph nodes (bottom) by 2M24×CD20 hIgG1 bispecific binding protein treated with KIF, 2M24×CD20 bispecific binding protein with inert hIgG1 Fc, or rituximab hIgG1 treated with KIF, as indicated. FIG. 49C shows depletion of B cells ex vivo from cynomolgus PBMCs treated with serial dilutions of 2M24×CD20 hIgG1 bispecific binding protein treated with KIF, rituximab hIgG1 treated with KIF, or control antibody treated with KIF.

FIGS. 50A & 50B show the results of a mouse study on anti-mouse Dectin-1 antibody 2A11×mCD20 bispecific binding protein for depletion of B cells. FIG. 50A shows the design for the study. FIG. 50B shows the results of treatment with 2A11×mCD20 bispecific mIgG1 binding protein, anti-mouse CD20 rat IgG2a antibody, or mIgG1 isotype control on CD19+ B cells (as % of total CD45+ cells) from blood, peritoneum, bone marrow, and spleen. Differences between study groups were analyzed by ordinary one-way Anova with Tukey's test. * P≤0.05; ** P≤0.01; *** P≤0.001; **** P≤0.0001.

FIGS. 51A-51E show the results of ex vivo testing of 2M24×CD20 bispecific binding protein for various properties. FIG. 51A shows the activation of Dectin-1-expressing reporter HEK cell line as a function of concentration of antibody. Shown are results from treatment with 2M24×CD20 bispecific binding protein (circles), rituximab (squares), or D-zymosan (triangles), an established ligand for Dectin-1 used as a positive control. FIG. 51B shows the induction of phagocytosis of B cell lines (% B cells relative to isotype control treatment) as a function of concentration of antibody. Shown are results from treatment with 2M24×CD20 bispecific binding protein (black circles), rituximab (triangles), or non-specific RSV antibody (grey circles). FIG. 51C shows B cell depletion (% B cells relative to untreated control) as a function of concentration of antibody. Shown are results from treatment with 2M24×CD20 bispecific binding protein (black squares), rituximab (triangles), an anti-CD20/anti-CD3 bispecific engager (gray squares), or non-specific RSV antibody (grey circles). FIGS. 51D & 51E show ex vivo cytokine stimulation (pg/mL of the indicated cytokines) from human PBMCs by 2M24×CD20 bispecific binding protein or comparator anti-CD20 antibodies. Shown are results from treatment with 2M24×CD20 bispecific binding protein, rituximab, an anti-CD20/anti-CD3 bispecific engager, non-specific RSV antibody, or untreated control. Cytokines measured included IL-6 (FIG. 51D, upper left), TNF-α (FIG. 51D, lower left), IFN-γ (FIG. 51D, right), IL-12p70 (FIG. 51E, upper left), IL-1β (FIG. 51E, lower left), and IL-2 (FIG. 51E, right).

FIGS. 52A-52H show the results of in vivo testing of surrogate anti-mDectin-1/anti-mCD20 bispecific antibody in various mouse models. FIG. 52A shows the effect of surrogate anti-mDectin-1/anti-mCD20 mIgG2a bispecific antibody (squares) or isotype control (circles) treatment on tumor volume (mm³) over time (days post-injection) in in vivo mouse xenograft models using Ramos B cell lymphoma xenografts in SCID mice (upper left), Daudi B cell lymphoma xenografts in SCID mice (lower left), or Raji B cell lymphoma xenografts in SCID-beige mice. Efficacy data are show as mean+/−SEM. FIG. 52B shows myeloid cell activation (indicated myeloid cells as % of CD45+ cells) of the indicated cell populations in spleen (left), lymph nodes (middle), or tumor (right) in mice implanted with MC38 murine colon carcinoma cells expressing human CD20. For each cell population, cell numbers as % of total CD45+ cells are provided, with data from isotype control on left and anti-mDectin-1/anti-mCD20 on right. GRNs: granulocytes. DCs: dendritic cells. Macs: macrophages. Differences between study groups were analyzed by two-way Anova. * P≤0.05; ** P≤0.01; *** P≤0.001; **** P≤0.0001. FIG. 52C shows activation of CD3+ T cells, Ki67+ T cells, effector T cells, and splenic T cells expressing the indicated markers in the MC38 xenograft mouse model upon treatment with anti-mDectin-1/anti-mCD20 mIgG1 bispecific (right in all panels) or isotype control (left in all panels). For each cell population, cell numbers as % of indicated cell population are provided. Differences between study groups were analyzed by two-way Anova. * P≤0.05; ** P≤0.01; *** P≤0.001; **** P≤0.0001. FIG. 52D shows activation of intratumoral CD3+ T cells (as % of tumor CD45+ cells; left), intratumoral CD4+ or CD8+ T cells (as % of tumor T cells; middle), and intratumoral CD8+ T cell production of cytokines IL-2 and GmzB in the MC38 xenograft mouse model upon treatment with anti-mDectin-1/anti-mCD20 mIgG1 bispecific (right in all panels) or isotype control (left in all panels). Differences between study groups were analyzed by two-way Anova. * P≤0.05; ** P≤0.01; *** P≤0.001; **** P≤0.0001. FIG. 52E shows B cell depletion in blood (upper left), lymph node (upper right), spleen (lower left), and bone marrow (lower right) upon treatment with anti-mDectin-1/anti-mCD20 mIgG2a bispecific (gray circles) or isotype control (black circles) in naïve C57BL/6 mice. Data are depicted as B cells (% of B cells in isotype control) on the indicated day post-injection. FIG. 52F shows B cell depletion in lung (upper left), liver (upper right), brain (lower left), and heart (lower right) upon treatment with anti-mDectin-1/anti-mCD20 mIgG2a bispecific (gray circles) or isotype control (black circles) in naïve C57BL/6 mice. Data are depicted as B cells (% of B cells in isotype control) on the indicated day post-injection. FIG. 52G compares tumor growth inhibition of anti-mDectin-1/anti-mCD20 bispecific with active Fc (mIgG2a) and inert mIgG2a Fc vs. isotype control in a Daudi xenograft model in SCID mice. Tumor volume (mm³) was plotted over time (days post-implant). Data from isotype control (circles), anti-mDectin-1/anti-mCD20 mIgG2a active (squares), and anti-mDectin-1/anti-mCD20 mIgG2a inert (triangles) as shown. Error bars indicate SEM. FIG. 52H shows proportion of resident monocytes (CD11b^{hi}MHCII-Ly6c−; upper left), inflammatory monocytes (CD11b^{hi}MHCII-Ly6c+; upper right), macrophages (CD11b+F4/80+; lower left), and M1 and M2 macrophages (lower right) in tumor tissue (as % of CD45+ for all plots, except for lower right, which shows % of macrophages) upon treatment with mIgG2a isotype control (circles) or anti-mDectin-1/anti-mCD20 bispecific with mIgG2a inert Fc (triangles).

FIGS. 53A-53I show the results of in vivo testing of 2M24×CD20 bispecific binding protein in a cynomolgus monkey model. FIG. 53A shows level of B cells in peripheral blood (% relative to baseline) over time after administration of 2M24×CD20 bispecific at 1 mg/kg (upper left), 10 mg/kg (lower left), or 100 mg/kg (right). FIG. 53B shows level of B cells in lymphoid organs bone marrow (upper) and lymph node (lower) over time after administration of 2M24×CD20 bispecific at 1 mg/kg (left), 10 mg/kg (middle), or 100 mg/kg (right). FIG. 53C shows level of B cells (as % of tissue CD45+ cells) in indicated lymphoid (left) and non-lymphoid (right) tissues at day 15 after treatment with vehicle control (left for all tissues) or 100 mg/kg 2M24×CD20 bispecific (right for all tissues). FIGS. 53D & 53E show depletion of the indicated B cell subsets in spleens at day 15 after treatment with vehicle control (lower in FIG. 53D; left in FIG. 53E) or 100 mg/kg 2M24×CD20 bispecific (upper in FIG. 53D; right in FIG. 53E). FIG. 53F shows levels of classical dendritic cells in circulation (as % of CD45+ cells) over time (days) after treatment with 2M24×CD20 bispecific (black circles) or rituximab-like anti-CD20 mAb (gray circles). FIG. 53G shows levels of classical dendritic cells in circulation (as % of CD45+ cells) over time (days) after treatment with 2M24×CD20 bispecific at 1 mg/kg (black circles) or 10 mg/kg (gray squares). FIGS. 53H & 53I show levels of pro-inflammatory cytokines in serum of cynomolgus monkeys over time after administration of three weekly doses of 2M24×CD20 bispecific binding protein at 1 mg/kg (FIG. 53H), 10 mg/kg (FIG. 53H), or 100 mg/kg (FIG. 53I).

### DETAILED DESCRIPTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing”, and is inclusive or open-ended.

Any reference to “or” herein is intended to encompass “and/or” unless otherwise stated. As used herein, the term “about” with reference to a number refers to that number plus or minus 10% of that number. The term “about” with reference to a range refers to that range minus 10% of its lowest value and plus 10% of its greatest value.

### I. Multispecific Binding Proteins

In certain aspects, the present disclosure provides antigen binding domains that bind to human Dectin-1, as well as multispecific (e.g., bispecific) binding molecules comprising the same. In certain aspects, the present disclosure provides multispecific (e.g., bispecific) antibodies and antibody fragments comprising a first antigen-binding domain that binds to a first target of interest (i.e., Dectin-1) and a second antigen-binding domain that binds to a second target of interest (i.e., CD20). In some embodiments, the present disclosure provides multispecific (e.g., bispecific) antibodies and antibody fragments comprising a first antigen-binding domain that binds to human Dectin-1 and a second antigen-binding domain that binds to CD20.

In some embodiments, the multispecific binding protein comprises a first polypeptide chain comprising the amino acid sequence QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLWCLVKGFYPSDIAVEWESN GQPENNYKTTPPVLDSDGSFFLYSKLTVDKSRWQQGNVFSCSVMHEALHNHYTQKSLS LSPG (SEQ ID NO:31) or QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLWCLVKGFYPSDIAVEWESN GQPENNYKTTPPVLDSDGSFFLYSKLTVDKSRWQQGNVFSCSVMHEALHNHYTQKSLS LSPGK (SEQ ID NO:35); a second polypeptide chain comprising the amino acid sequence QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLSCAVKGFYPSDIAVEWESNGQPENNYKTTPPVLDSDGSFFLVS KLTVDKSRWQQGNVFSCSVMHEALHNHYTQKSLSLSPG (SEQ ID NO:32) or QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLSCAVKGFYPSDIAVEWESNGQPENNYKTTPPVLDSDGSFFLVS KLTVDKSRWQQGNVFSCSVMHEALHNHYTQKSLSLSPGK (SEQ ID NO:36); and a third polypeptide chain comprising the amino acid sequence QIVLSQSPAILSASPGEKVTMTCRASSSVSYIHWFQQKPGSSPKPWIYATSNLASGVPVRF SGSGSGTSYSLTISRVEAEDAATYYCQQWTSNPPTFGGGTKLEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO:33).

In some embodiments, the multispecific binding protein comprises a first polypeptide chain comprising the amino acid sequence QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLSCAVKGFYPSDIAVEWESN GQPENNYKTTPPVLDSDGSFFLVSKLTVDKSRWQQGNVFSCSVMHEALHNHYTQKSLS LSPG (SEQ ID NO:37) or QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLSCAVKGFYPSDIAVEWESN GQPENNYKTTPPVLDSDGSFFLVSKLTVDKSRWQQGNVFSCSVMHEALHNHYTQKSLS LSPGK (SEQ ID NO:39); a second polypeptide chain comprising the amino acid sequence QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLWCLVKGFYPSDIAVEWESNGQPENNYKTTPPVLDSDGSFFLY SKLTVDKSRWQQGNVFSCSVMHEALHNHYTQKSLSLSPG (SEQ ID NO:38) or QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLWCLVKGFYPSDIAVEWESNGQPENNYKTTPPVLDSDGSFFLY SKLTVDKSRWQQGNVFSCSVMHEALHNHYTQKSLSLSPGK (SEQ ID NO:40); and a third polypeptide chain comprising the amino acid sequence QIVLSQSPAILSASPGEKVTMTCRASSSVSYIHWFQQKPGSSPKPWIYATSNLASGVPVRF SGSGSGTSYSLTISRVEAEDAATYYCQQWTSNPPTFGGGTKLEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO:33).

As is known in the art, the C-terminal lysine of some antibody heavy chain species may be cleaved off in some fraction of molecules. In some embodiments, one or both of the antibody Fc domains do not have a C-terminal lysine.

In some embodiments, the first polypeptide chain comprises the amino acid sequence of SEQ ID NO:31, the second polypeptide chain comprises the amino acid sequence of SEQ ID NO: 32, and the third polypeptide chain comprises the amino acid sequence of SEQ ID NO:33. In some embodiments, the first polypeptide chain comprises the amino acid sequence of SEQ ID NO: 35, the second polypeptide chain comprises the amino acid sequence of SEQ ID NO:36, and the third polypeptide chain comprises the amino acid sequence of SEQ ID NO:33. In some embodiments, provided herein is a composition comprising a mixture of multispecific binding protein species, wherein each species comprises a first polypeptide chain that comprises the amino acid sequence of SEQ ID NO:31 or SEQ ID NO:35, a second polypeptide chain that comprises the amino acid sequence of SEQ ID NO:32 or SEQ ID NO:36, and a third polypeptide chain that comprises the amino acid sequence of SEQ ID NO:33.

In some embodiments, the first polypeptide chain comprises the amino acid sequence of SEQ ID NO:37, the second polypeptide chain comprises the amino acid sequence of SEQ ID NO: 38, and the third polypeptide chain comprises the amino acid sequence of SEQ ID NO:33. In some embodiments, the first polypeptide chain comprises the amino acid sequence of SEQ ID NO: 39, the second polypeptide chain comprises the amino acid sequence of SEQ ID NO:40, and the third polypeptide chain comprises the amino acid sequence of SEQ ID NO:33. In some embodiments, provided herein is a composition comprising a mixture of multispecific binding protein species, wherein each species comprises a first polypeptide chain that comprises the amino acid sequence of SEQ ID NO:37 or SEQ ID NO:39, a second polypeptide chain that comprises the amino acid sequence of SEQ ID NO:38 or SEQ ID NO:40, and a third polypeptide chain that comprises the amino acid sequence of SEQ ID NO:33.

In some embodiments, the first, second, and third polypeptide chains are associated in a multispecific binding protein comprising a first antigen binding domain that binds to human Dectin-1 and a second antigen binding domain that binds to human CD20.

In some embodiments, the multispecific binding protein, antigen binding domain, antibody, or antibody fragment binds to human Dectin-1. In some embodiments, the multispecific binding protein, antigen binding domain, antibody, or antibody fragment binds to human Dectin-1 expressed on the surface of a macrophage, monocyte, dendritic cell, or granulocyte. In some embodiments, the multispecific binding protein, antigen binding domain, antibody, or antibody fragment binds to human Dectin-1 isoform A and/or human Dectin-1 isoform B. In some embodiments, human Dectin-1 isoform A comprises the amino acid sequence MEYHPDLENLDEDGYTQLHFDSQSNTRIAVVSEKGSCAASPPWRLIAVILGILCLVILVIA VVLGTMAIWRSNSGSNTLENGYFLSRNKENHSQPTQSSLEDSVTPTKAVKTTGVLSSPCP PNWIIYEKSCYLFSMSLNSWDGSKRQCWQLGSNLLKIDSSNELGFIVKQVSSQPDNSFWI GLSRPQTEVPWLWEDGSTFSSNLFQIRTTATQENPSPNCVWIHVSVIYDQLCSVPSYSICE KKFSM (SEQ ID NO:9). In some embodiments, human Dectin-1 isoform B comprises the amino acid sequence MEYHPDLENLDEDGYTQLHFDSQSNTRIAVVSEKGSCAASPPWRLIAVILGILCLVILVIA VVLGTMGVLSSPCPPNWIIYEKSCYLFSMSLNSWDGSKRQCWQLGSNLLKIDSSNELGFI VKQVSSQPDNSFWIGLSRPQTEVPWLWEDGSTFSSNLFQIRTTATQENPSPNCVWIHVSV IYDQLCSVPSYSICEKKFSM (SEQ ID NO:10). In some embodiments, the multispecific binding protein, antigen binding domain, antibody, or antibody fragment binds to human Dectin-1 expressed on the surface of a cell with an EC50 of less than 5 nM, less than 2 nM, less than 1 nM, or less than 0.5 nM. In some embodiments, the antigen binding domain, antibody, or antibody fragment is capable of binding to human Dectin-1 and monkey Dectin-1, e.g., cynomolgus Dectin-1.

In some embodiments, the multispecific binding protein, antigen binding domain, antibody, or antibody fragment binds to human CD20, also known as MS4A1, B1, S7, Bp35, FMC7, CVID5, and LEU-16. In some embodiments, human CD20 refers to a polypeptide encoded by NCBI Gene ID No. 931. An exemplary and non-limiting human CD20 polypeptide is provided by NCBI Ref. Seq. NP_068769:

(SEQ ID NO: 34)
MTTPRNSVNGTFPAEPMKGPIAMQSGPKPLFRRMSSLVGPTQSFFMRESK
TLGAVQIMNGLFHIALGGLLMIPAGIYAPICVTVWYPLWGGIMYIISGSL
LAATEKNSRKCLVKGKMIMNSLSLFAAISGMILSIMDILNIKISHFLKME
SLNFIRAHTPYINIYNCEPANPSEKNSPSTQYCYSIQSLFLGILSVMLIF
AFFQELVIAGIVENEWKRTCSRPKSNIVLLSAEEKKEQTIEIKEEVVGLT
ETSSQPKNEEDIEIIPIQEEEEEETETNFPEPPQDQESSPIENDSSP.

In some embodiments, antibody and immunoglobulin are used interchangeably and herein are used in the broadest sense and encompass various antibody structures, including but not limited to monoclonal antibodies (e.g., full length or intact monoclonal antibodies), polyclonal antibodies, multispecific antibodies (e.g., bispecific antibodies), antibody fragments and single domain antibody (as described in greater detail herein), so long as they exhibit the desired antigen binding activity.

In some embodiments, antibodies (immunoglobulins) refer to a protein having a structure substantially similar to a native antibody structure, or a protein having heavy and light chain variable regions having structures substantially similar to native heavy and light chain variable region structures. Native antibodies refer to naturally occurring immunoglobulin molecules with varying structures. For example, native immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The subunit structures and three-dimensional configurations of the different classes of immunoglobulins are well known and described generally, for example, in Abbas et al., 2000, Cellular and Mol, and Kindt et al., Kuby Immunology, 6th ed., W.H. Freeman and Co., page 91 (2007). Antibodies (immunoglobulins) are assigned to different classes, depending on the amino acid sequences of the heavy chain constant domains. There are five major classes of antibodies: α (IgA), δ (IgD), ϵ (IgE), γ (IgG), or μ (IgM), some of which may be further divided into subtypes, e.g., γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

In some embodiments, an Fc, Fc region, or Fc domain refers to the C-terminal region of an antibody heavy chain that contains at least a portion of the constant region. The term includes native sequence Fc regions and variant Fc regions. An Fc can refer to the last two constant region immunoglobulin domains (e.g., CH2 and CH3) of IgA, IgD, and IgG, the last three constant region immunoglobulin domains of IgE and IgM, and optionally, all or a portion of the flexible hinge N-terminal to these domains. For IgA and IgM, Fc may include the J chain. An IgG Fc region comprises an IgG CH2 and an IgG CH3 domain and in some cases, inclusive of the hinge. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. Human IgG Fc domains are of particular use in the present disclosure, and can be the Fc domain from human IgG1, IgG2 or IgG4.

Examples of antibody fragments include, but are not limited to, Fab, Fab′, F(ab′)2, and Fv fragments, Fab′-SH, F(ab′)2, diabodies, linear antibodies, single chain antibodies, nanobodies, scFv fragments, VH, and multispecific (e.g., bispecific) antibodies/fragments formed from antibody fragments.

A “Fab” (fragment antigen binding) is a portion of an antibody that binds to antigens and includes the variable region and CH1 of the heavy chain linked to the light chain via an inter-chain disulfide bond.

In some embodiments, the multispecific binding protein or antibody of the present disclosure comprises an Fc region. An antibody/multispecific binding protein may be of any class or subclass, including IgG and subclasses thereof (IgG1, IgG2, IgG3, IgG4), IgM, IgE, IgA, and IgD. An immunoglobulin Fc region of the molecule that causes targeted phagocytosis may have important role in the process by engaging Fc receptors and inducing additional phagocytosis. In some embodiments, the molecule has a modified Fc region that has reduced ADCC activity as compared to a wild type human IgG1 (e.g., comprising one or more mutations reducing effector function as described herein).

In some embodiments, the multispecific binding protein or antibody of the present disclosure comprises an Fc region wherein a carbohydrate structure attached to the Fc region has reduced fucose or lacks fucose, e.g., at least one or two of the heavy chains of the antibody is non-fucosylated. In some embodiments, provided herein is a composition comprising the multispecific binding protein or antibody of the present disclosure that comprises an Fc region wherein a carbohydrate structure attached to the Fc region has reduced fucose or lacks fucose, e.g., at least one or two of the heavy chains of the antibody is non-fucosylated. In some embodiments, less than 50% of the N-glycoside-linked carbohydrate chains in the composition contain a fucose residue. In some embodiments, substantially none of the N-glycoside-linked carbohydrate chains contain a fucose residue. In some embodiments, the multispecific binding protein or antibody with reduced fucose or lacking fucose has improved ADCC function.

In other embodiments, the multispecific binding protein of the present disclosure (e.g., an IgG1 antibody) or composition comprising the multispecific binding protein of the present disclosure (e.g., an IgG1 antibody) comprises wild-type glycosylation of the Fc region. In some embodiments, provided herein are fucosylated binding proteins of the present disclosure (e.g., an IgG1 antibody) or compositions comprising a fucosylated binding protein of the present disclosure (e.g., an IgG1 antibody).

Fucosylation or fucosylated binding proteins can refer to the presence of fucose residues within the oligosaccharides attached to the peptide backbone of an antibody. Specifically, a fucosylated antibody comprises a (1,6)-linked fucose at the innermost N-acetylglucosamine (GlcNAc) residue in one or both of the N-linked oligosaccharides attached to the antibody Fc region, e.g., at position Asn 297 of the human IgG1 Fc region (EU numbering of Fc region residues). Asn297 may also be located about +3 amino acids upstream or downstream of position 297, i.e., between positions 294 and 300, due to minor sequence variations in immunoglobulins. Non-fucosylated or fucose-deficient antibodies have reduced fucose relative to the amount of fucose on the same antibody produced in a cell line. Antibody fucosylation can be measured, e.g., in an N-glycosidase F treated antibody composition assessed by matrix-assisted laser desorption-ionization time-of-flight mass spectrometry (MALDI TOF MS).

In some embodiments, the Fc region comprises one or more mutations that reduce or eliminate fucosylation, e.g., a substitution at Asn 297 of the human IgG1 Fe region (EU numbering of Fc region residues). Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87:614 (2004).

In some embodiments, the afucosylated or non-fucosylated binding protein is produced in a cell line with a genetic modification that results in an afucosylated or non-fucosylated antibody. Examples of cell lines producing afucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87:614 (2004)), cells overexpressing β1,4-N-acetylglucosaminyltransferase III (GnT-III) and Golgi μ-mannosidase II (ManII), and cells with a knockout in the mannosyl-glycoprotein 2-beta-N-acetylglucosaminyltranferase (MGAT1; see Byrne, G. et al. (2018) PLoS Biol. 16: e2005817).

In some embodiments, the afucosylated or non-fucosylated binding protein is produced in a cell line treated with an inhibitor of glycoprocessing enzyme(s), such as kifunensine, which is an inhibitor of mannosidase I (see, e.g., Elbein, A. D. et al. (1990) J. Biol. Chem. 265:15599-15605). For example, cells can be centrifuged and resuspended in growth medium comprising kifunensine (e.g., at 250 μg/mL), then cultured and used for antibody production.

In some embodiments, one or both of the first and second antigen binding domain, antibody, or fragment comprise(s) a tag, e.g., for affinity purification. In some embodiments, the tag is a polyhistidine tag.

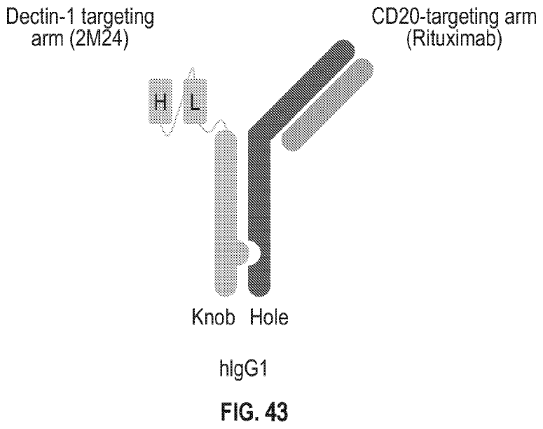

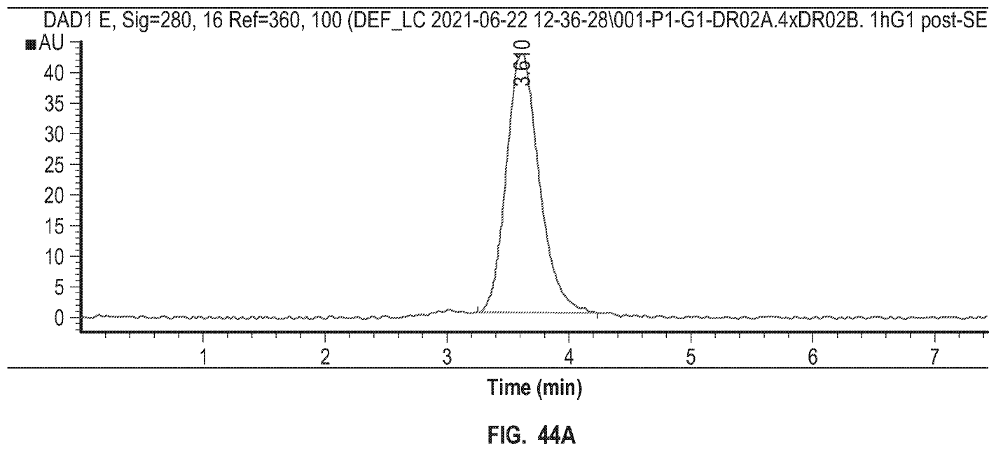

In some embodiments, the multispecific (e.g., bispecific) binding molecule comprises a first antibody arm comprising a single chain variable fragment (scFv) comprising VH and VL domains of the present disclosure that bind to human Dectin-1 and a first Fc region, and a second antibody arm comprising an antibody heavy chain that comprises a VH domain in association with an antibody light chain that comprises a VL domain, and a second Fc region connected to the VH domain. In some embodiments, the scFv arm binds to Dectin-1, and the conventional antibody arm with VH and VL domains on separate polypeptides binds to a target of interest, e.g., as described herein, such as a disease-causing agent. In some embodiments, the first Fc region comprises one or more knob-forming mutations, and the second Fc region comprises one or more cognate hole-forming mutations, or wherein the second Fc region comprises one or more knob-forming mutations, and the first Fc region comprises one or more cognate hole-forming mutations. A non-limiting example of this format is shown in FIG. 43.

In some embodiments, the disease-causing agent is a B cell, tumor or cancer cell, e.g., a malignant B cell. In some embodiments, CD20 is expressed on the surface of a B cell, such as a malignant B cell. CD20 is expressed on most B cells starting from the late pre-B lymphocyte stage. As such, therapies that deplete B cells have targeted CD20 for a variety of indications, such as cancer (e.g., non-Hodgkin's lymphoma or chronic lymphocytic leukemia) and autoimmune conditions (e.g., rheumatoid arthritis, systemic lupus erythematosus (SLE), multiple sclerosis, and Wegener's granulomatosis).

In some embodiments, provided herein is a multispecific (e.g., bispecific) binding molecule that comprises a first antibody arm comprising a single chain variable fragment (scFv) comprising VH and VL domains of the present disclosure that bind to human Dectin-1 and a first Fc region, and a second antibody arm comprising an antibody heavy chain that comprises a VH domain in association with an antibody light chain that comprises a VL domain and a second Fc region connected to the VH domain, wherein the VH and VL domains of the second antibody arm form an antigen binding domain that binds to a target of interest (e.g., a disease causing agent of the present disclosure). In some embodiments, the first Fc region comprises one or more knob-forming mutations, and the second Fc region comprises one or more cognate hole-forming mutations, or the second Fc region comprises one or more knob-forming mutations, and the first Fc region comprises one or more cognate hole-forming mutations. In some embodiments, the scFv comprises a first linker of the present disclosure between the VH and VL domains and a second linker of the present disclosure between the VL domain and the first Fc region. In some embodiments, the first antibody arm comprises the amino acid sequence of

(SEQ ID NO: 31)
QVQLVQSGAEVKKPGASVKVSCKSSGYTFTDYYIHWVRQAPGQGLEWMGW
INPNSGDTNYAQKFQGRITMTRDTSISTAYLELSRLRSDDTAVFYCARNS
GSYSFGYWGQGTLVTVSSGGGGSGGGGSGGGGSGGGGSDIQMTQSPSSVS
ASVGDRVTITCRASQGISSWLAWYQQKPGKAPKLLIFGASSLQSGVPSRF
SGSGSGTDFTLTVSSLQPEDFATYYCQQAYSFPFTFGPGTKVDIEEPKRS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLWCLVK
GFYPSDIAVEWESNGQPENNYKTTPPVLDSDGSFFLYSKLTVDKSRWQQG
NVFSCSVMHEALHNHYTQKSLSLSPG
or
(SEQ ID NO: 37)
QVQLVQSGAEVKKPGASVKVSCKSSGYTFTDYYIHWVRQAPGQGLEWMGW
INPNSGDTNYAQKFQGRITMTRDTSISTAYLELSRLRSDDTAVFYCARNS
GSYSFGYWGQGTLVTVSSGGGGSGGGGSGGGGSGGGGSDIQMTQSPSSVS
ASVGDRVTITCRASQGISSWLAWYQQKPGKAPKLLIFGASSLQSGVPSRF
SGSGSGTDFTLTVSSLQPEDFATYYCQQAYSFPFTFGPGTKVDIEEPKRS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLSCAVK
GFYPSDIAVEWESNGQPENNYKTTPPVLDSDGSFFLVSKLTVDKSRWQQG
NVFSCSVMHEALHNHYTQKSLSLSPG.

In some embodiments, provided herein is a multispecific (e.g., bispecific) binding molecule that comprises a first antibody arm comprising a single chain variable fragment (scFv) comprising VH and VL domains of the present disclosure that bind to human Dectin-1 and a first Fc region, and a second antibody arm comprising an antibody heavy chain that comprises a VH domain in association with an antibody light chain that comprises a VL domain and a second Fc region connected to the VH domain, wherein the VH and VL domains of the second antibody arm form an antigen binding domain that binds to CD20 (e.g., human CD20). In some embodiments, the first Fc region comprises one or more knob-forming mutations, and the second Fc region comprises one or more cognate hole-forming mutations, or the second Fc region comprises one or more knob-forming mutations, and the first Fc region comprises one or more cognate hole-forming mutations. In some embodiments, the scFv comprises a first linker of the present disclosure between the VH and VL domains and a second linker of the present disclosure between the VL domain and the first Fc region. In some embodiments, the first antibody arm comprises the amino acid sequence of QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLWCLVKGFYPSDIAVEWESN GQPENNYKTTPPVLDSDGSFFLYSKLTVDKSRWQQGNVFSCSVMHEALHNHYTQKSLS LSPG (SEQ ID NO:31). In some embodiments, the second antibody arm comprises a second polypeptide comprising the sequence of QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLSCAVKGFYPSDIAVEWESNGQPENNYKTTPPVLDSDGSFFLVS KLTVDKSRWQQGNVFSCSVMHEALHNHYTQKSLSLSPG (SEQ ID NO:32) and a third polypeptide comprising the amino acid sequence of QIVLSQSPAILSASPGEKVTMTCRASSSVSYIHWFQQKPGSSPKPWIYATSNLASGVPVRF SGSGSGTSYSLTISRVEAEDAATYYCQQWTSNPPTFGGGTKLEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO:33). In some embodiments, the first antibody arm comprises the amino acid sequence of QVQLVQSGAEVKKPGASVKVSCKSSGYTFTDYYIHWVRQAPGQGLEWMGWINPNSGD TNYAQKFQGRITMTRDTSISTAYLELSRLRSDDTAVFYCARNSGSYSFGYWGQGTLVTV SSGGGGSGGGGSGGGGSGGGGSDIQMTQSPSSVSASVGDRVTITCRASQGISSWLAWYQ QKPGKAPKLLIFGASSLQSGVPSRFSGSGSGTDFTLTVSSLQPEDFATYYCQQAYSFPFTF GPGTKVDIEEPKRSDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGKEYKCKVS NKALPAPIEKTISKAKGQPREPQVYTLPPSREEMTKNQVSLSCAVKGFYPSDIAVEWESN GQPENNYKTTPPVLDSDGSFFLVSKLTVDKSRWQQGNVFSCSVMHEALHNHYTQKSLS LSPG (SEQ ID NO:37). In some embodiments, the second antibody arm comprises a second polypeptide comprising the sequence of QVQLQQPGAELVKPGASVKMSCKASGYTFTSYNMHWVKQTPGRGLEWIGAIYPGNGD TSYNQKFKGKATLTADKSSSTAYMQLSSLTSEDSAVYYCARSTYYGGDWYFNVWGAG TTVTVSAASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKT KPREEQYNSTYRVVSVLTVLHQDWLNGKEYKCKVSNKALPAPIEKTISKAKGQPREPQV YTLPPSREEMTKNQVSLWCLVKGFYPSDIAVEWESNGQPENNYKTTPPVLDSDGSFFLY SKLTVDKSRWQQGNVFSCSVMHEALHNHYTQKSLSLSPG (SEQ ID NO:38) and a third polypeptide comprising the amino acid sequence of

(SEQ ID NO: 33)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

Multispecific antibodies have binding specificities for at least two different epitopes, usually from different antigens. Multispecific or bispecific antibodies can be prepared as full-length antibodies or antibody fragments (e.g., F(ab′)₂ bispecific antibodies).

To enable the targeted removal of a disease-causing agent via phagocytosis, an antigen-binding domain of the present disclosure may be selected from IgGs, intrabodies, peptibodies, nanobodies, single domain antibodies, SMTPs, and multispecific antibodies (e.g., bispecific antibodies, diabodies, triabodies, tetrabodies, tandem di-scFV, tandem tri-scFv, ADAPTIR).

Methods for making bispecific antibodies are known in the art. One well-established approach for making bispecific antibodies is the “knobs-into-holes” or “protuberance-into-cavity” approach. See e.g., U.S. Pat. No. 5,731,168. Two immunoglobulin polypeptides (e.g., heavy chain polypeptides) each comprise an interface; an interface of one immunoglobulin polypeptide interacts with a corresponding or cognate interface on the other immunoglobulin polypeptide, thereby allowing the two immunoglobulin polypeptides to associate. In some embodiments, interfaces may be engineered such that a “knob” or “protuberance” located in the interface of one immunoglobulin polypeptide corresponds with a cognate “hole” or “cavity” located in the interface of the other immunoglobulin polypeptide. In some embodiments, a knob may be constructed by replacing a small amino acid side chain with a larger side chain. In some embodiments, a hole may be constructed by replacing a large amino acid side chain with a smaller side chain. Knobs or holes may exist in the original interface, or they may be introduced synthetically. Polynucleotides encoding modified immunoglobulin polypeptides with one or more corresponding knob- or hole-forming mutations may be expressed and purified using standard recombinant techniques and cell systems known in the art. See, e.g., U.S. Pat. Nos. 5,731,168; 5,807,706; 5,821,333; 7,642,228; 7,695,936; 8,216,805; 8,679,785; 8,844,834; U.S. Pub. No. 2013/0089553; Spiess et al., Nature Biotechnology 31:753-758, 2013; and Ridgway and Carter (1996) Protein Eng. 9:617-621. Modified immunoglobulin polypeptides may be produced using prokaryotic host cells, such as E. coli, or eukaryotic host cells, such as mammalian cells (e.g., CHO cells) or yeast cells. Corresponding knob- and hole-bearing immunoglobulin polypeptides may be expressed in host cells in co-culture and purified together as a heteromultimer, or they may be expressed in single cultures, separately purified, and assembled in vitro. Exemplary cognate knob and hole mutations are provided below (numbering according to EU index). EU numbering as used herein is known in the art; see, e.g., IMGT resources at www.imgt.org/IMGTScientificChart/Numbering/Hu_IGHGnber.html and www.imgt.org/IMGTScientificChart/Numbering/Hu_IGKCnber.html. As used herein, an “antibody arm” may refer to the pairing between an antibody heavy chain and an antibody light chain, wherein the variable domains of the heavy and light chains form an antigen binding site that binds a target antigen.

| | | | | | | | | |
|---|---|---|---|---|---|---|---|---|
| Fc region 1 | Y407T | Y407A | F405A | T394S | T366S | T394W | T394S | T366W |
| | | | | | L368A | Y407T | Y407A | T394S |
| | | | | | Y407V | | | |
| Fc region 2 | T366Y | T366W | T394W | F405W | T366W | T366Y | T366W | F405W |
| | | | | | | F405A | F405W | Y407A |

According to a different approach, antibody variable domains with the desired binding specificities (antibody-antigen combining sites) are fused to immunoglobulin constant domain sequences.

In some embodiments, multispecific (e.g., bispecific) antibodies further comprise one or more mutations on only one of the antibody arms to improve heavy chain/light chain pairing. For example, amino acid substitutions can be used to replace a native disulfide bond in the CH1-CL interface of one antibody arm with an engineered disulfide bond. See, e.g., Mazor, Y. et al. (2015) MAbs 7:377-389 and EP3452089A2. In some embodiments, the multispecific or bispecific antibody comprises two antibody light chains and two antibody heavy chains, wherein only one of the antibody heavy chains comprises amino acid substitutions F126C and C220V, and only the corresponding or cognate light chain comprises amino acid substitutions S121C and C214V, according to EU numbering.

Multispecific (e.g., bispecific) antibodies also include cross-linked or “heteroconjugate” antibodies. Techniques for generating bispecific antibodies from antibody fragments have also been described in the literature. For example, bispecific antibodies can be prepared using chemical linkage. In some embodiments, a bispecific antibody comprises a first IgG antibody comprising the first antigen binding domain covalently linked to a second IgG antibody comprising the second antigen binding domain.

In some embodiments, multispecific (e.g., bispecific) antibodies further comprise one or more mutations on only one of the antibody arms to reduce binding affinity for Protein A. See, e.g., Ollier, R. et al. (2019) MAbs 11:1464-1478 and AU2018204314. In some embodiments, the multispecific or bispecific antibody comprises two antibody light chains and two antibody heavy chains, wherein only one of the antibody heavy chains comprises amino acid substitutions H435R and Y436F, according to EU numbering.

In some embodiments, the monospecific or multispecific (e.g., bispecific) antibodies further comprise one or more mutations to reduce effector function, e.g., to reduce or eliminate binding of the Fc region to an Fc receptor. In some embodiments, the antibody comprises two antibody Fc regions, wherein the antibody Fc regions comprise an amino acid substitution at one or more of positions 234, 235, and 237, according to EU numbering. In some embodiments, the antibody comprises two antibody Fc regions, wherein the antibody Fc regions comprise L234A, L235E, and G237A substitutions, according to EU numbering.

In some embodiments, the monospecific or multispecific (e.g., bispecific) antibodies comprise two antibody heavy chains and two antibody light chains, wherein the VH domain of the first antibody heavy chain forms an antigen binding domain with the VL domain of the first antibody light chain, wherein the VH domain of the second antibody heavy chain forms an antigen binding domain with the VL domain of the second antibody light chain, wherein the first antibody heavy chain comprises F126C, C220V, and T366W substitutions, wherein the first antibody light chain comprises S121C and C214V substitutions, and wherein the second antibody heavy chain comprises T366S, L368A, Y407V, H435R, and Y436F substitutions, according to EU numbering. In some embodiments, the first and second antibody heavy chains further comprise L234A, L235E, and G237A substitutions, according to EU numbering. In some embodiments, the first and second antibody heavy chains comprise human IgG1 Fc domains.

In some embodiments, provided herein is a polynucleotide encoding the antibody or multispecific binding protein of any one of the above embodiments. In some embodiments, provided herein is a vector (e.g., an expression vector) comprising the polynucleotide of any one of the above embodiments. In some embodiments, provided herein is a host cell (e.g., an isolated host cell or cell line) comprising the polynucleotide or vector of any one of the above embodiments. In some embodiments, provided herein is a pharmaceutical composition comprising the multispecific binding protein of any one of the above embodiments and a pharmaceutically acceptable carrier. Any of these may find use in the methods of production and/or treatment disclosed herein.

In some embodiments, provided herein is a method of producing a multispecific binding protein, comprising culturing the host cell of any one of the above embodiments under conditions suitable for production of the multispecific binding protein. In some embodiments, the method further comprises recovering the multispecific binding protein. The multispecific binding proteins may be produced using standard recombinant techniques, as described herein, and/or as exemplified infra.

Antibodies and antibody fragments may be produced using recombinant methods. For example, nucleic acid encoding the antibody/fragment can be isolated and inserted into a replicable vector for further cloning or for expression. DNA encoding the antibody/fragment may be readily isolated and sequenced using conventional procedures (e.g., via oligonucleotide probes capable of binding specifically to genes encoding the heavy and light chains of the antibody/fragment). Many vectors are known in the art; vector components generally include, but are not limited to, one or more of the following: a signal sequence, an origin of replication, one or more marker genes, an enhancer element, a promoter, and a transcription termination sequence. Suitable host cells for cloning or expressing the DNA in the vectors herein are the prokaryote, yeast, or higher eukaryote cells. When using recombinant techniques, the antibody/fragment can be produced intracellularly, in the periplasmic space, or directly secreted into the medium. If the antibody/fragment is produced intracellularly, the particulate debris, either host cells or lysed fragments, are removed, for example, by centrifugation or ultrafiltration. Where the antibody/fragment is secreted into the medium, supernatants from such expression systems are generally first concentrated using a commercially available protein concentration filter.

In some embodiments, a multispecific binding protein of the present disclosure is part of a pharmaceutical composition, e.g., including the antibody and one or more pharmaceutically acceptable carriers. Pharmaceutical compositions and formulations as described herein can be prepared by mixing the active ingredients (such as a fusion protein) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Certain aspects of the present disclosure relate to kits or articles of manufacture comprising any of the multispecific binding proteins disclosed herein. In some embodiments, the article of manufacture comprises a container and a label or package insert on or associated with the container. In some embodiments, the kit or article of manufacture further comprises instructions for using the multispecific binding protein according to any of the methods disclosed herein, e.g., for treating a disease or disorder such as cancer.

Suitable containers include, for example, bottles, vials, syringes, etc. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). At least one active agent in the composition is a multispecific binding protein as described herein. The label or package insert indicates that the composition is used for treating the particular condition. The label or package insert will further comprise instructions for administering the multispecific binding protein composition to the subject. Articles of manufacture and kits comprising combinatorial therapies described herein are also contemplated.

### II. Methods of Use

In certain aspects, the present disclosure provides methods of treating a disease or disorder, comprising administering an effective amount of an antibody, antibody fragment, multispecific (e.g., bispecific) binding molecule, or composition of the present disclosure to an individual in need thereof. In some embodiments, the individual is a human. In some embodiments, the individual has or has been diagnosed with cancer (e.g., non-Hodgkin's lymphoma or chronic lymphocytic leukemia) or an autoimmune condition relating to B cells (e.g., rheumatoid arthritis, systemic lupus erythematosus (SLE), multiple sclerosis, and Wegener's granulomatosis).

In some embodiments, the methods include using a multispecific (e.g., bispecific) binding molecule of the present disclosure with a first antigen binding domain that binds to human Dectin-1, and a second antigen binding domain that binds to a disease-causing agent, e.g., CD20 or hCD20. In some embodiments, the disease-causing agent is a B cell, tumor or cancer cell, e.g., a malignant B cell. In some embodiments, CD20 is expressed on the surface of a B cell, such as a malignant B cell. Binding of the molecule that mediates targeted removal of a disease-causing agent via phagocytosis could be with and without avidity i.e., with and without inducing dimerization of the phagocytosis receptor such as Dectin-1 or the target antigen present on the agent.

In addition to the beneficial removal of a disease-causing agent via phagocytosis, the molecule may induce production of inflammatory mediators to alter the disease microenvironment such as in tumors, cancers and lymphomas. Without wishing to be bound to theory, it is thought that the molecule that performs targeted phagocytosis may demonstrate clear benefits for patients such as cancer, inflammatory, or immune diseases (e.g., autoimmune diseases, inflammatory bowel diseases, multiple sclerosis), degenerative disease (e.g., joint and cartilage) Rheumatoid arthritis, Felty's syndrome, aggressive NK leukemia, IBM, IBD etc. In addition, targeted phagocytosis antibody treatment may have better activity of depleting cells in tissues over ADCC that relies on NK cells. The treatment may have a selective activity for removal of a particular disease-causing agent over a therapy that targets myeloid cells and improves phagocytosis in general. For example, targets of interest for treatment of cancer include CD20.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Functional Characterization of 2M24 Anti-Dectin-1 Antibody

This example describes the production of monoclonal antibodies specific for human Dectin-1. This example also describes the characterization of a novel anti-human Dectin-1 antibody.

### Materials and Methods

### Production of Anti-Dectin-1 Antibodies

Four-week-old, ATX-Gx transgenic mice were immunized subcutaneously with recombinant human Dectin-1 isoform B for five weeks, with one boost of antigen per week. Antibody titers in mouse serum were assessed during pre- and post-boosts via ELISA and flow cytometry. The mice with the highest serum antibody titer were selected to supply B cells for the generation of hybridomas.

Prior to cell fusion, mice were administered with one additional boost of recombinant human Dectin-1 isoform B. Mice were sacrificed and the spleens were harvested. Spleen cells and SP2/0-Ag14 myeloma cells were mixed, in which fusion was then induced by 37 C incubation and in the presence of polyethylene glycol (PEG) or electroporation. The cells were then harvested and plated into 96 well plates with limited dilution to achieve one cell per well. The cells were subsequently treated with hypoxanthine, aminopterin and thymidine (HAT) medium and selected for over 2 weeks in culture.

To identify candidates specific towards Dectin-1, the hybridoma supernatants were screened by flow cytometry on cells overexpressing Dectin-1 and human primary monocytes. Cynomolgus monkey Dectin-1 cross-reactivity was assessed by antibody binding to cynomolgus monkey primary monocytes using flow cytometry.

### Healthy Donor Samples

Fresh healthy donor buffy coats were obtained from Stanford Blood Center Peripheral blood mononuclear cells were isolated via ficoll paque (GE Healthcare, Chicago, IL) separation and cryopreserved in Bambanker cell freezing media (Bulldog Bio, Portsmouth, NH). Briefly, buffy coats were diluted in phosphate buffered saline (in 1:1 ratio), followed by layering of the diluted buffy coat in ficoll and centrifugation at 760 g. The PBMC layer was isolated and washed in PBS prior to downstream analysis Peripheral blood leukocytes were isolated through red blood cell lysis Cryopreserved cynomolgus monkey PBMC were obtained from Human Cells Biosciences.

### Primary Cells and Cell Culture

Human monocytes were isolated from healthy donor PBMCs according to the manufacturer's instructions of the pan monocyte isolation kit (Miltenyi Biotec, Inc., Auburn, CA) For macrophage and dendritic cells differentiation, monocytes were cultured in RPMI with 10 Human Serum (Millipore Sigma) in the presence of 50 ng/ml MCSF (Peprotech, Rocky Hill, NJ) for 6 days to fully differentiate into macrophages or in the presence of 50 ng/ml GMCSF and 50 ng/ml IL-4 (Peprotech, Rocky Hill, NJ) for 6 days to fully differentiate into dendritic cells. The medium with cytokines was refreshed every 3 days.

HEK Blue hDectin-1-a cells and HEK Blue hDectin-1-b cells (Invivogen, San Diego, CA) were maintained in DMEM/10% FBS supplemented with mormocin and puromycin according to manufacturer's instructions. Freestyle 293F cells were transiently transfected according to the manufacturer's suggestion (Thermo Fisher, Waltham, MA) Briefly, viable cell density and percent viability was determined Cells were diluted to a final density of 11×10⁶ viable cells/mL with Freestyle 293 Expression Medium. Freestyle Max Reagent was diluted with OptiPro SFM Medium, mixed and incubated at room temperature for 5 minutes. The diluted Freestyle Max Reagent was added to plasmid DNA diluted with OptiPro SFM Medium and mixed. The Freestyle Max Reagent/plasmid DNA complexes were incubated at room temperature for 10-20 minutes. The complexes were slowly transferred to the cells, swirling the culture flask gently during the addition, and the cells were then incubated in a 37° C. incubator with 80% relative humidity and 8% CO2 on an orbital shaker.

### Binding of Dectin-1 Antibodies to Dectin-1 Expressing Cells

Dectin-1 expressing cells (HEK Blue hDectin-1-a, HEK Blue hDectin-1-b, HEK293F hDectin-1 a FL, human monocytes or cyno monocytes) were plated at 1×10⁵-2×10⁵ cells per well in non-tissue culture treated, 96 well V bottom plates. Additionally, human monocytes were incubated in human FcgR blocking antibody (Biolegend, San Diego, CA) for 10 minutes at room temperature to reduce binding of the antibodies to the Fc receptor. The cells were subsequently stained with the eFluor 506 viability dye (ThermoFisher, Waltham, MA) in a 1:1000 dilution for 30 minutes on ice, followed by a wash step in FACS buffer (PBS with 2% fetal bovine serum). Primary Dectin-1 antibodies or isotypes were used at a titration of 300, 100, 33.3, 11.1, 3.7, 1.23, 0.41, and 0.14 nM and incubated on ice for 30 minutes, followed by another wash step in FACS buffer.

For detection of mouse primary antibodies, the cells were incubated with a fluorescently labeled AF647 anti-mouse Fc-specific secondary antibody (Jackson Immuno). For detection of human IgG4 primary antibody, the cells were incubated for 30 minutes on ice with an Alexa Fluor 647 anti-human Fc-specific secondary antibody (Jackson Immuno) (detection in HEK cells) or a FITC anti-human IgG4 antibody (Sigma) (detection in primary monocytes). Data acquisition was performed using a CytoFlex flow cytometer (Beckman Coulter, Atlanta, GA) and analyzed using Graphpad Prism 8.4.

### Dectin-1 Antibody Blocking of Laminarin

HEK Blue hDectin-1a cells were plated at 1×10⁵ cells per well in non-tissue culture treated, 96 well V bottom plates. Primary anti-Dectin-1 antibodies were used at a titration of 300, 100, 33.3, 11.1, 3.7, 1.23, 0.41, 0.14, 0.05, 0.015 and 0.005 nM and incubated on ice for 30 minutes in the presence of 8 μg/ml biotin laminarin. Following a wash step in FACS buffer, binding of biotin laminarin on the HEK cells was detected using streptavidin-AF647 for 30 minutes on ice. For analysis, 4000 cell events were acquired in a CytoFlex flow cytometer (Beckman Coulter, Atlanta, GA) and analyzed using Graphpad Prism 8.4.

Labelling of Polystyrene Beads with pHrodo and Conjugation to Antibodies

Polystyrene beads of different sizes coated with goat anti-mouse IgG (or biotin) (Spherotech, Lake Forest, IL) were washed with PBS/Tween20 0.05% twice. pHrodo Red, succinimidyl ester (pHrodo Red, SE) (ThermoFisher, Waltham, MA) was added to the beads at 10 μM and allowed to incubate for 60 minutes at room temperature with shaking. The beads were then washed with PBS/BSA 0.1% to remove excess pHrodo Red.

After pHrodo labeling, the antibody was conjugated to the beads according to the manufacturer's recommendations. Briefly, based on the binding capacity of the beads to antibody, an 5× excess of antibody was added to the beads and allowed to incubate at room temperature for 60 minutes with shaking. The beads were then washed with PBS/BSA 0.1% to remove unbound antibody. To assess the quality of the beads, pHrodo red activation was assessed in low pH buffer by flow cytometry. Antibody bound on the beads was assessed using a fluorescently labeled AF647 anti-mouse Fc specific or a FITC anti-human IgG4 antibody secondary antibody.

### Antibody-Dependent Targeted Phagocytosis of Phrodo Labeled Beads

For phagocytosis experiments, 50,000 HEK cells overexpressing Dectin-1 or primary cells (macrophages or dendritic cells) were seeded in a 96-well plate in RPMI with 10 ultra-low IgG FBS. pHrodo-labelled beads conjugated to anti-Dectin-1 antibodies or isotypes were added at a desired ratio ranging from 1:1 to 1:3 cells beads, and the plates were briefly spun down.

In some experiments cell tracker Calcein AM (Thermo Fisher, Waltham, MA) was added to label the cells. Phagocytosis was monitored in an IncuCyte S3 live imaging system (Germany) by taking images at desired time points and analyzed using the IncuCyte S3 software. Phagocytosis was quantified as the overlap of bright red fluorescence (engulfed beads) with Calcein AM positive cells or integrated red intensity of bright red fluorescence.

SEAP Reporter Assay in HEK Cells Overexpressing Dectin-1 with Anti Dectin-1 Antibodies

Anti-Dectin-1 monoclonal antibodies 2M24 (VH and VL domains comprising SEQ ID NO: 7 and 8, respectively) or 15E2 and control isotypes were immobilized by coating onto the surfaces of wells of untreated 96-well, U bottomed polypropylene microtiter plates. For coating, 10, 2, 1, 0.5 and 0.1 μg of the anti-Dectin-1 antibody diluted in 50 μl sterile PBS was added to each well. Plates were left overnight in a class II laminar flow cabinet with the lids removed to allow the solutions to evaporate. Coated plates were washed twice with 200 μl sterile PBS to remove salt crystals and unbound antibody. HEK Blue hDectin-1-a cells were then cultured on the plates in RPMI with 10% ultra-low IgG FBS (VWR) for 22 hours and alkaline phosphatase levels were assessed in the supernatant at OD 630 nm using QUANTI Blue Solution (Invivogen, San Diego, CA) per manufacturer's instructions.

To determine HEK cells SEAP secretion induced by anti-Dectin-1 antibody conjugated beads, streptavidin-2M24 (hIgG4) was conjugated to biotin polystyrene beads of 3, 10 and 16 μm in size (Spherotech, Lake Forest, IL) by incubating the beads with the antibody for 30 minutes in room temperature and washing twice with PBS to remove the unbound antibody. Anti-Dectin-1 antibody-conjugated beads were mixed with 1×10⁵ HEK Blue hDectin-1-a cells at a ratio of 1:3 cells: bead in RPMI with 10% ultra-low IgG FBS for 22 hours, followed by evaluation of alkaline phosphatase secretion at OD 630 nm in the supernatant as described above.

### Cytokine Secretion

Anti-Dectin-1 monoclonal antibodies 2M24 or 15E2 clones and control isotypes were immobilized by coating 10 ug onto the surfaces of wells of untreated 96-well, U bottomed polypropylene microtiter plates as described above. Freshly isolated monocytes or peripheral blood mononuclear cells were then cultured on the plates with the immobilized antibodies in RPM1 with 10% ultra-low IgG FBS at 200,000 cell/per well for 24 hours. In other wells the cells were treated with 10 μg/ml of Dectin-1 antibodies in solution instead of immobilized antibodies. TNFa, IL-6 and IFNg in the supernatant were assessed using the U-PLEX Assay Platform (Meso Scale Discovery) and their levels were expressed as fold change of Dectin-1 antibody-induced cytokine secretion versus the isotype control. As a positive control, cells were stimulated with zymosan at 25 μg/ml.

### Results

To generate Dectin-1 antibodies, four-week-old, ATX-Gx Alloy transgenic mice were immunized subcutaneously with recombinant Dectin-1 isoform B protein, with one boost of antigen per week. The antibodies generated from this immunization have a human variable domain and a mouse constant domain.

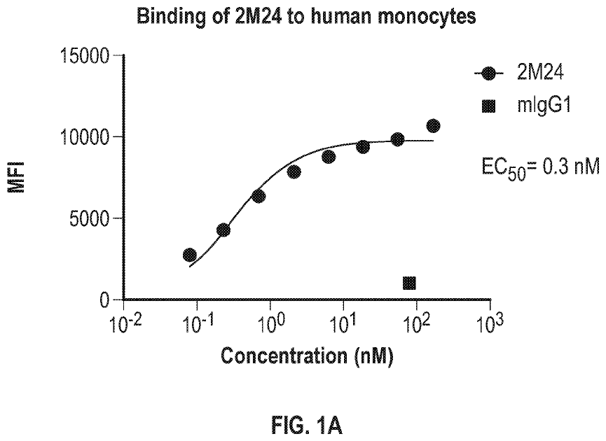

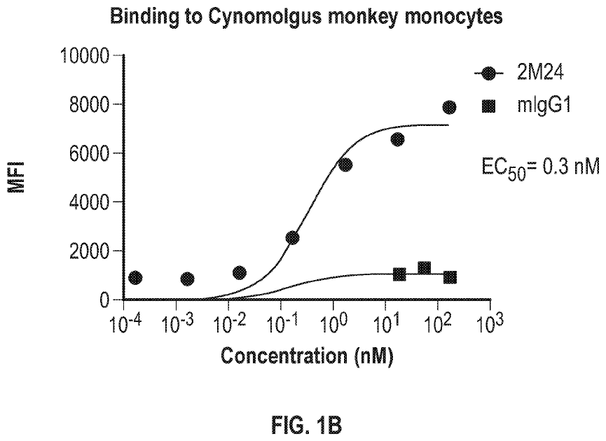

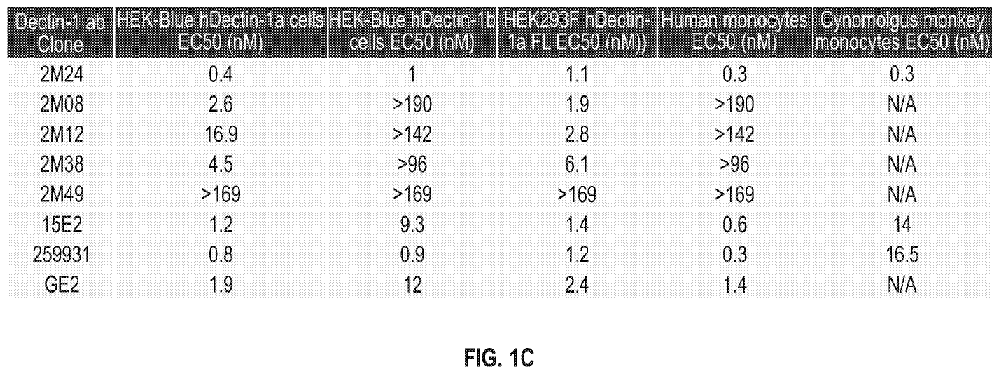

From the 56 candidate anti-Dectin-1 antibody clones generated in this study, the 2M24 clone was the only one that showed binding to both Dectin-1 isoforms A and B in HEK cells as well as to monocytes. As shown in FIG. 1A, the 2M24 anti-Dectin-1 antibody clone demonstrated high affinity to Dectin-1 expressing human monocytes. In contrast, other clones bound only to Dectin-1 isoform A (e.g., 2M08, 2M12, 2M38) or showed no binding at all (2M49). Moreover, the affinity to Dectin-1 of 2M24 was superior to the affinity presented by other clones and the commercial Dectin-1 antibodies (15E2, 259931, GE2). FIG. 1C shows a comparison of the binding to human monocytes and HEK cells overexpressing Dectin-1 between 2M24 clone and other Dectin-1 clones identified from the Alloy transgenic mice immunization, as well as commercial Dectin-1 clones.

The 2M24 antibody was also assessed for its cross-reactivity to cynomolgus Dectin-1. The binding was assessed by flow cytometry analysis of cynomolgus monkey monocytes derived from PBMCs. As shown in FIG. 1B, anti-human Dectin-1 clone 2M24 antibody demonstrated cross-reactivity and high affinity to cynomolgus monkey Dectin-1 expressed on monocytes. The 2M24 anti-Dectin-1 antibody was superior to commercial antibodies tested in terms of affinity, exhibiting an EC50 of 0.3 nM. The agonistic 15E2 and the 255931 commercial antibodies exhibited EC50 of 14 nM and 16 nM, respectively, in cynomolgus monkey monocytes. FIG. 1C shows a comparison of binding to cynomolgus monkey monocytes between 2M24 clone and the commercial clones 15E2 and 259931.

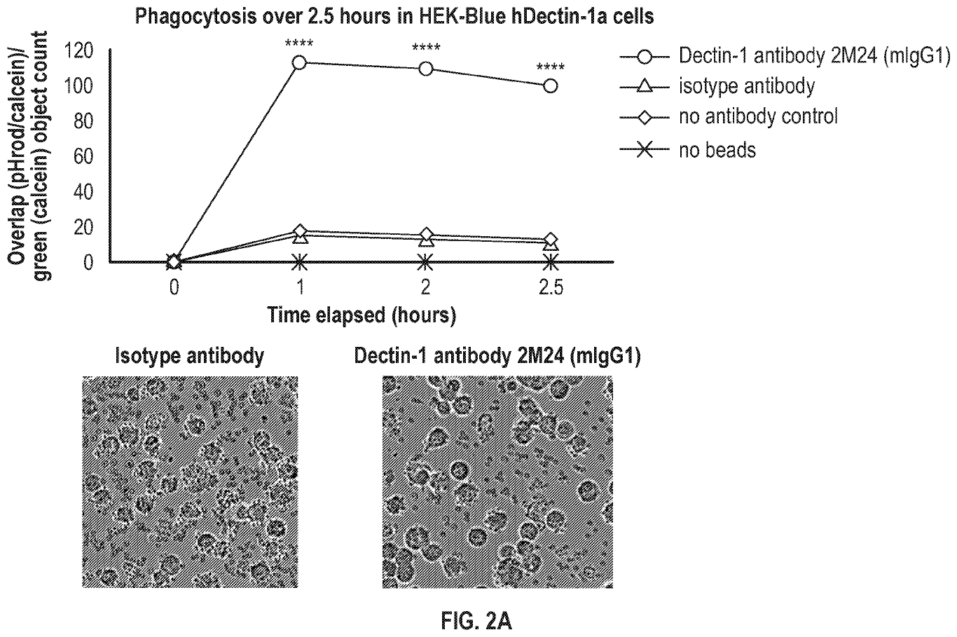

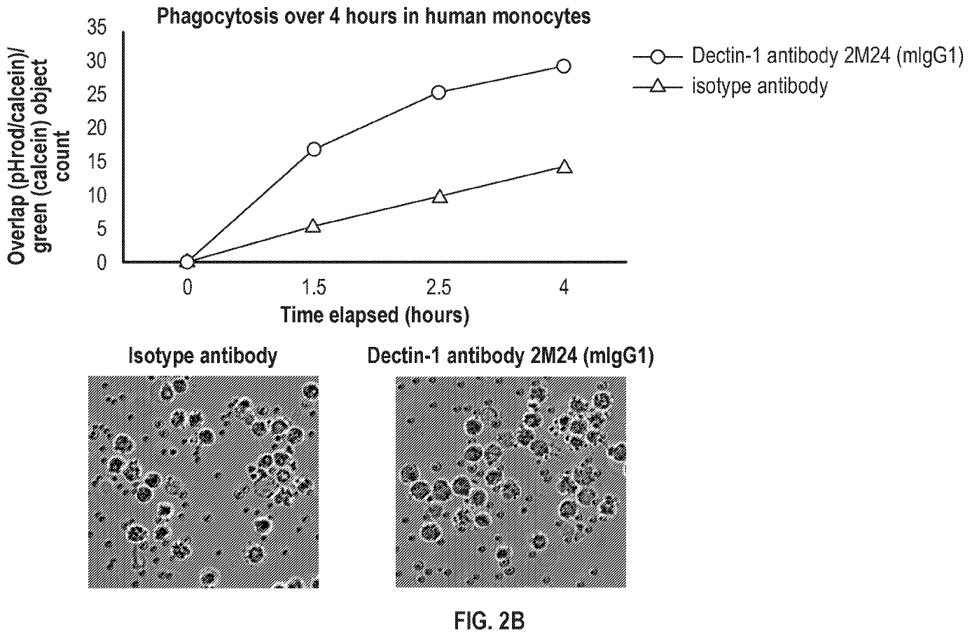

To assess the functionality of the 2M24 Dectin-1 antibody in promoting phagocytosis, polystyrene beads were coated with the 2M24 antibody and mixed with HEK-Blue hDectin-1a cells or primary human monocytes. The 2M24 antibody efficiently induced the phagocytosis of the beads. As shown in FIGS. 2A-2B, the 2M24 anti-Dectin-1 antibody coupled to polystyrene beads promoted phagocytosis in both HEK-Blue hDectin-1a cells and human primary monocytes.

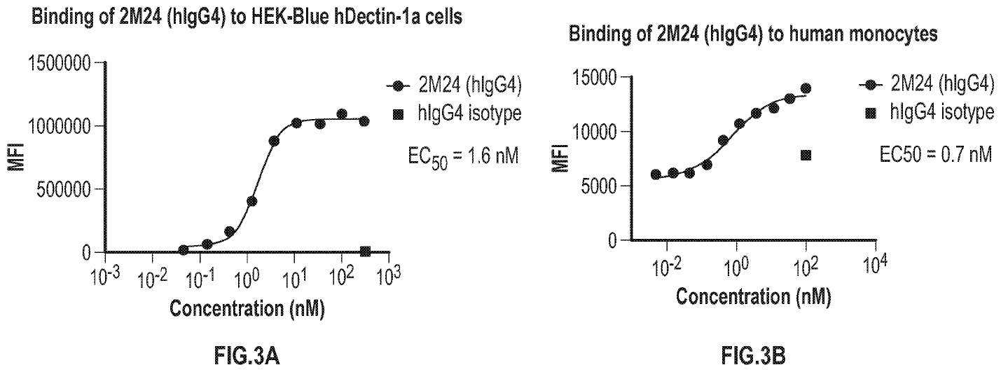

From the mIgG1 2M24 clone, a fully human 2M24 antibody of the IgG4 isotype was developed. This antibody has human constant and variable regions. The functionality of the hIgG4 2M24 was then assessed for binding to two Dectin-1 expressing cell types, HEK-Blue hDectin-1a cells and human monocytes. As shown in FIGS. 3A-3B, the fully human 2M24 showed high affinity binding to Dectin-1 in transfected HEK cells (EC50=1.6 nM) and human monocytes (EC50=0.7 nM).

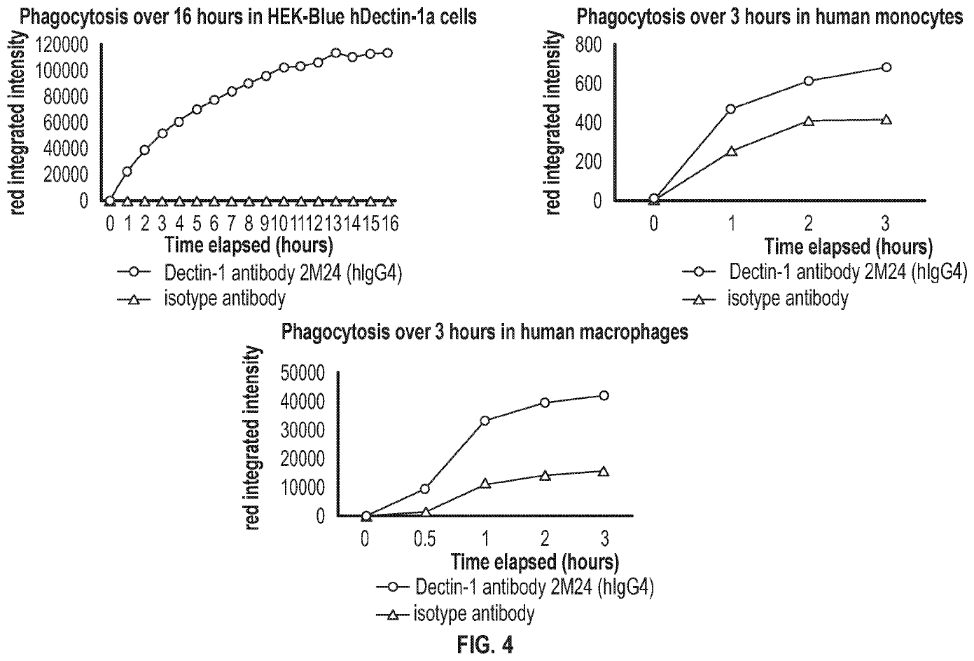

Next, the hIgG4 2M24 antibody was tested for its ability to promote phagocytosis of beads in Dectin-1 expressing cells. As shown in FIG. 4, the hIgG4 2M24 antibody exhibited efficient phagocytic ability in HEK cells overexpressing Dectin-1, human monocytes, and human macrophages. Thus, the fully human IgG4 2M24 antibody can promote phagocytosis in Dectin-1 expressing cells.

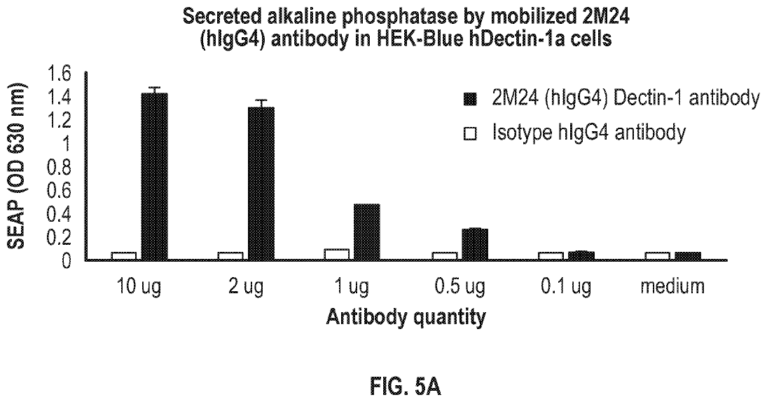

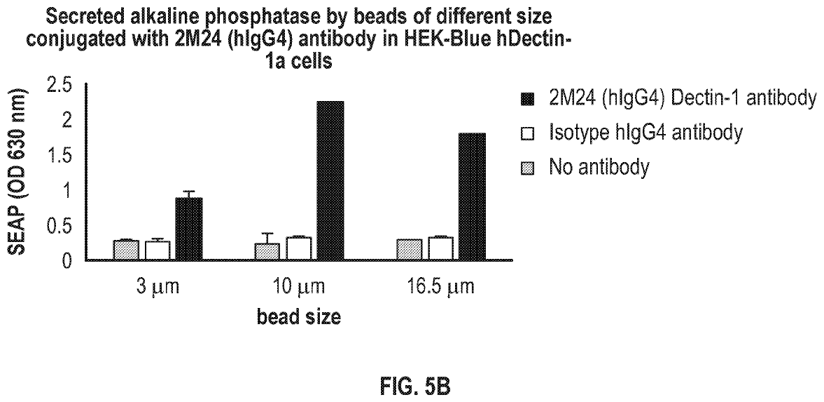

The fully human 2M24 (hIgG4) anti-Dectin-1 antibody was also tested for its ability to promote signaling through Dectin-1. Activation of Dectin-1 signaling by the antibodies can be assessed with a secreted alkaline phosphatase assay using HEK-Blue hDectin-1a cells. The HEK-Blue hDectin-1a cells have been engineered to express Dectin-1 isoform A and genes involved in the Dectin-1/NF-κB/SEAP signaling pathway and thus express a secreted alkaline phosphatase (SEAP) in response to stimulation by Dectin-1 ligands. As shown in FIGS. 5A-5B, the 2M24 (hIgG4) anti-Dectin-1 antibody induced alkaline phosphatase secretion in HEK-Blue hDectin-1a cells both in immobilized form and conjugated to beads. These observations support the idea that the 2M24 (hIgG4) antibody promotes SEAP secretion by engaging Dectin-1 on the surface of the cells, indicating clustering of the receptor and an agonistic activity by this antibody. Moreover, efficient clustering signaling of Dectin-1 can be promoted by beads conjugated to 2M24 (hIgG4). Signaling was better induced with bigger beads, reflecting better clustering of the receptor. This supports that clustering of Dectin-1 promoted by a bispecific antibody comprising the anti-Dectin-1 antibody, which targets a phagocyte, and an antibody targeting another cell, such as a cancer cell, could promote clustering and signaling by Dectin-1 on the phagocyte.

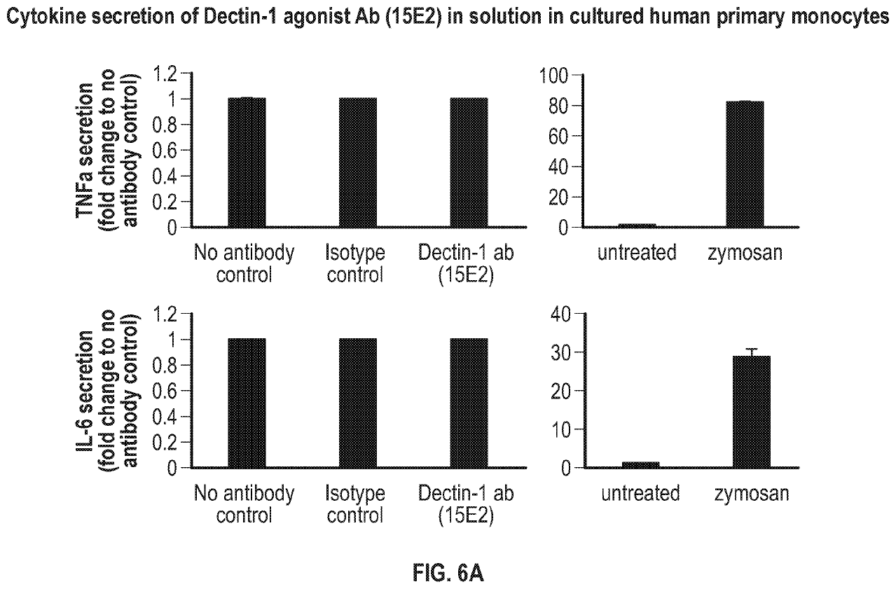

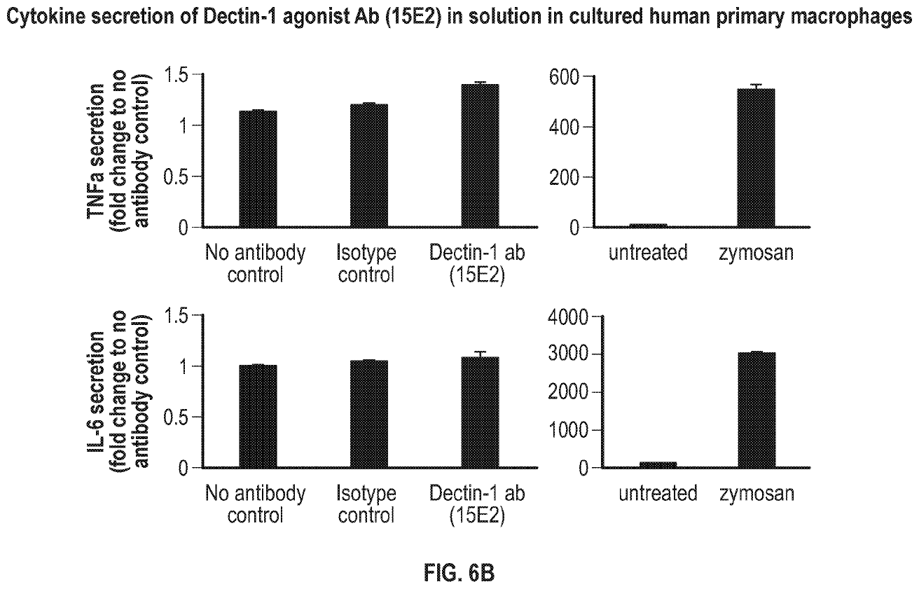

Natural ligands of Dectin-1 cluster the receptor and signal downstream of Dectin-1/Syk/NFkB to induce inflammatory gene expression. To assess if engagement of Dectin-1 antibody in solution can trigger cytokine secretion, monocytes or macrophages were treated with 10 ug/ml of a commercial anti-Dectin-1 antibody. As shown in FIGS. 6A-6B, the 15E2 commercial anti-Dectin-1 antibody did not induce cytokine secretion in primary human macrophages and monocytes, indicating that there was insufficient clustering of the Dectin-1 receptor. This data supports that free Dectin-1 antibody in solution does not induce immunostimulation, due to lack of sufficient Dectin-1 clustering.

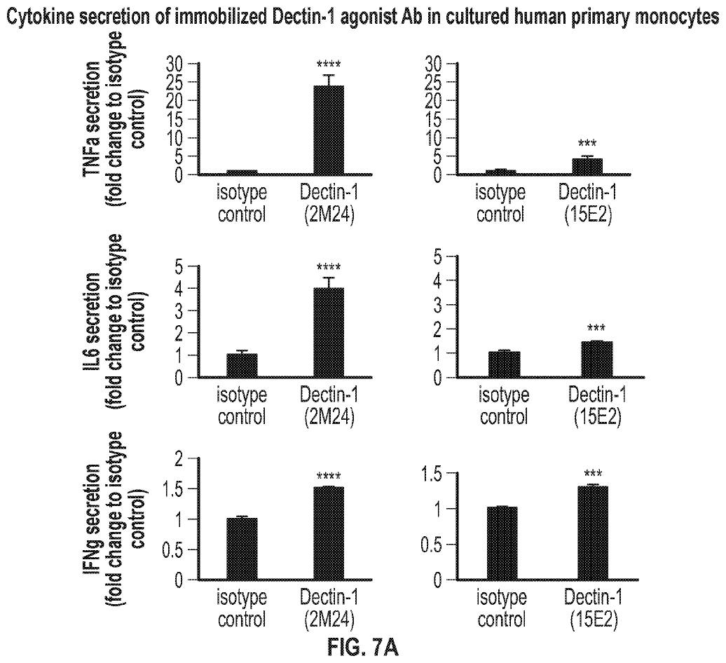

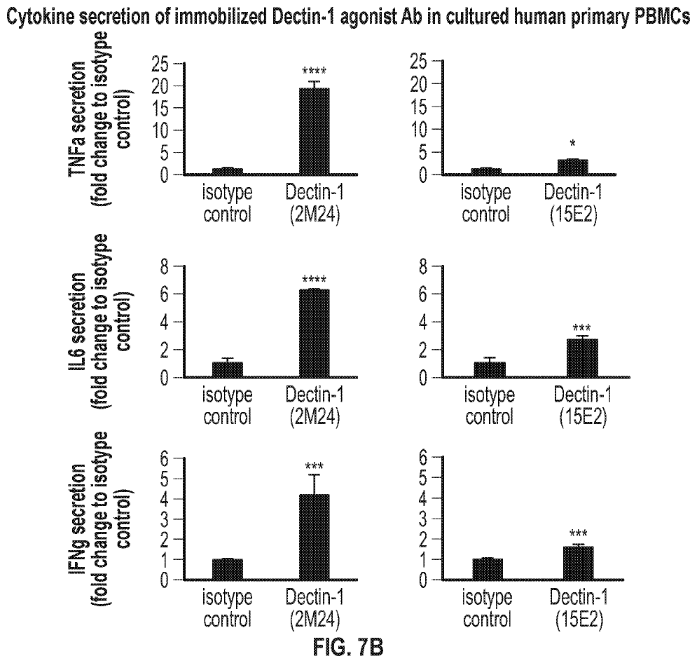

To assess if cytokine secretion could be induced by the 2M24 (hIgG4) anti-Dectin-1 antibody, the antibody was immobilized on beads and cultured with monocytes or PBMCs. As shown in FIGS. 7A-7B, the 2M24 anti-Dectin-1 antibody induced cytokine secretion in primary human monocytes and PBMCs. The 2M24 antibody not only promoted cytokine secretion, but also exhibited superior immunostimulation as compared to that promoted by the 15E2 anti-Dectin-1 agonistic antibody. Of the cytokines measured in this experiment, TNFa and IL6 are secreted by monocytes that express Dectin-1. In contrast, IFNg is mainly secreted by T-cells that exist in PBMCs. Because T-cells do not express Dectin-1, they are not activated directly by the anti-Dectin-1 antibodies, but rather from cytokines secreted by the monocytes in the PBMCs that are stimulated by the Dectin-1 antibodies. The differential effect of Dectin-1 antibodies on IFNg was therefore more prominent in PBMCs than in pure monocytes.

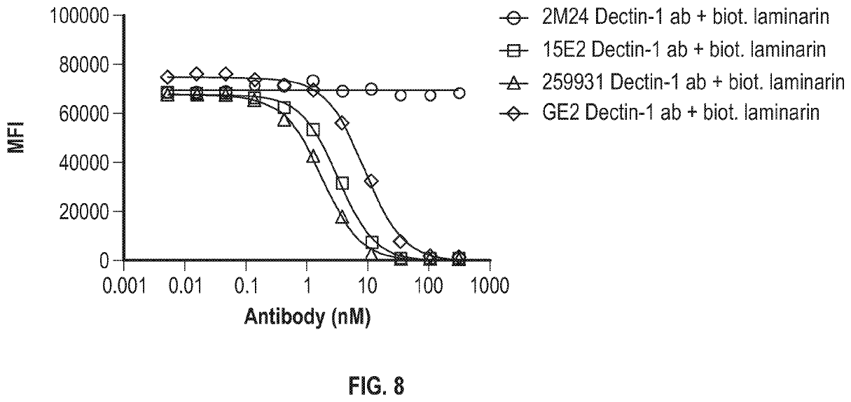

Finally, the activation of Dectin-1 by natural ligands in the presence of anti-Dectin-1 antibody was tested. HEK-Blue hDectin-1a cells were incubated in a ⅓ serial dose titration of 2M24 (hIgG4) Dectin-1 antibody or the 15E2, 259931, GE2 anti-Dectin-1 commercial antibodies starting at 300 nM in the presence of 8 ug/ml of biotinylated laminarin. As shown in FIG. 8, binding of the 2M24 (hIgG4) antibody to Dectin-1 did not block the binding of laminarin, a natural ligand of Dectin-1. Thus, engaging Dectin-1 with the 2M24 anti-Dectin-1 antibody does not block clearance of pathogens and is unlikely to increase susceptibility to potential fungal infections.

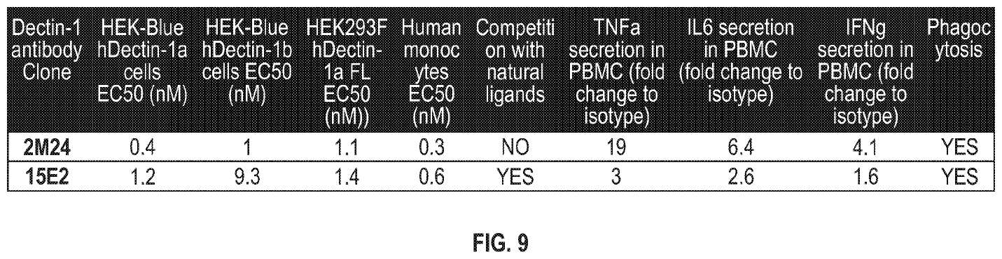

In conclusion, the 2M24 anti-Dectin-1 antibody can induce phagocytosis by Dectin-1 expressing cells and can induce activation of Dectin-1 signaling without competing with the natural ligands for Dectin-1. The properties of the 2M24 and 15E2 antibodies are summarized in FIG. 9.

### Example 2: Bispecific Anti-Dectin-1 Antibodies

This example describes the generation and characterization of bispecific antibodies comprising a Dectin-1-binding arm and a second arm that binds specific tumor antigens.

### Materials and Methods

### Generation of Bispecifics

Antibodies were differentially labeled with MTA or FOL reagent following manufacturer's guidelines (AAT Bioquest). Labeled antibodies were mixed and incubated to allow for covalent assembly via MTA and FOL interaction. The following antibodies were used for biotin: streptavidin-induced bispecific antibodies:

Anti-Dectin-1 15E2 antibody heavy chain: mSA
fusion
(SEQ ID NO: 18)
QWQLQQSGAELARPGASWKMSCKASGYTFTTYTMHWWKQRPGQGLEWIGY
INPSSGYTNYNQKFKDKATLTADKSSSTASMQLSSLTSEDSAWYYCARER
AVLVPYAMDYWGQGTSVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVGGGSGGGSGGGSEFASAEAGITGTWYNQHGS
TFTVTAGADGNLTGQYENRAQGTGCQNSPYTLTGRYNGTKLEWRVEWNNS
TENCHSRTEWRGQYQGGAEARINTQWNLTYEGGSGPATEQGQDTFTKVKP
SAASGSAAAGASHHHHHH
Anti-Dectin-1 15E2 antibody light chain
(SEQ ID NO: 19)
QIVLTQSPAVMSASPGEKWTITCTASSSLSYMHWFQQKPGTSPKLWLYST
SILASGVPTRFSGSGSGTSYSLTISRMEAEDAATYYCQQRSSSPFTFGSG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Avi-tagged anti-CD20 Fab heavy chain (CH1 domain
based on hIgG4 sequence)
(SEQ ID NO: 20)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVAAAGASHHHHHHGSGLNDIFEAQKIEWHE
Anti-CD20 Fab light chain
(SEQ ID NO: 21)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Avi-tagged anti-HER2 Fab heavy chain (CH1 domain
based on hIgG4 sequence)
(SEQ ID NO: 22)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVAAAGASHHHHHHGSGLNDIFEAQKIEWHE
Anti-HER2 Fab light chain
(SEQ ID NO: 23)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### Cell Coupling Assay

Dectin-1-expressing cells were labelled with calcein green, and target cells were labelled with calcein reds. The cells were incubated in the presence of a bispecific or an isotype control antibody, then analyzed by flow cytometry. Coupling of the cells was indicated by a double positive signal (green+red+). Coupling efficiency was quantified as the percentage of total target cells that forms doublets with Dectin-1-expressing cells.

Five million effector (Dectin-1 expressing cells) or target cells (cells expressing the target of interest, e.g., CD20 positive Raji cells or HER2 positive SKBR3 cells) were differentially labeled with either calcein green (0.5 nM) or calcein red/pHrodo-red (0.5 nM). Cells were thoroughly washed with PBS and kept on ice. Effector and target cells were then co-cultured at a 3:1 ratio (effector:target) in the presence of 2M24 bispecific antibody or isotype control and incubated for 30 minutes at 37° C. Following incubation, samples were gently resuspended and analyzed by flow cytometry. PMT voltages were adjusted accordingly, and cells were gated based on FITC and/or PE fluorescence corresponding to calcein green or red fluorescence. Coupling efficiency is reported as the number of PE-positive cells (target cells) in the doublet population, divided by the total number of PE-positive target cells in the reaction.

SEAP Reporter Assay in HEK Cells Overexpressing Dectin-1 with Anti Dectin-1 Antibodies

To determine HEK cell SEAP secretion induced by Raji cells (expressing CD20), Raji cells were coated with a 2M24/anti-hCD20 or a hIgG4/anti-CD20 bispecific for 30 minutes on ice, followed by washing twice with PBS to remove the unbound bispecific. The bispecific-coated Raji cells were mixed with 1×10⁵ HEK Blue hDectin-1-a cells at a ratio of 1:2 (HEK cells Raji cells) in RPM1 with 10% ultra-low IgG FBS. After 22 hours, alkaline phosphatase secretion in the supernatant was evaluated at OD 630 nm as described in Example 2.

### Results

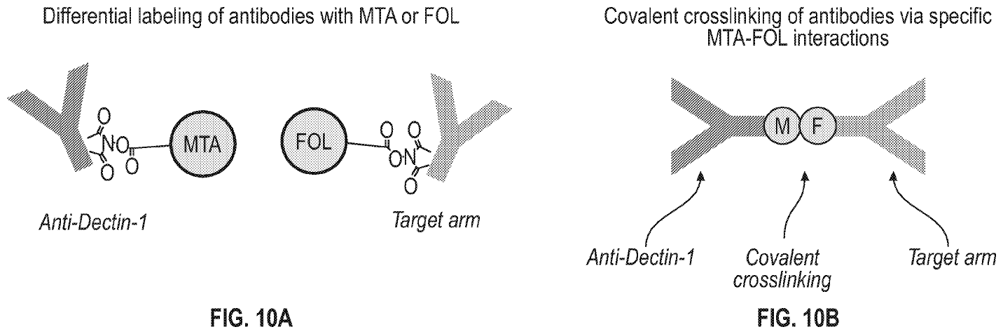

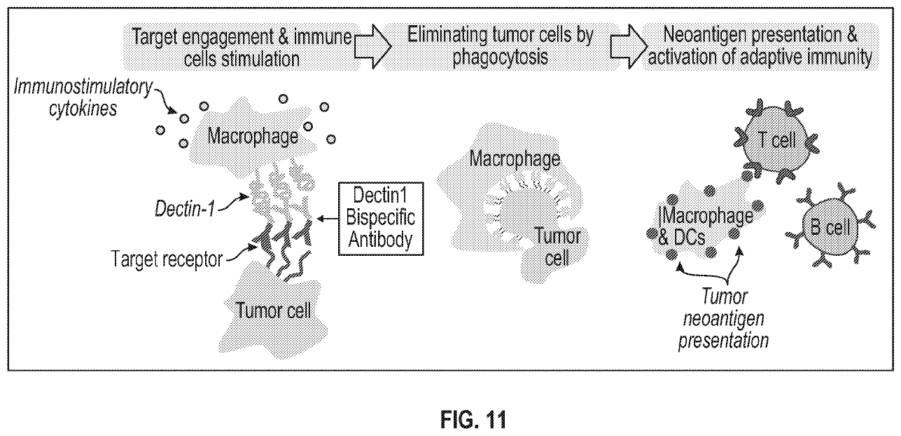

Dectin-1 agonist bispecific antibodies can exploit various modes of activity (e.g., immune activation, phagocytosis, neoantigen presentation and adaptive immunity activation) for the targeted depletion of cancer cells (FIG. 11). As a proof-of-concept to engage a Dectin-1 antibody (15E2 or 2M24) and a target antibody, a click-chemistry approach was used to develop bispecifics comprising an anti-Dectin-1-targeting arm and a second arm targeting a protein of interest. This approach enabled the generation of bispecifics for various assays. A schematic of this approach is shown in FIGS. 10A-10B. As binding of Dectin-1 by Dectin-1-specific antibodies can induce phagocytosis of targets (see Example 1 and Example 2), the bispecific antibodies were evaluated for their ability to promote phagocytosis of specific target cells. First, the bispecifics were evaluated for their ability to eliminate CD70-expressing cancer cells by phagocytosis. CD70 is a type II transmembrane glycoprotein that belongs to the tumor necrosis factor (TNF) superfamily. CD70 is expressed at low levels in normal tissues, but is highly overexpressed in various diseases, including acute myeloid leukemia (AML), renal cell carcinoma, rheumatoid arthritis and lupus.

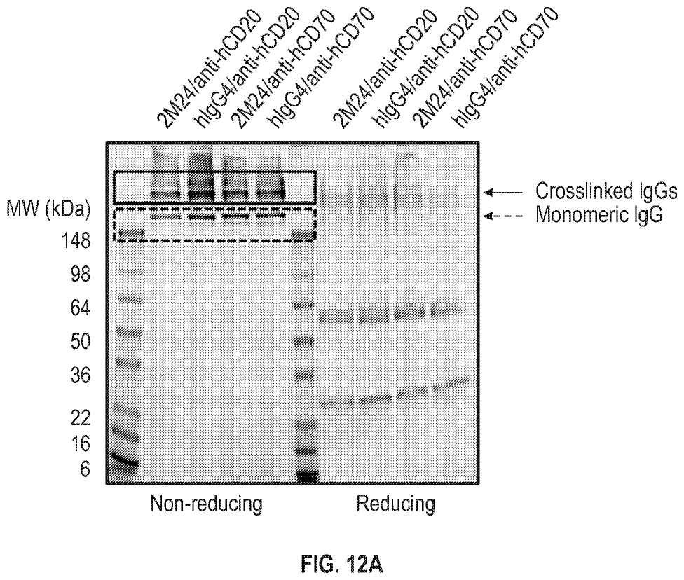

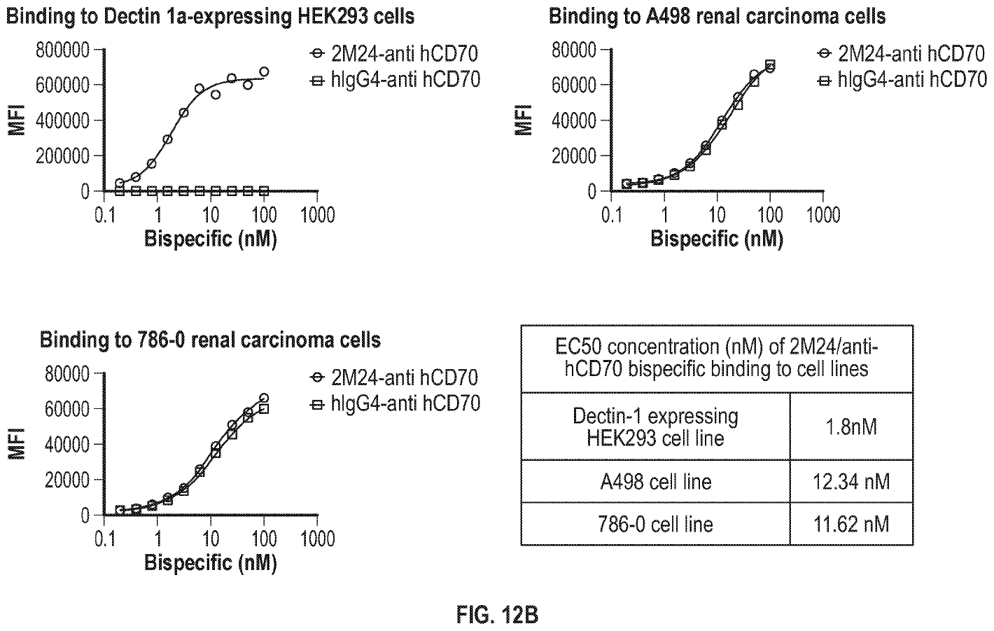

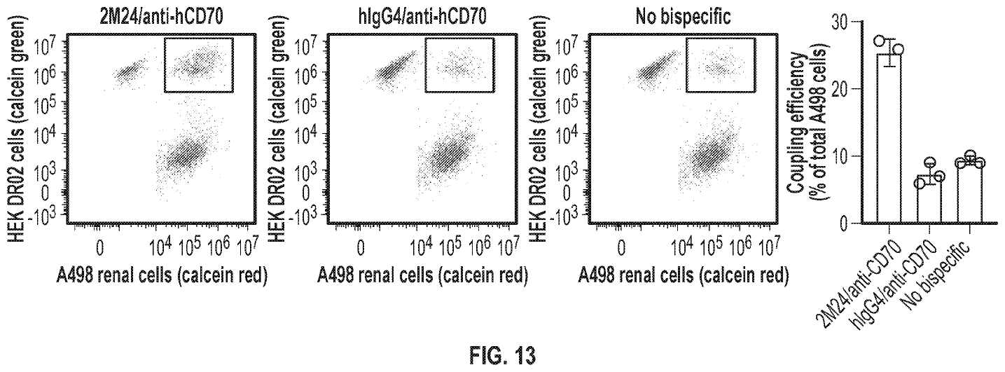

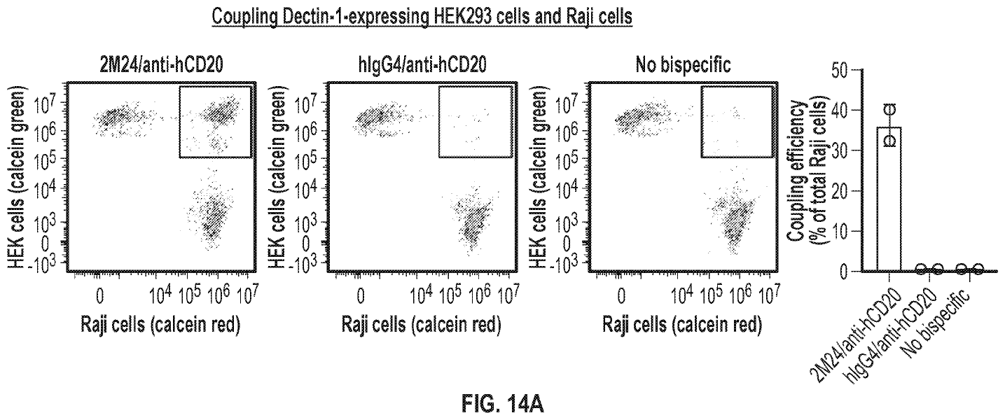

Using click chemistry, a bispecific molecule comprising a Dectin-1-targeting arm (anti-Dectin-1; clone 2M24) and a CD70-targeting arm (anti-hCD70; clone 113-16) was generated. The purity of the bispecific (2M24/anti-hCD70) antibody was assessed by SDS-PAGE analysis (FIG. 12A), while binding was assessed by flow cytometry analysis (FIG. 12B). As shown in FIG. 12B, binding studies on cells revealed that 2M24/anti-hCD70 bound to the HEK293 cell line expressing Dectin-1 with an EC50 of 1.8 nM and bound to CD70-positive renal carcinoma cell lines with an EC50 of 12.34 nM (A498 cells) or 11.62 nM (786-0 cells). The bispecific was then evaluated for its ability to induce cell coupling. As shown in FIG. 13, the 2M24/anti-hCD70 bispecific induced coupling of Dectin-1-expressing HEK293 cells and CD70-expressing renal carcinoma cells, resulting in cell doublets of HEK293 cells (labeled with calcein green) and A498 cells (labeled with calcein red).

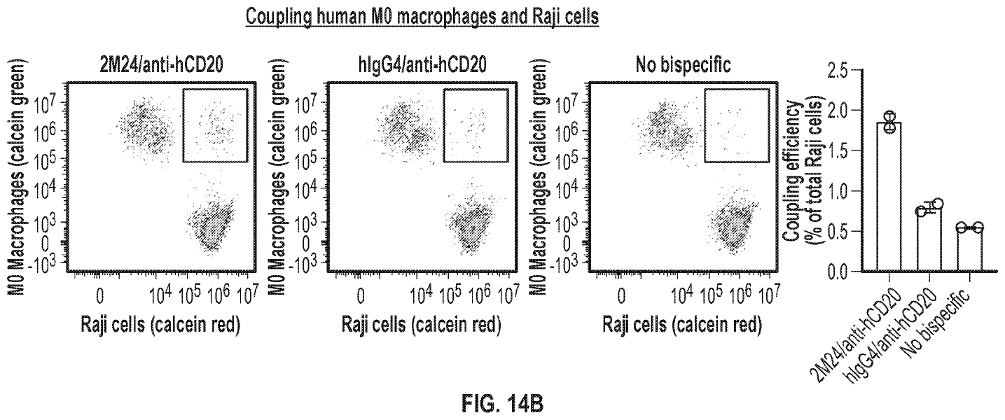

Next, targeting of CD20-expressing cells with a bispecific was evaluated. CD20 is a transmembrane protein present on virtually all B cells from the stage at which they become committed to B-cell development until it is downregulated when they differentiate into antibody-secreting plasma cells and is considered a pan-B-cell antigenic marker. As shown in FIGS. 14A-14B, a 2M24/anti-hCD20 bispecific induced coupling of Dectin-1-expressing cells (both Dectin-1-expressing HEK293 cells and human M0 macrophages) with CD20-expressing B cells (Raji cell line). This cell-to-cell coupling mediated by the bispecific could induce synapse formation between effector and target cell that may alter cytokine signaling, activate phagocytosis and ultimately target antigen presentation.

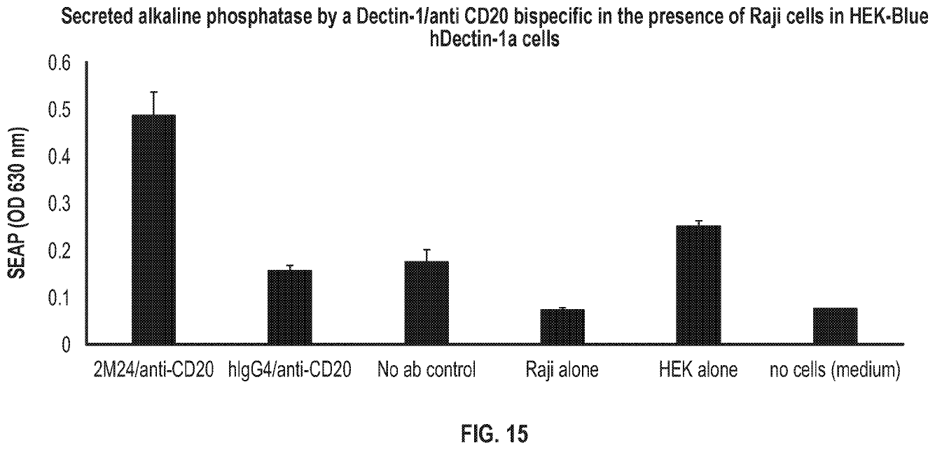

To test for induction of signaling resulting from stimulation with bispecific antibodies that bind Dectin-1, a secreted alkaline phosphatase assay was performed. As shown in FIG. 15, Raji cells coated with an anti-Dectin-1/anti-CD20 bispecific induced alkaline phosphatase secretion in HEK-Blue hDectin-1a cells. Thus, using a bispecific antibody to connect a target cell with a cell expressing Dectin-1 (such as phagocyte) can promote signaling by the Dectin-1 expressing cell. In the case of phagocytes, signaling may result in the production of cytokines and immunostimulation.

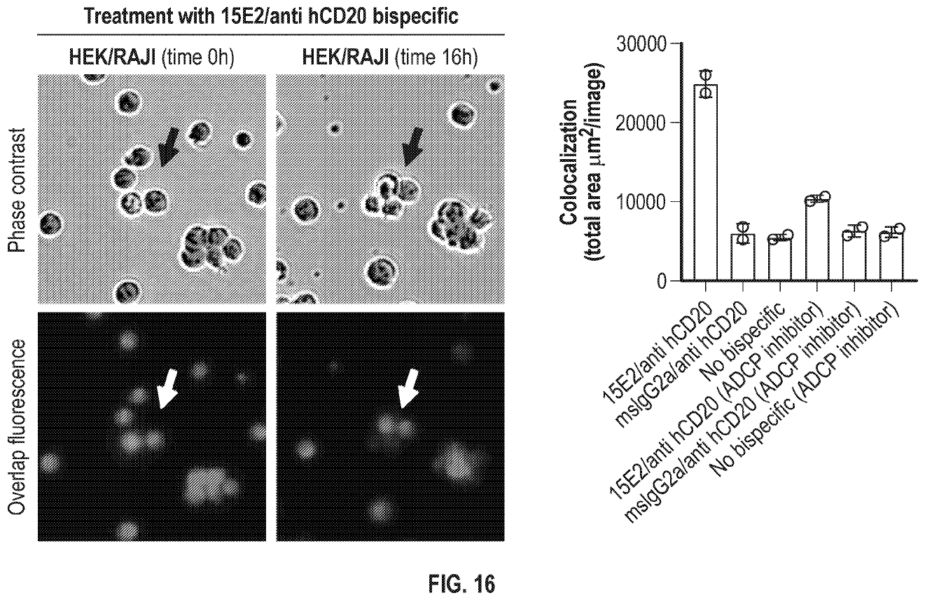

Previously, it was demonstrated that Dectin-1 expression in HEK 293 cells is necessary and sufficient to induce phagocytosis of various size beads coated with anti-Dectin-1 targeting antibody (see Example 1 and Example 2). To demonstrate phagocytosis of live target cells, a bispecific comprising an Dectin-1-targeting arm and a CD20-targeting arm was developed. In a co-culture assay of HEK 293 cells and CD20-expressing Raji cells, phagocytosis in cells treated with anti-Dectin-1/anti-hCD20 bispecific was observed, in contrast to isotype control bispecifics (FIG. 16). Furthermore, pre-incubation of cells with Latrunculin A, an inhibitor of phagocytosis that blocks actin polymerization, blocked phagocytosis of cells treated with anti-Dectin-1/anti-hCD20 bispecific. These findings indicate that Dectin-1 expression is sufficient to induce phagocytosis, and that co-targeting Dectin-1 and a target of interest with an Dectin-1-agonistic bispecific is sufficient to induce phagocytosis of a target cell.

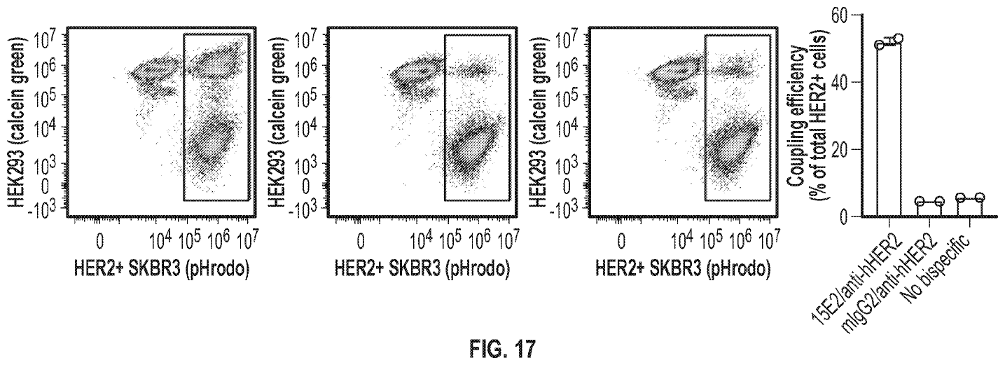

A proof-of-concept experiment was performed for co-targeting Dectin-1-expressing cells and HER2-positive breast cancer cells using an anti-Dectin-1/anti-HER2 bispecific antibody. Approximately 20% to 25% of invasive breast cancers exhibit overexpression of the human epidermal growth factor receptor HER2 tyrosine kinase receptor. As shown in FIG. 17, anti-Dectin-1 (15E2)/anti-HER2 bispecific induced coupling of Dectin-1- and HER2-expressing cells. This interaction is thought to promote synapse formation between effector and target cells, as Dectin-1 clustering induces cytokine secretion by effector cells, triggers phagocytosis of target cells, and leads to neo-antigen presentation and activation of adaptive immune cells (B and T cells).

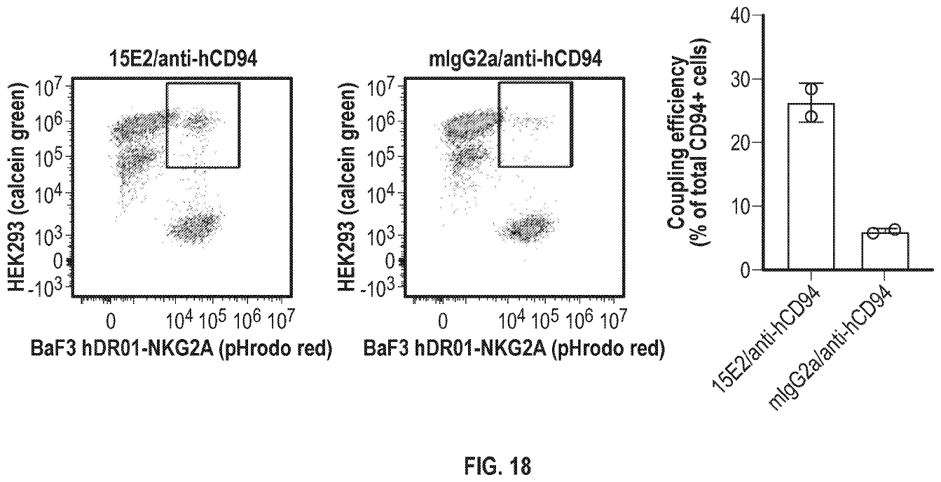

Finally, an anti-Dectin-1 (2M24)/anti-hCD94 bispecific was also evaluated. Large granular lymphocyte (LGL) leukemia is a rare chronic lymphoproliferative disease of T cell and natural killer (NK) cell lineage. CD94/NKG2 is a family of C-type lectin receptors which are expressed predominantly on the surface of NK cells and a subset of CD8+T-lymphocytes. As shown in FIG. 18, an anti-Dectin-1 (2M24)/anti-hCD94 bispecific induced coupling of Dectin-1-expressing cells and CD94-expressing cells. Thus, bispecific antibodies that bind Dectin-1 can mediate coupling of Dectin-1-expressing cells with a variety of target cells.

### Example 3: Generation of Bispecific Anti-Dectin-1 Antibodies Using Streptavidin-Biotin

This example describes the biochemical and functional characterization of bispecific antibodies that bind Dectin-1 generated using streptavidin-biotin conjugation.

### Materials and Methods

### Generation of Bispecifics

mSA was genetically fused to either Fab 2M24 or full length 2M24. Chimeric fusions were incubated with biotinylated target antibodies to generate a bispecific comprising a Dectin-1-binding arm and a second arm binding a target receptor or protein of interest.

Full-length 2M24 sequence fused to mSA:
(SEQ ID NO: 15)
QVQLVQSGAEVKKPGASVKVSCKSSGYTFTDYYIHWVRQAPGQGLEWMGW
INPNSGDTNYAQKFQGRITMTRDTSISTAYLELSRLRSDDTAVFYCARNS
GSYSFGYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSPGKGGGSG
GGSGGGSEFASAEAGITGTWYNQHGSTFTVTAGADGNLTGQYENRAQGTG
CQNSPYTLTGRYNGTKLEWRVEWNNSTENCHSRTEWRGQYQGGAEARINT
QWNLTYEGGSGPATEQGQDTFTKVKPSAASGS
Fab 2M24 sequence fused to mSA:
(SEQ ID NO: 17)
QVQLVQSGAEVKKPGASVKVSCKSSGYTFTDYYIHWVRQAPGQGLEWMGW
INPNSGDTNYAQKFQGRITMTRDTSISTAYLELSRLRSDDTAVFYCARNS
GSYSFGYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVGGGSGGGSGGGSEFASAEAGITGTWYNQHGSTFT
VTAGADGNLTGQYENRAQGTGCQNSPYTLTGRYNGTKLEWRVEWNNSTEN
CHSRTEWRGQYQGGAEARINTQWNLTYEGGSGPATEQGQDTFTKVKPSA
ASGSAAAGASHHHHHH

### Antibody-Dependent Targeted Phagocytosis of Phrodo-Labeled Beads

Antibody-dependent targeted phagocytosis of Phrodo-labeled beads was performed as described in Example 2. To monitor phagocytosis by flow cytometry, HEK cells overexpressing Dectin-1 were incubated with biotin beads conjugated to Fab 2M24-mSA for 30 minutes on ice or at 37° C. for 30 minutes, followed by washing with PBS twice. Phagocytosis was assessed by detecting activated Phrodo red within the HEK cell/beads duplet population by flow cytometry in the PE channel using a CytoFlex flow cytometer (Beckman Coulter, Atlanta, GA).

### Results

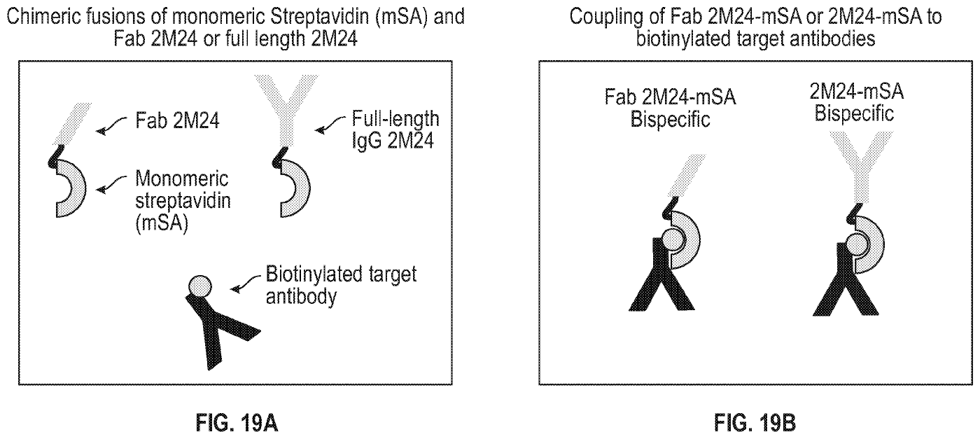

To enable the efficient generation of bispecific antibodies, a novel strategy was developed which utilizes the high affinity interaction of streptavidin and biotin. A monomeric streptavidin (mSA) construct was fused to the Fc-domain of 2M24, or CH1 domain of Fab 2M24. The recombinant fusion proteins were incubated with various biotinylated antibodies of interest to assemble the bispecifics. A schematic of this strategy is shown in FIGS. 19A-19B.

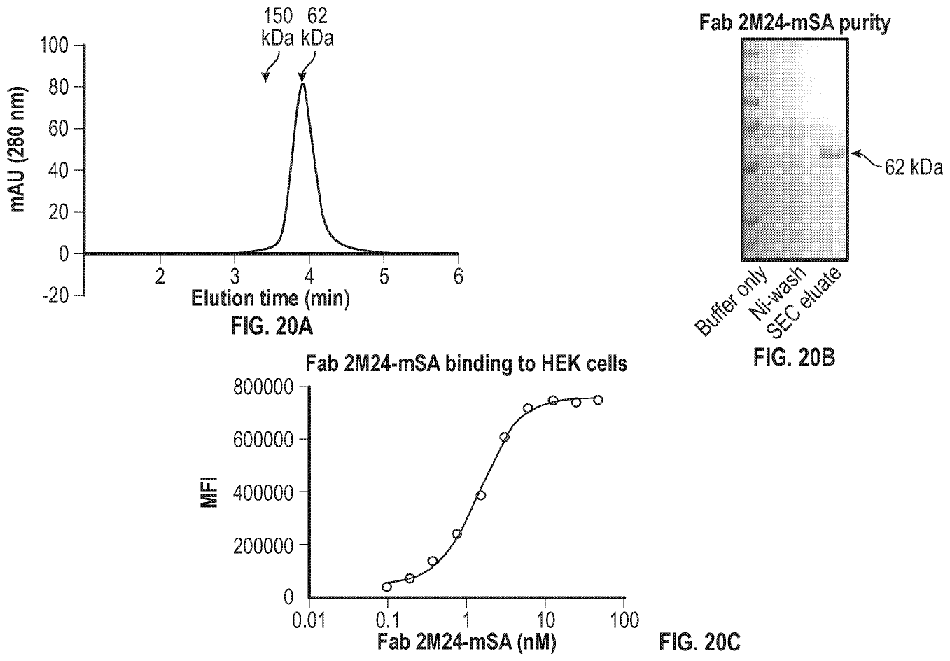

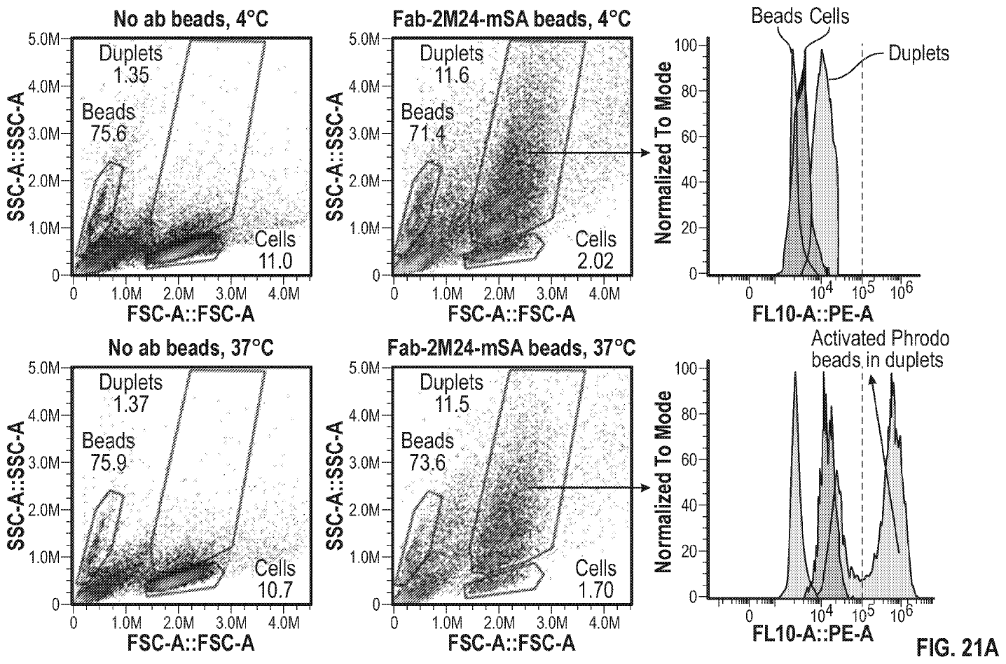

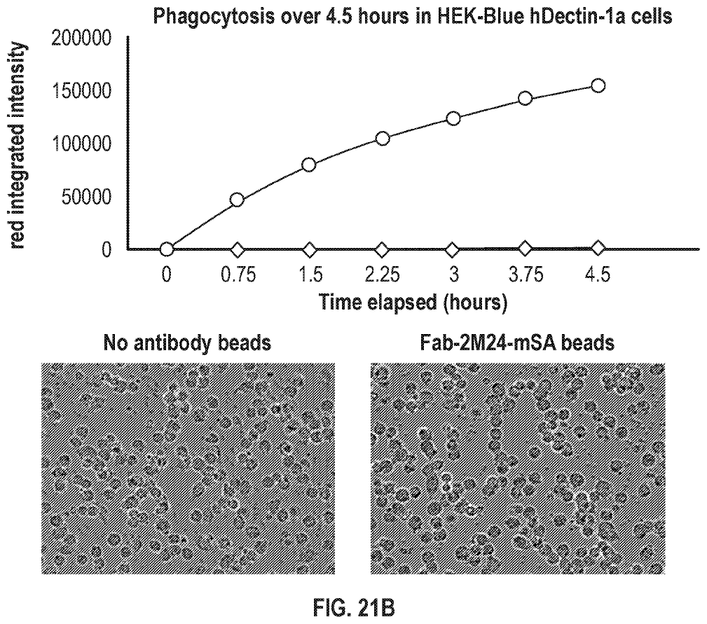

This fusion technology enables the high-throughput generation and screening of bispecific antibodies. To test this approach, a Fab 2M24-mSA fusion protein was generated and purified. As shown in FIGS. 20A-20C, the Fab 2M24-mSA fusion showed high affinity binding (EC50=1.45 nM) to Dectin-1 expressing cells. This Fab 2M24-mSA fusion protein can be combined with various biotinylated antibodies against targets of interests. Moreover, the Fab 2M24-mSA fusion also induced binding and phagocytosis of beads by Dectin-1-expressing HEK 293 cells (FIGS. 21A-21B), indicating that the Fab version of the 2M24 antibody can efficiently promote phagocytosis in cells expressing Dectin-1.

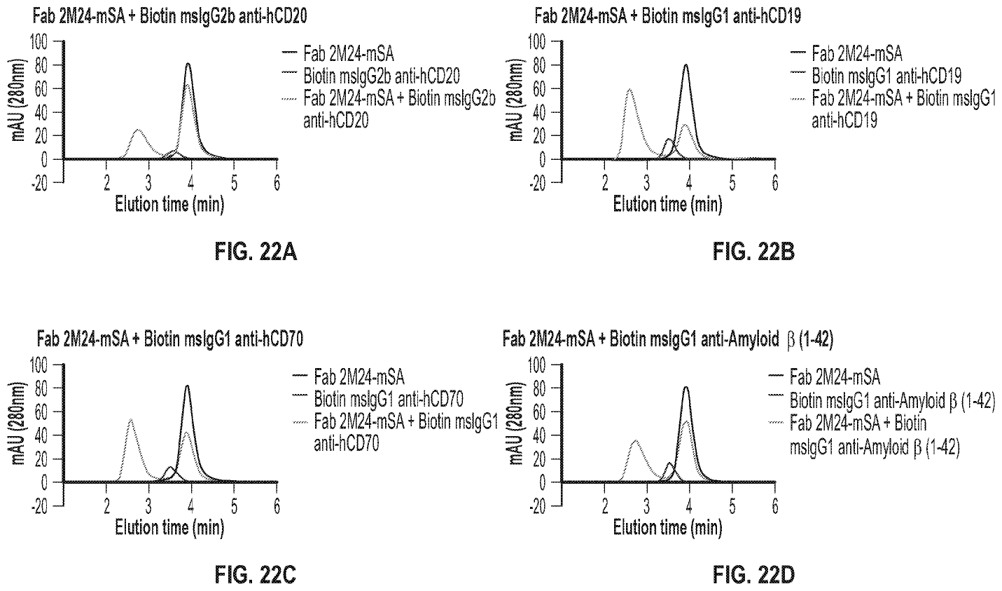

Using the anti-Dectin-1-streptavidin fusion, bispecifics against various targets (e.g., CD20, CD19, CD70, amyloid B (1-42)) were developed. As shown in FIGS. 22A-22D, these bispecifics showed high homogeneity based on HPLC analysis. These data demonstrate robust feasibility of this technology for bispecific antibody generation.

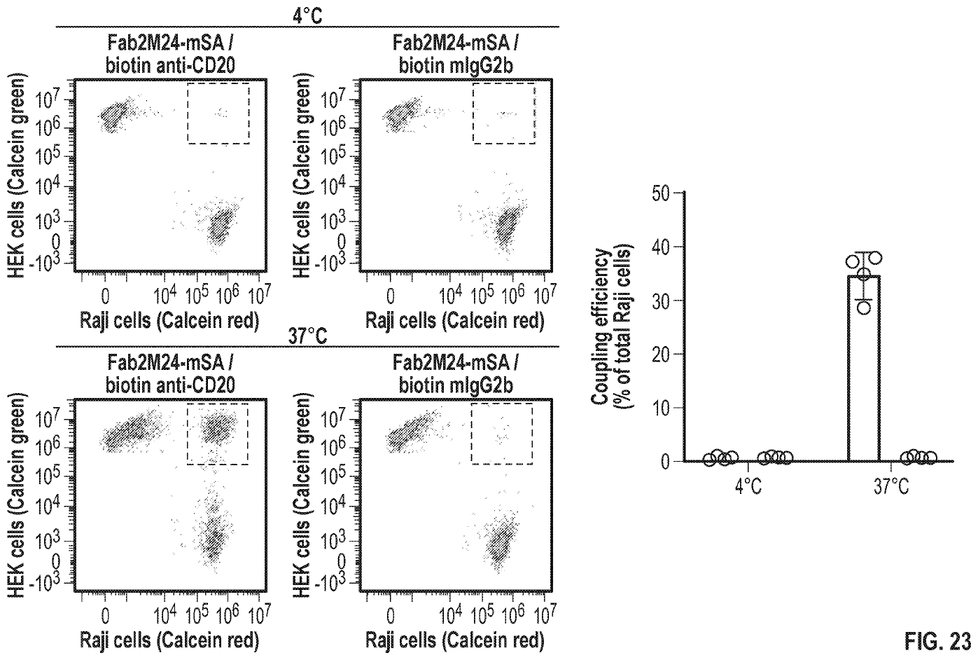

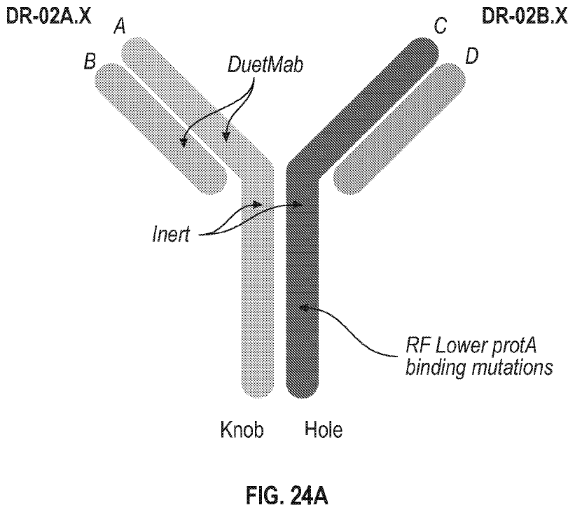

Next, the anti-Dectin-1 bispecifics generated using the Fab 2M24-mSA fusion protein were evaluated for their ability to induce cell coupling. As shown in FIG. 23, the Fab 2M24-mSA/biotin anti-hCD20 bispecific induced coupling of Dectin-1-expressing HEK293 cells and CD20-expressing B cells (Raji cell line). This interaction can promote Dectin-1 clustering, which induces cytokine secretion by effector cells, triggers phagocytosis of target cells, and leads to neo-antigen presentation and activation of adaptive immune cells (B and T-cells).

### Example 4: Bispecific Design for Development of a Human Bispecific Antibody Targeting Dectin-1 and a Disease Target or Antigen

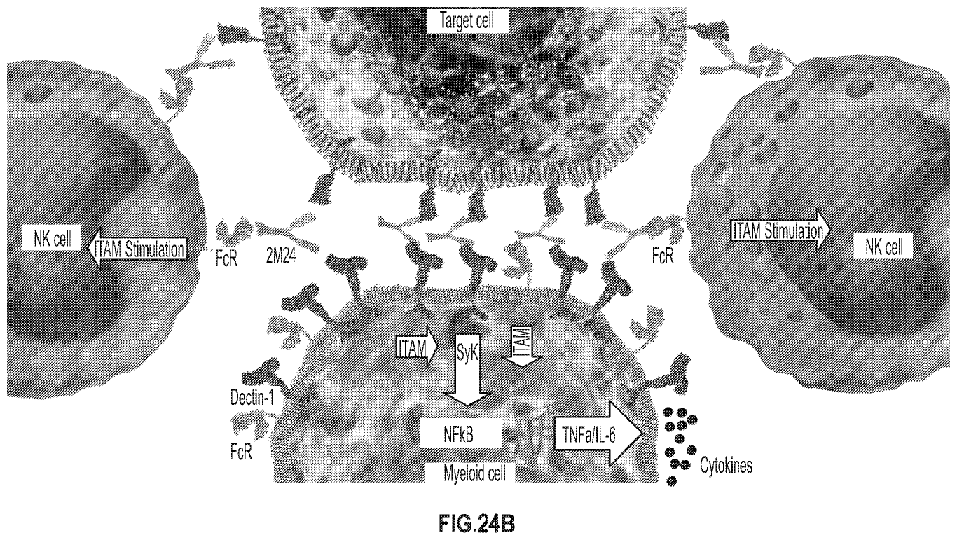

To enable the assembly and efficient production of highly purified and active bispecifics, design principles were adopted based on previously reported strategies including “knobs-into-holes” (Ridgway, 1996; U.S. Pat. No. 8,679,785B2), DuetMab (Mazor, 2015; patent EP3452089A2), single-step Protein A and G avidity purification methods (Ollier, 2019; AU2018204314B2), and mutations to eliminate FcR binding (patent WO 2016/081746 A2). Assembly of complete bispecific involves expression of 4 individual subunits, e.g., cloned into expression vectors such as pFUSE. A diagram of an exemplary anti-Dectin-1 bispecific antibody is shown in FIG. 24A.

As shown in Table 1, bispecific antibodies using this design were constructed for proof-of-concept studies. These bispecific antibodies have one arm that targets hDectin-1 and a second arm that targets hCD20. The bispecific antibodies described in Table 1 were generated by expressing all 4 chains and purifying to 95% purity and homogeneity. All bispecifics were found to bind their respective targets.

TABLE 1
Bispecific antibodies targeting human Dectin-1 and antigen
expressed on cancer cells/disease targets.

| Name | Target 1 | Target 2 |
|---|---|---|
| 2M24/CD20 | hDectin-1 (variable | hCD20 (variable domain |
| hIgG1 | domain from clone | based on Rituximab |
| (fucosylated | 2M24) | antibody) |
| or | | |
| afucosylated) | | |

Variable domains for the antibody arm opposite anti-Dectin-1 in Table 1 were as follows.

CD20 VH:
(SEQ ID NO: 24)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSA
CD20 VL:
(SEQ ID NO: 25)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIK

These hDectin-1 bispecific antibodies engage 3 targets: Dectin-1 on myeloid cells, CD20 on a target cell or disease-causing agent, and Fc receptors on myeloid and NK cells, eliciting robust immune stimulation and phagocytosis (FIG. 24B). In particular, bispecific antibodies with a non-fucosylated, active hIgG1 Fc domain allow the bispecific antibody to recruit myeloid cells (e.g., monocytes, macrophages, and dendritic cells) and natural killer (NK) cells to eliminate disease-causing target cells, such as tumor cells expressing specific antigens. In the context of cancer, and without wishing to be bound to theory, dual engagement of Dectin-1 and Fcγ receptors on myeloid and NK cells is thought to elicit a strong immune response that ultimately eliminates cancer cells via the following actions: (1) bispecific antibody-induced cross-linking of Dectin-1 and Fcγ receptors leads to ITAM-dependent activation of downstream inflammatory pathways and release of immuno-modulatory cytokines and cytotoxic proteins (proteases, perforin) which modulates the tumor microenvironment and may directly kill targeted cells; (2) bispecific antibody-induced clustering of Dectin-1 and Fcγ receptors triggers phagocytosis and elimination of targeted cancer cells by monocytes, macrophages and dendritic cells; and (3) phagocytosed antigens are presented by macrophages and DCs, a process that triggers a T cell immune response aimed at eliminating cancer cells.

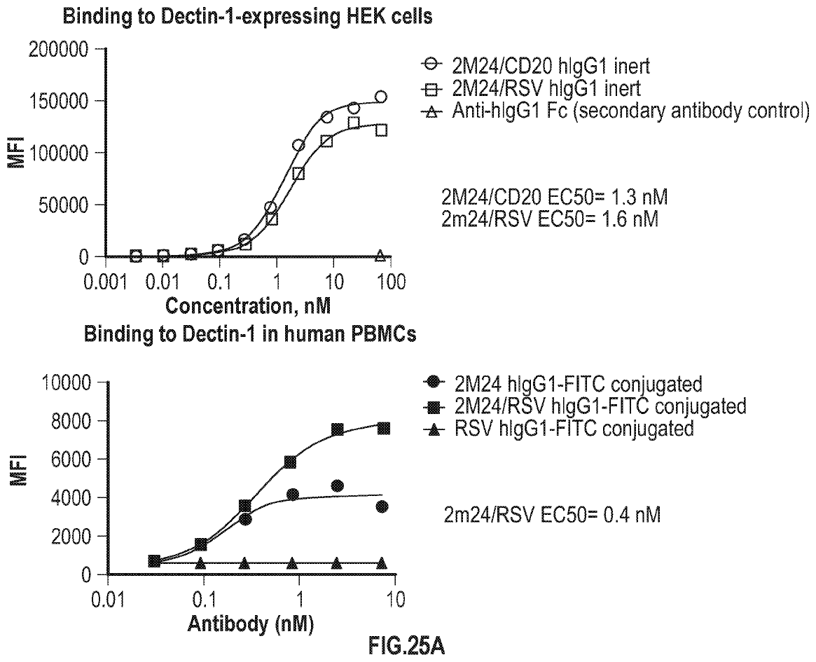

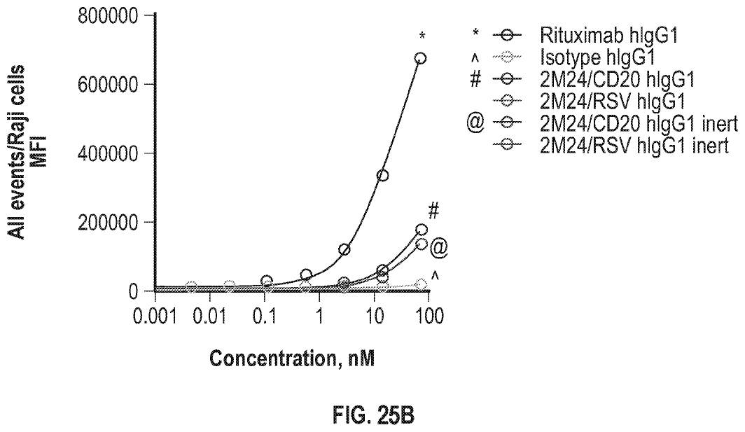

The 2M24/CD20 bispecific antibody described in Table 1 was tested for binding to cells expressing human Dectin-1 or CD20. 2M24/RSV was used in all assays as an isotype control for the target binding arm. The bispecific variants tested here contained mutations in the hIgG1 Fc domain (hIgG1 inert) that eliminate Fc binding to Fc receptors (L234A, L235E, and G237A, according to EU numbering). Binding of 2M24/CD20 or 2M24/RSV bispecific to HEK293 cells stably expressing human Dectin-1 was assessed by flow cytometry (FIG. 25A). 2M24/CD20 hIgG1 inert bispecific antibodies were able to bind cells expressing human Dectin-1 with similar affinities (cell-based binding EC50 values of 1.4 and 1.7 nM, respectively). Thus, 2M24/CD20 bispecific antibodies displayed high affinity binding to Dectin-1-expressing HEK293 cells.

Binding of Rituximab, 2M24/CD20, or 2M24/RSV hIgG1 active or inert bispecific antibodies was also assessed using the CD20-expressing B cell lymphoma Raji cell line (FIG. 25B). 2M24/CD20 bispecific (active or inert hIgG1 isotypes) antibodies were able to bind to CD20-expressing Raji cells, but with at least 10-fold reduced affinity compared to Rituximab. Without wishing to be bound to theory, it is thought that the difference in CD20 binding affinity between 2M24/CD20 bispecific and Rituximab is likely mediated by the loss of avidity (monovalent versus bivalent binding) in the bispecific antibody.

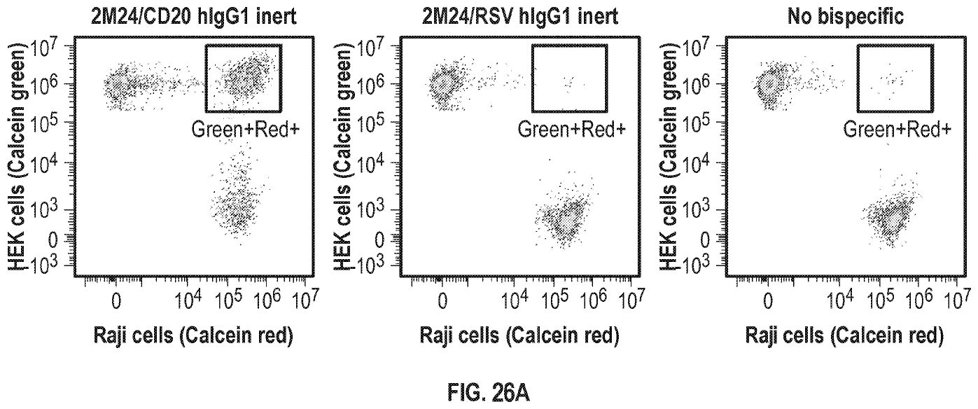

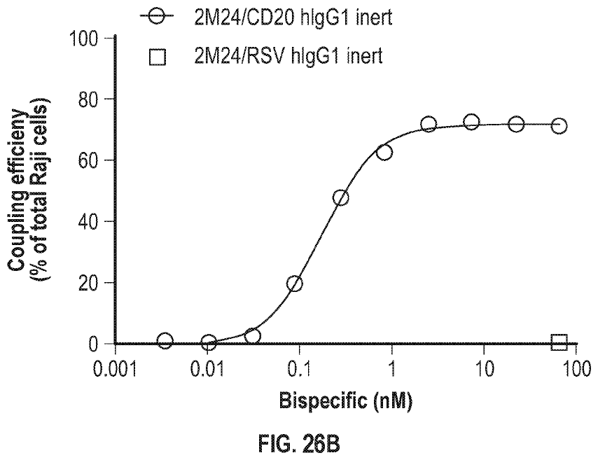

Next, the ability of 2M24/CD20 bispecific antibody to induce coupling of cells expressing hDectin-1 and cells expressing hCD20 was assayed. Dectin-1-expressing HEK293 cells (effector) and CD20-expressing Raji cells (target) were differentially labeled with calcein green (effector) or calcein red (target) dyes. Labeled cells were co-cultured and treated with hIgG1 inert 2M24/CD20 or 2M24/RSV (control) bispecific antibody to induce effector:target coupling. Successful coupling of effector:target cells was indicated by the double-positive staining (Calcein green+, calcein red+, square box; FIG. 26A). Coupling efficiency (quantified as the percentage of total target cells that binds or couples to effector cells) was assayed using dose titration of bispecific antibody in co-cultures of effector:target cells (FIG. 26B).

These results indicate that 2M24/CD20 bispecific antibody can couple Dectin-1-expressing ‘effector’ cells and CD20-expressing ‘target’ cells with a potent EC50 of 0.17 nM. Despite the low affinity binding of 2M24/CD20 bispecific to CD20 on Raji cells (FIG. 25B), the 2M24/CD20 was highly efficient at coupling. These findings suggest that 2M24/CD20 binding affinity is enhanced by the high expression of Dectin-1 or CD20 on both effector and target cells (avidity), thereby promoting efficient coupling of the two cells. Based on these findings, it is thought that 2M24/CD20 bispecific antibody could effectively engage Dectin-1-expressing monocytes, macrophages or dendritic cells with target disease cells, such as B cell lymphoma, which express high levels of CD20. Effector: target engagement is the first step in the MOA of 2M24 bispecific antibodies.

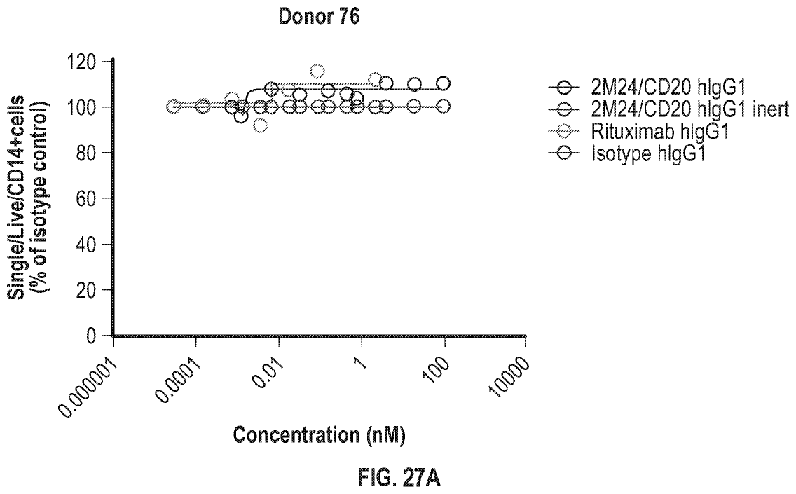

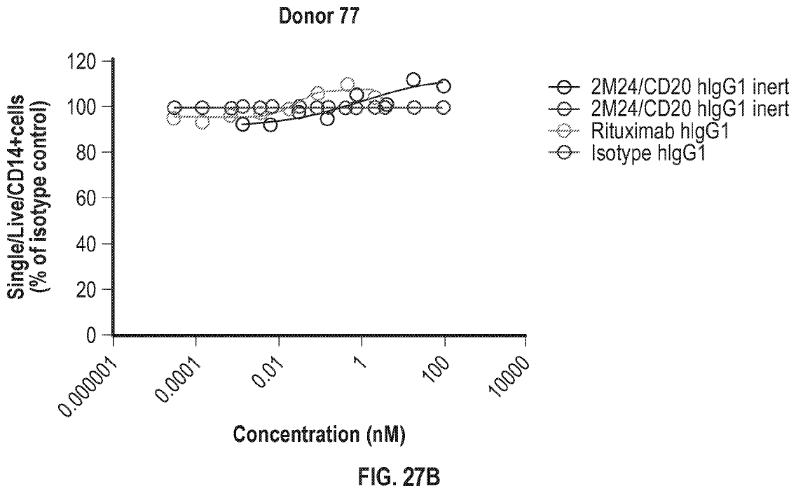

Human IgG1 active isotype binds Fcγ receptors on NK cells or monocytes. Therefore, it was assessed whether the hIgG1 active isotype of 2M24/CD20 can trigger monocyte killing by NK cells (via antibody dependent-cellular cytotoxicity, ADCC) or other monocytes (Fratricide or antibody-dependent cellular phagocytosis, ADCP). In this scenario, the active hIgG1 domain of 2M24/CD20 engages the Fcγ receptors on NK cells or monocytes, and Dectin-1 receptor on monocytes, thereby inducing Fcγ-mediated activation and depletion of target. PBMCs from two healthy donors-donor 76 (FIG. 27A) and donor 77 (FIG. 27B) were treated with increasing concentrations of 2M24/CD20 bispecifics (hIgG1 active or inert isotypes) and rituximab for 24 h, and subsequently analyzed by flow cytometry to quantify the levels of live, CD14+ monocytes remaining (as a % of isotype controls). No decrease in the number of monocytes was found in either donor, indicating that 2M24/CD20 active IgG1 did not induce monocyte depletion. Without wishing to be bound to theory, it is thought that 2M24/CD20 hIgG1 (active isotype) should not affect the levels of monocytes and thus poses minimal risk of infection.

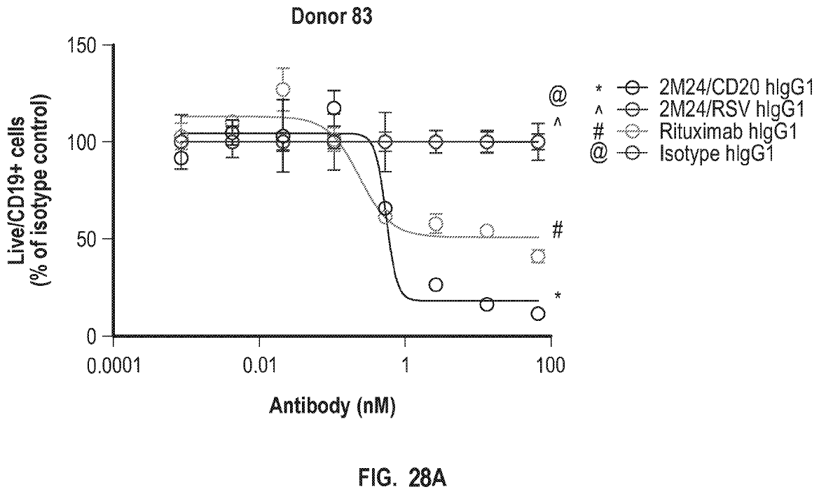

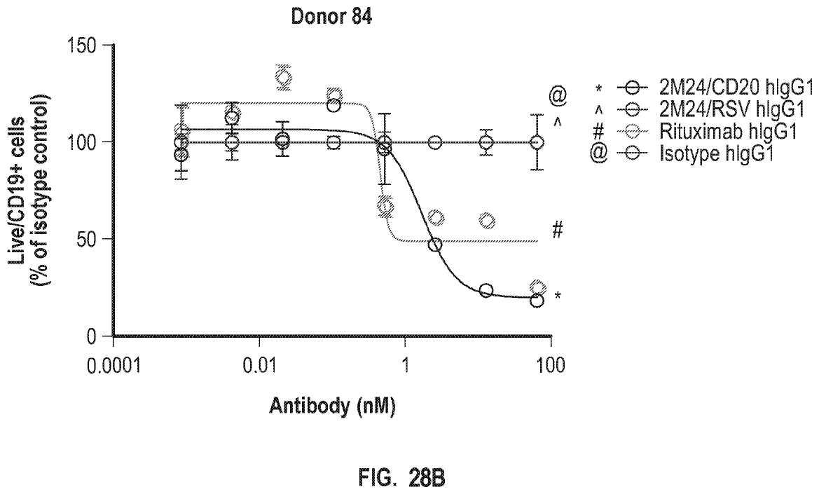

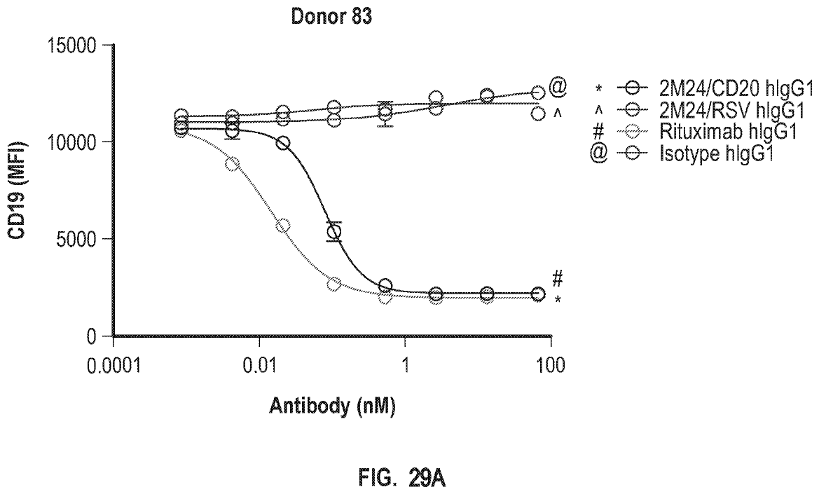

Based on the proposed MOA of 2M24/CD20 bispecific antibody (described in FIG. 24B), B cell depletion by 2M24/CD20 hIgG1 (active isotype) bispecific antibody or Rituximab was assessed in order to compare B cell depletion. PBMCs from two healthy donors—donors 83 (FIG. 28A) and 84 (FIG. 28B)—were treated with increasing concentrations of the indicated antibodies for 24 hours, and subsequently analyzed by flow cytometry to quantify the levels of remaining live, CD19+ B cells (reported as a % of B cells in isotype control-treated PBMCs). Thus, in two healthy donors, high concentrations of 2M24/CD20 bispecific antibody induced superior B cell depletion (˜80% reduction) compared to Rituximab (˜40% reduction), despite the bivalent binding nature of Rituximab and ˜10-fold difference in binding affinity (as shown in FIG. 25B). The unique mechanism of action of 2M24/CD20 active IgG1, which involves binding to Dectin-1 on myeloid cells, Fcγ receptors on NK cells and monocytes, and CD20 on target B cells (FIG. 24B), leads to an overall superior depletion of B cells compared to Rituximab. These data support the concept that Dectin-1 induced immune stimulation via 2M24/CD20 bispecific enhances depletion of the target cells.

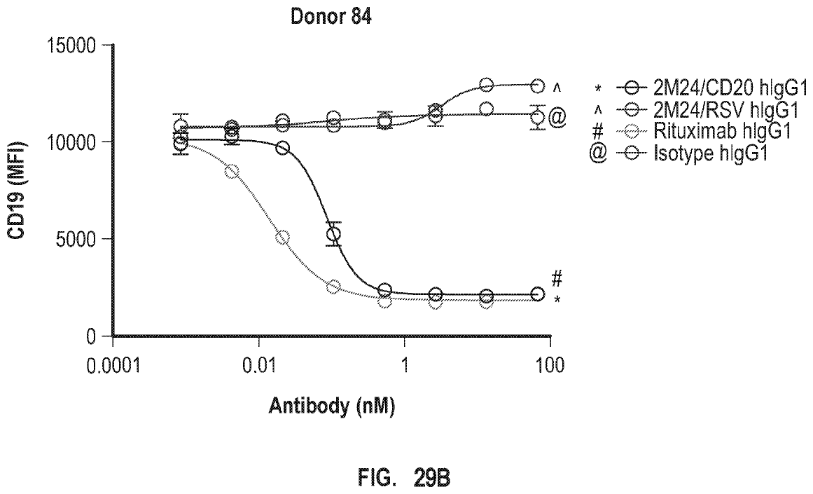

Ability of 2M24/CD20 hIgG1 (active isotype) bispecific antibody or Rituximab (hIgG1) to downregulate CD19 expression on B cells in a process known as shaving or trogocytosis was assessed. Expression of CD19+ on B cells from two healthy donors—donor 83 (FIG. 29A) and donor 84 (FIG. 29B)—was quantified by flow cytometry following a 24-hour incubation with increasing concentration of 2M24/CD20 hIgG1 (active isotype) bispecific antibody, Rituximab, or isotype controls. The mean fluorescent intensity (MFI) for CD19 staining using anti-CD19 (BV605 conjugated) was used to evaluate the effect of 2M24/CD20 bispecific and Rituximab on CD19 expression on B cells. In PBMCs from donor 83, the EC50 with respect to CD19 expression was 0.014 nM for rituximab and 0.080 nM for 2M24/CD20 hIgG1 bispecific (FIG. 29A). In PBMCs from donor 84, the EC50 with respect to CD19 expression was 0.013 nM for rituximab and 0.090 nM for 2M24/CD20 hIgG1 bispecific (FIG. 29B). Both 2M24/CD20 active IgG1 bispecific Ab and Rituximab led to downregulation of CD19 expression on B cells. Interestingly, Rituximab demonstrated at least five-fold more potent shaving compared to 2M24/CD20 bispecific Ab. Downregulation of target CD20 on B cells was previously reported as a mechanism by which malignant B cells escape Rituximab-mediated depletion (Beum, P. V. et al. (2006) J. Immunol. 176:2600-2609). These findings therefore suggest that 2M24/CD20 active IgG1 bispecific may be superior in depleting B-cells as compared to Rituximab due to reduced shaving potential.

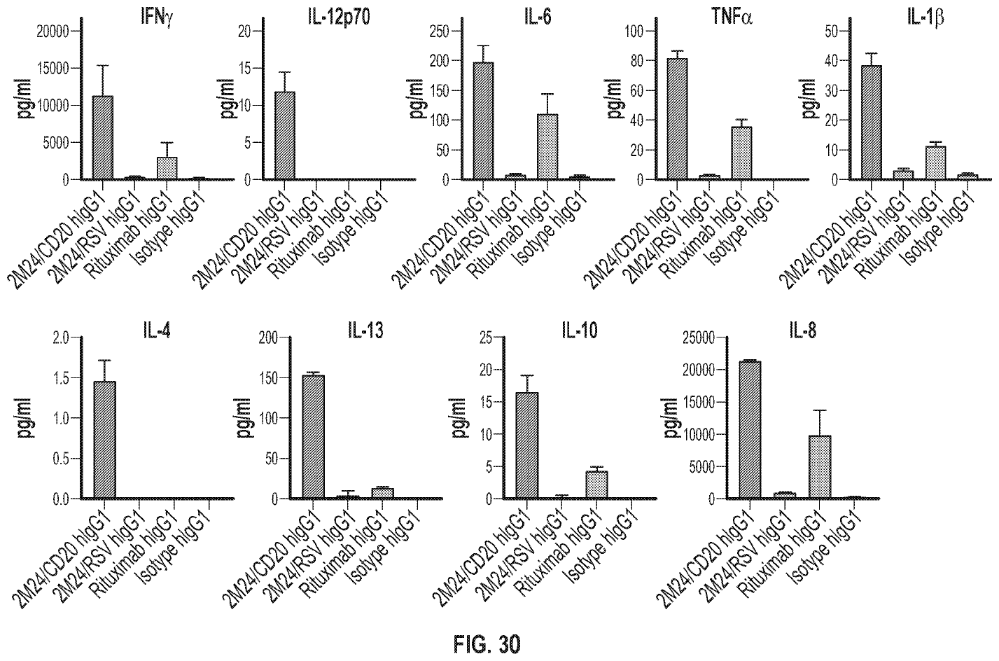

Immune stimulation triggered by 2M24/CD20 active IgG1 bispecific antibody led to secretion of a unique repertoire of cytokines compared to Rituximab (FIG. 30). ELISA-based (mesoscale discovery) quantification of cytokines was undertaken in supernatants isolated from healthy donor PBMCs treated with 2M24/CD20 active hIgG1 bispecific, Rituximab, or isotype controls. PBMCs were stimulated with antibodies overnight, and supernatants were subsequently analyzed by MSD. Cytokines tested were IFNγ, IL-12p70, IL-6, TNFα, IL-1β, IL-4, IL-13, IL-10, and IL-8. The results indicated that 2M24/CD20 active IgG1 triggered higher level and distinct cytokine activation in PBMCs compared to Rituximab. Furthermore, engagement of Dectin-1 and Fc receptor alone by the 2M24/RSV bispecific does not induce cytokine release, excluding the possibility of systemic cytokine activation. These findings highlight a unique MOA that distinguishes 2M24/CD20 active IgG1 bispecific antibody from Rituximab. Without wishing to be bound to theory, these findings further indicate that 2M24/CD20 could trigger release of Th1 and Th2-type of responses and promote immune stimulation of tumor microenvironment.

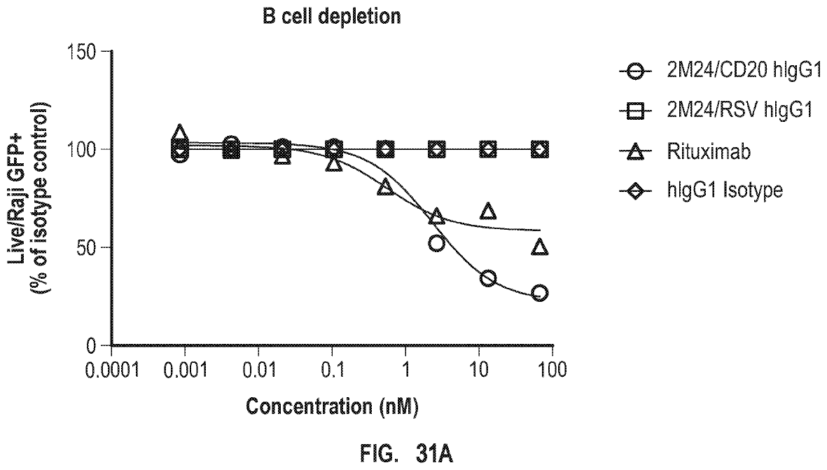

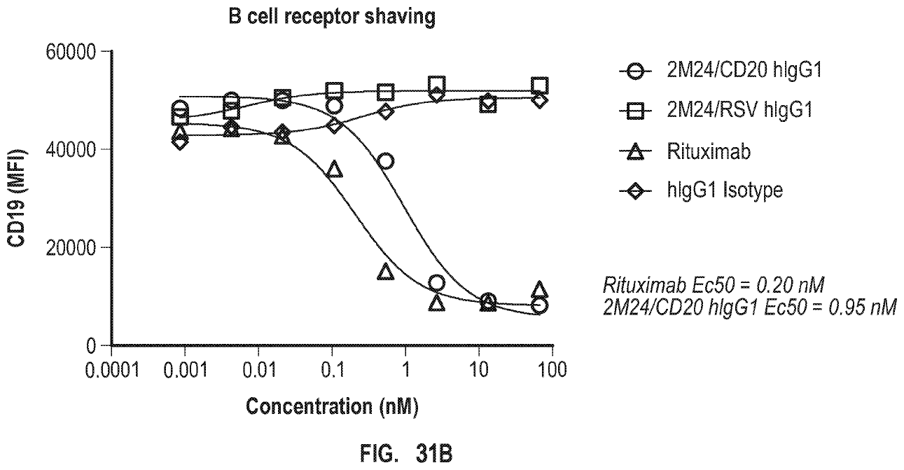

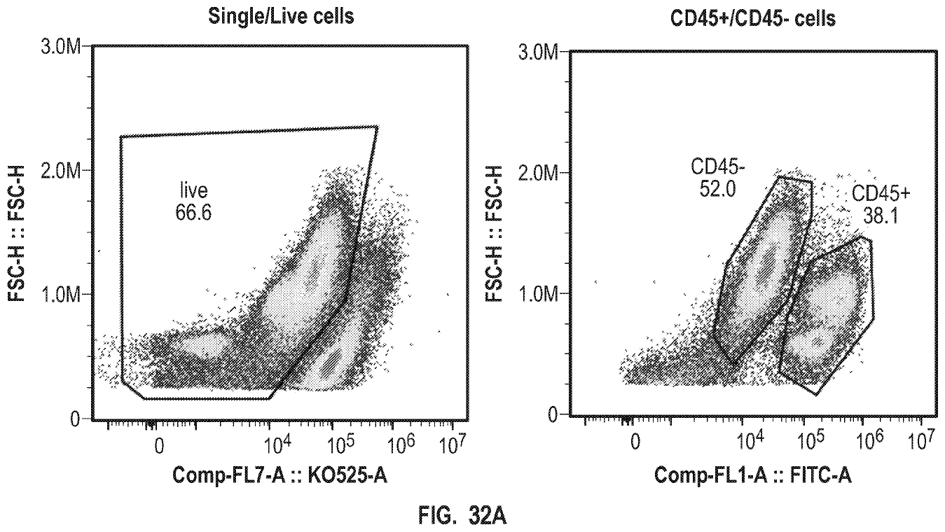

2M24/CD20 hIgG1 (active isotype) bispecific antibody was also found to induce superior B-cell depletion and lower CD19 shaving compared to Rituximab in co-cultures of human macrophages and GFP-expressing Raji B cells. Co-cultures of human macrophages and Raji-GFP cells (3:1 ratio) were analyzed by flow cytometry in the presence of 2M24/CD20 hIgG1 (active isotype) bispecific, 2M24/RSV control, fucosylated Rituximab or isotype hIgG1 control (FIG. 31A). Co-cultures were incubated at 37° C. for 24 hours and then stained with a PE a-CD206 Ab to label macrophages and a BV-605 a-CD19 antibody to label Raji cells. The number of the remaining live/Raji-GFP+ cells was assessed in the end of the experiment. The primary antibodies were used in a serial dose titration. CD19 was assessed on Raji-GFP cells after 24 hours (FIG. 31B), with B-cell receptor shown as the reduction in the CD19 MFI in the presence of a-Dectin-1/a-hCD20 bispecific or Rituximab. The EC50 with respect to CD19 expression was 0.020 nM for rituximab and 0.95 nM for 2M24/CD20 hIgG1 bispecific. These results demonstrate enhanced B-cell depletion (Fcγ receptor mediated) by the 2M24/CD20 bispecific antibody compared to Rituximab. Rituximab reduced the B-cell receptor CD19 surface levels more potently than the a-Dectin-1/a-hCD20 bispecific antibody. Similarly, B-cell receptor shaving has been observed for CD20 by Rituximab, and the reduction of the CD20 limits the effectiveness of Rituximab to deplete B-cell. Without wishing to be bound to theory, it is thought that these data indicate that the superiority of the 2M24/CD20 hIgG1 (active isotype) bispecific to deplete B-cells is due to the lower B-cell receptor shaving compared to Rituximab. This highlights a differential mechanism of cell depletion by 2M24/CD20 hIgG1 (active isotype) bispecific.

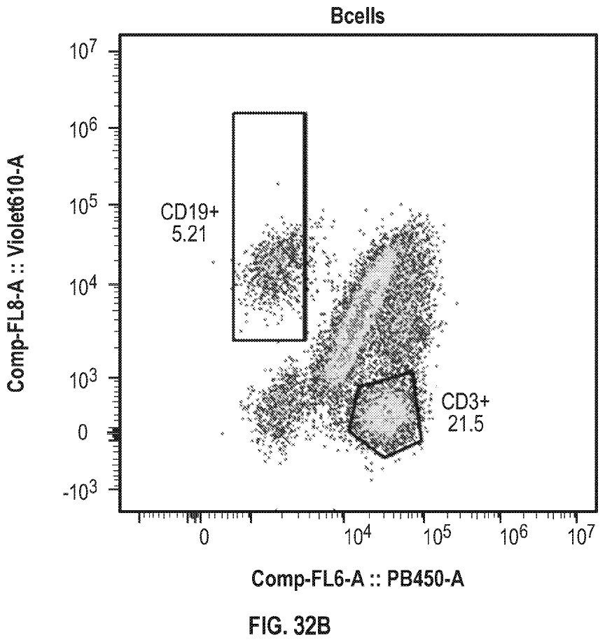

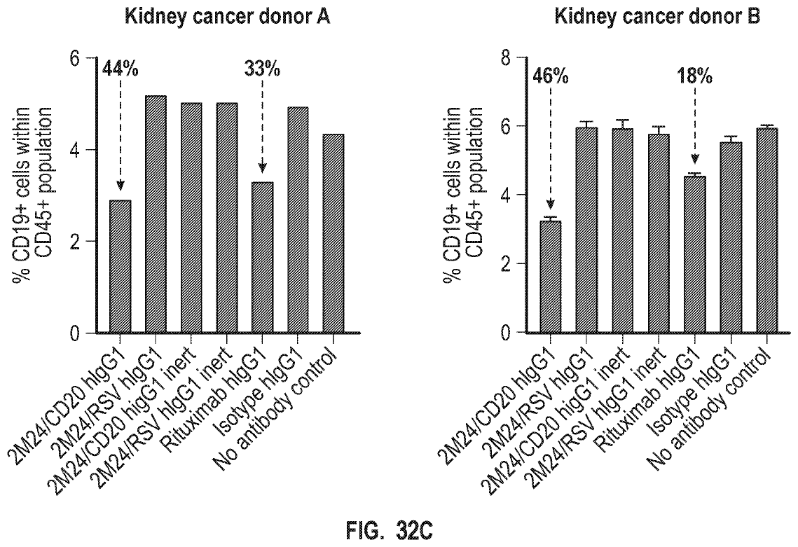

B-cell depletion was also analyzed in single cell suspensions from kidney cancer tissue biopsies. Single cell suspensions from two Kidney cancer tissue biopsies were analyzed by flow cytometry in the presence of 2M24/CD20 hIgG1 (active or inert) bispecific antibody, 2M24/RSV hIgG1 controls, fucosylated Rituximab, and respective isotype controls. Kidney cancer tissue biopsies were dissociated to single cell suspensions and treated with primary antibodies (2 μg/ml) for 24 hours at 37° C. Immune cell populations were analyzed by flow cytometry (FIGS. 32A & 32B). The number of the remaining live B cells was assessed by an anti-CD19 antibody and expressed as percentage of the CD45+ immune cell population (FIG. 32C). 2M24/CD20 active IgG1 bispecific antibody induced superior tissue B cell depletion as compared to Rituximab in single cell suspension of kidney cancer biopsies. The 2M24/CD20 hIgG1 (active isotype) bispecific antibody reduced B cells in the two kidney cancer donor biopsies by 44% and 46% (respectively), whereas Rituximab induced a B cell reduction of 33% and 18%, respectively (FIG. 32C). The data support the functionality of the 2M24/CD20 hIgG1 (active isotype) bispecific to deplete cells in cancer tissues via Dectin-1 induced immune stimulation and Fcγ receptor engagement. Without wishing to be bound to theory, it is thought that, since Dectin-1 is predominantly expressed on tumor associated macrophages (TAMs) in the above-described biopsies, 2M24/CD20 hIgG1 (active isotype) bispecific antibody may engage TAMs to enhance the target cell depletion.

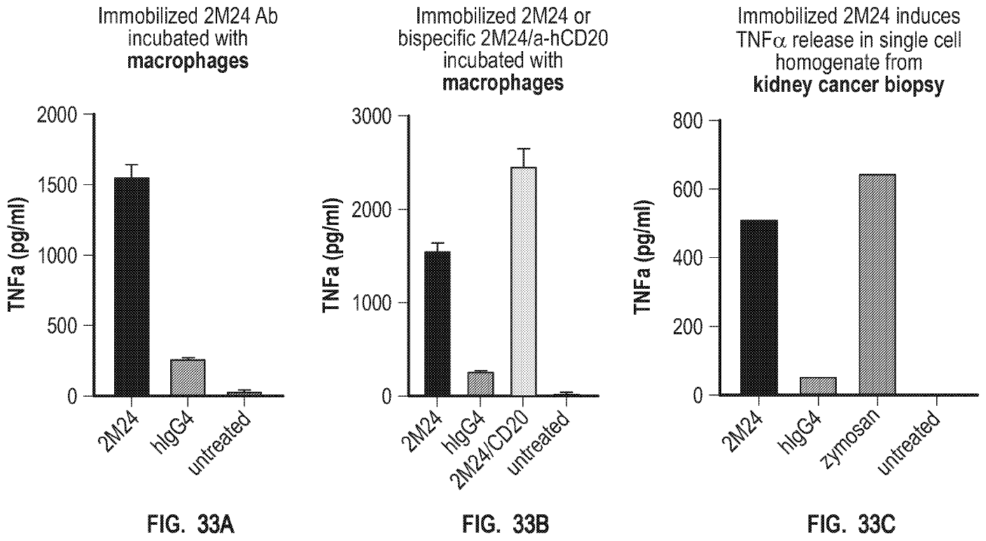

Cytokine secretion by cultured macrophages and single cell suspension of kidney cancer biopsies stimulated with immobilized anti-Dectin-1 antibody (clone 2M24) or 2M24/CD20 bispecific antibody was tested. The anti-Dectin-1 antibody (clone 2M24), isotype control or the 2M24/CD20 bispecific antibody were immobilized overnight in U-bottomed polypropylene microtiter plates at 10 ug per well, followed by culture of human monocyte-derived macrophages (FIGS. 33A & 33B) or single cell suspension from kidney cancer biopsy (FIG. 33C). The cells were cultured for 24 hours and evaluation of TNFα secretion in the supernatant was assessed by ELISA. As a positive control, cells were stimulated with zymosan. Anti-Dectin 1 antibody (clone 2M24) was found to induce Dectin 1-clustering and TNFα secretion from human macrophages. These data provide evidence that the parental anti-Dectin-1 antibody (clone 2M24) can promote immune-stimulation in primary macrophage cultures as well as in single cell homogenate of cancer biopsies. Since Dectin-1 is expressed in myeloid cells, tumor associated macrophages in the cancer biopsies are expected to produce cytokines in response to the anti-Dectin-1 antibody stimulation. This promotes the transition of the tumor associated macrophages from anti-inflammatory to pro-inflammatory with strong anti-tumoral effects. Moreover, monovalent binding of the 2M24/CD20 bispecific antibody to Dectin-1 was sufficient to promote Dectin-1 clustering and immune-stimulation on macrophages.

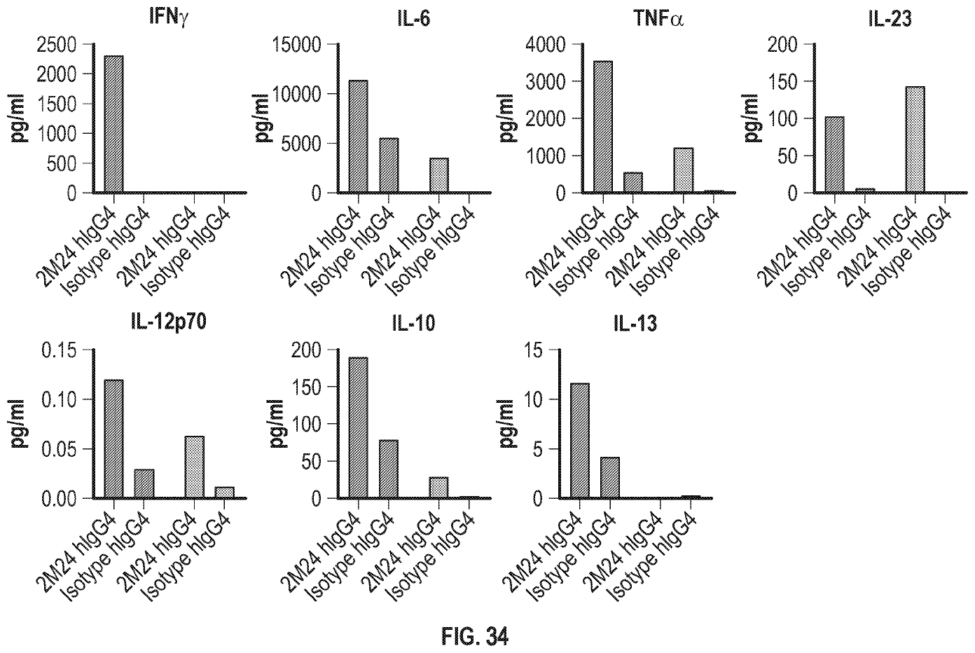

Immune stimulation by immobilized anti-Dectin-1 antibody in single cell suspensions from kidney cancer biopsies were also analyzed (FIG. 34). Single-cell suspensions from kidney cancer biopsies were treated with immobilized anti-Dectin-1 antibody (clone 2M24) or isotype control hIgG4 antibody for 24 h. Supernatants were analyzed by ELISA for the release of various cytokines, including IFNγ, IL-6, TNFα, IL-23, IL-12p70, IL-10, and IL-13. These results show that activation of Dectin-1 on myeloid cells (in this example, Dectin-1 is expressed predominantly by tumor-associated macrophages, TAMs), elicited the release of specific repertoire of cytokines that are either directly downstream of Dectin-1 signaling pathway, or indirectly through activation of other immune cells. Without wishing to be bound to theory, it is thought that Dectin-1 engagement by 2M24 bispecific antibody promotes immune stimulation that could modulate the tumor microenvironment to support the elimination of target-expressing cancer cells.

### Example 5: Characterization of a Bispecific Antibody Targeting Dectin-1 and CD20

This Example describes the further characterization of a bispecific antibody targeting human Dectin-1 and human CD20. The anti-Dectin-1 arm included the variable domains of 2M24, and the anti-CD20 arm included the variable domains of Rituximab (see SEQ ID Nos: 24 and 25 for VH and VL domains, respectively).

### Materials and Methods

### CD16 Expression on NK Cells

Human PBMCs from a healthy donor were treated with a serial dilution of 2M24/CD20 hIgG1 KIF, Rituximab KIF, and isotype control RSV hIgG1 KIF antibodies. After 24 hours of treatment, PBMCs were stained with antibodies against lineage-specific markers for flow cytometry analysis. CD16 expression on CD56+ NK cells was quantified and compared to expression levels in the isotype control treated group.

### CD19 Expression on B Cells

Human PBMCs from a healthy donor were treated with 0.1 nM of 2M24/CD20 hIgG1 KIF, Rituximab KIF, and isotype control RSV hIgG1 KIF antibodies. After 24 hours of treatment, PBMCs were stained with antibodies against lineage-specific markers for flow cytometry analysis. CD19 expression (MFI) on B cells was quantified.

### B Cell Depletion in PBMCs

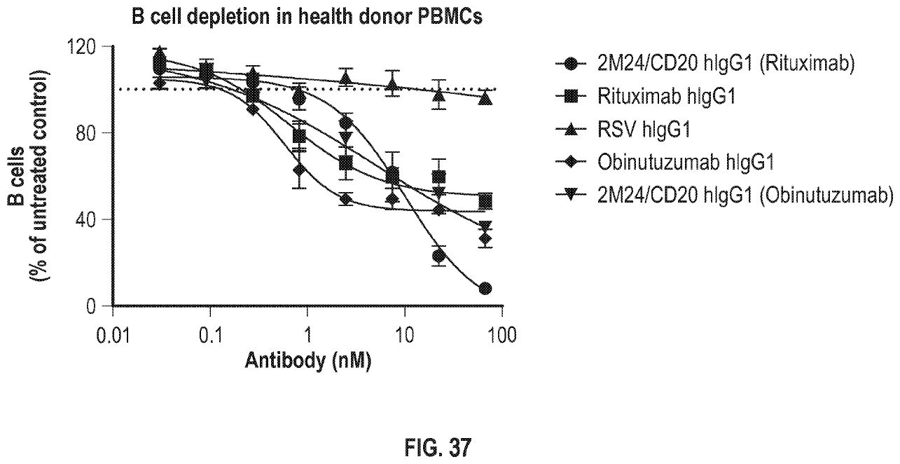

Human PBMCs from a healthy donor were treated with a serial dilution of the indicated antibodies. After 24 hours of treatment, PBMCs were stained with antibodies against lineage-specific markers for flow cytometry analysis. B cells were quantified relative to an untreated control group (indicated by the dotted line in FIG. 37).

### B Cell Depletion in Kidney Cancer Biopsies

Single-cell suspension was generated from kidney cancer biopsy and the cells were treated with 2M24/CD20 hIgG1, 2M24/RSV hIgG1, Rituximab hIgG1, and isotype control RSV hIgG1 antibodies. After 24 hours of treatment, the cells were stained with antibodies against lineage-specific markers for flow cytometry analysis. B cells were quantified as the percentage of CD19+ cells within the CD45+ immune cell population.

### Results

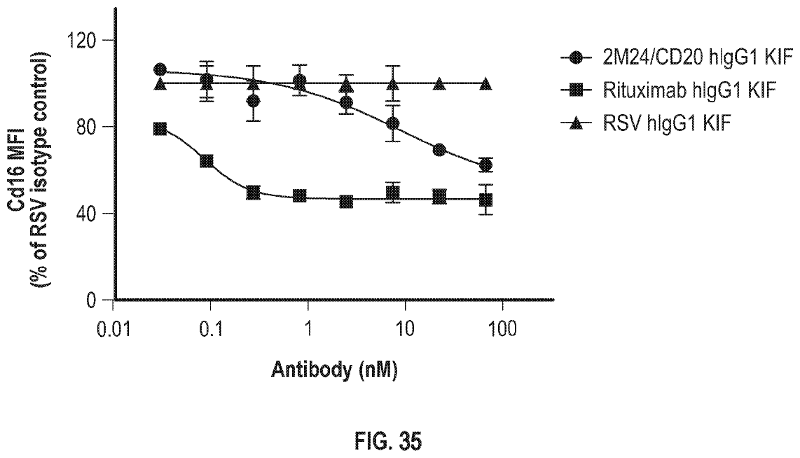

First, the effect of the 2M24/CD20 bispecific on CD16 expression was examined in human NK cells. CD16 is required for ADCC activity by NK cells, therefore the loss of CD16 expression can decrease the cytotoxic potential of NK cells. Rituximab induced potent and robust shedding of CD16 on NK cells compared to 2M24/CD20 hIgG1 KIF (FIG. 35). In contrast, CD16 levels on NK cells were better maintained after 2M24/CD20 bispecific antibody treatment compared to rituximab treatment. Without wishing to be bound to theory, it is thought that 2M24/CD20 bispecific has the potential to better preserve NK cell cytotoxic potential.

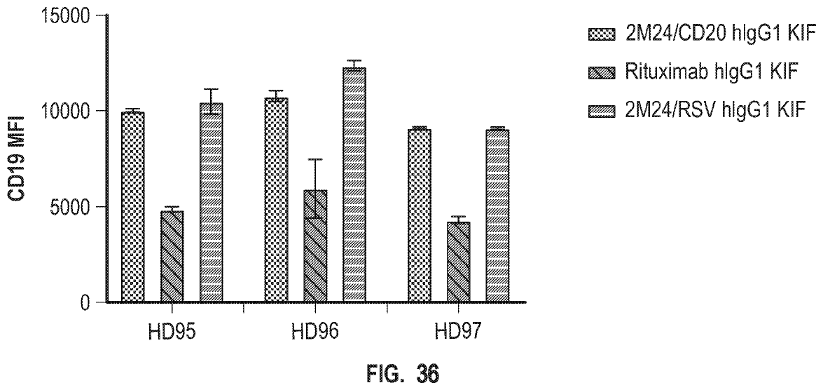

Next, the effect of the 2M24/CD20 bispecific on CD19 expression was examined in human B cells. Preserving target antigen expression is critical for therapeutic activity of monoclonal antibodies. B cell antigens such as CD20, CD19, and BCMA are validated immuno-oncology targets. CD19 is known to be downregulated via shaving/shedding following binding of anti-CD19 antibodies. Using CD20-targeting antibodies, a bystander effect was observed where CD19 expression was reduced upon treatment with Rituximab, but not with the 2M24/CD20 hIgG1 KIF bispecific (FIG. 36). CD19 levels on B cells were better maintained by 2M24/CD20 bispecific antibody compared to rituximab. Without wishing to be bound to theory, it is thought that, therapeutically, 2M24/CD20 bispecific may exhibit prolonged activity due to minimal impact on target antigen expression.

To compare rituximab with the anti-CD20 antibody obinutuzumab, 2M24 bispecific antibodies against CD20 were generated using the variable domain sequences from either Rituximab or Obinutuzumab. Obinutuzumab variable domain sequences were as follows. VH: QVQLVQSGAEVKKPGSSVKVSCKASGYAFSYSWINWVRQAPGQGLEWMGRIFPGDGD TDYNGKFKGRVTITADKSTSTAYMELSSLRSEDTAVYYCARNVFDGYWLVYWGQGTL VTVSS (SEQ ID NO:46); VL: DIVMTQTPLSLPVTPGEPASISCRSSKSLLHSNGITYLYWYLQKPGQSPQLLIYQMSNLVS GVPDRFSGSGSGTDFTLKISRVEAEDVGVYYCAQNLELPYTFGGGTKVEIK (SEQ ID NO: 47).

In an ADCC/ADCP assay, 2M24/CD20 (derived from Rituximab sequence) demonstrated almost complete depletion of B cells, superior to that of 2M24/CD20 (derived from obinutuzumab) or parental bivalent antibodies and isotype control (FIG. 37). These data support utilization of the rituximab sequence for 2M24/CD20 bispecific development

### Example 6: Characterization of a Bispecific Antibody Targeting Dectin-1 and CD20 in an Exploratory Study in Non-Human Primates

This Example describes the results of an exploratory study on the safety and efficacy of the bispecific antibody targeting human Dectin-1 and human CD20 described in Example 9 in cynomolgus monkeys.

### Materials and Methods

Three groups of Cynomolgus monkeys (1 male and 1 female per group) were treated with a single dose (5 mg/kg) of test articles: A) 2M24/CD20 hIgG1 KIF, B) 2M24/CD20 hIgG1 inert, and C) Rituximab hIgG1 KIF. Blood was collected at the indicated time points. Abbreviations for test articles (2M24/CD20 KIF, 2M24/CD20 inert, RTX KIF).

B cell levels were assessed by flow cytometry. Depletion was quantified by the number of CD19+ B cells remaining in samples post-dose compared to the levels before test-articles were administered. Bone marrow and lymph node aspirates were collected at the indicated time points, and B cell levels were assessed by flow cytometry. Depletion was quantified by the number of CD19+ B cells remaining in samples post-dose (Day 7) compared to the levels before test-articles were administered (Day −7).

For PBMC assay, PBMCs from a healthy Cyno were treated with a serial dilution of 2M24/CD20 hIgG1 KIF, Rituximab KIF, and isotype control RSV hIgG1 KIF antibodies. After 24 hours of treatment, PBMCs were stained with antibodies against lineage-specific markers for flow cytometry analysis. B cell depletion was quantified relative to the isotype control group.

### Results

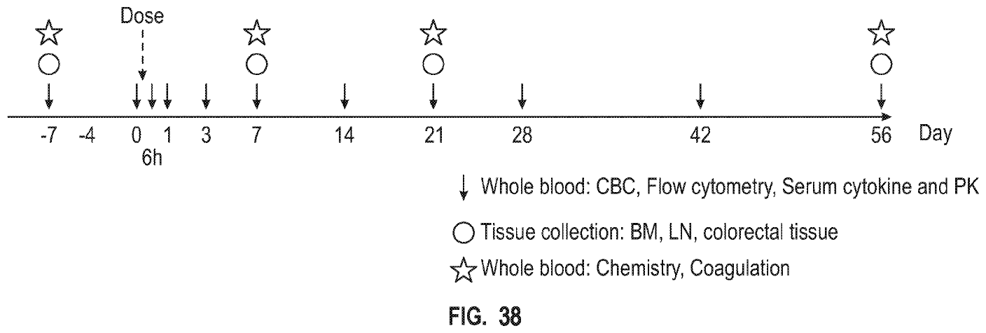

This exploratory study was designed to examine the safety and efficacy of 2M24/CD20 bispecific antibody in non-human primates. The study design is shown in FIG. 38. Cynomolgus monkeys were divided into three treatment groups comprising 2 animals (1 male, 1 female) per group. Each group was administered a specific test article at a single dose of 5 mg/kg. The test articles included: 1) 2M24/CD20 hIgG1 KIF, 2) 2M24/CD20 hIgG1 inert, and 3) Rituximab hIgG1 KIF. Animals were monitored daily, and samples such as whole blood, bone marrow, lymph node, and colorectal tissues were collected as indicated. The study was planned for 8 weeks.

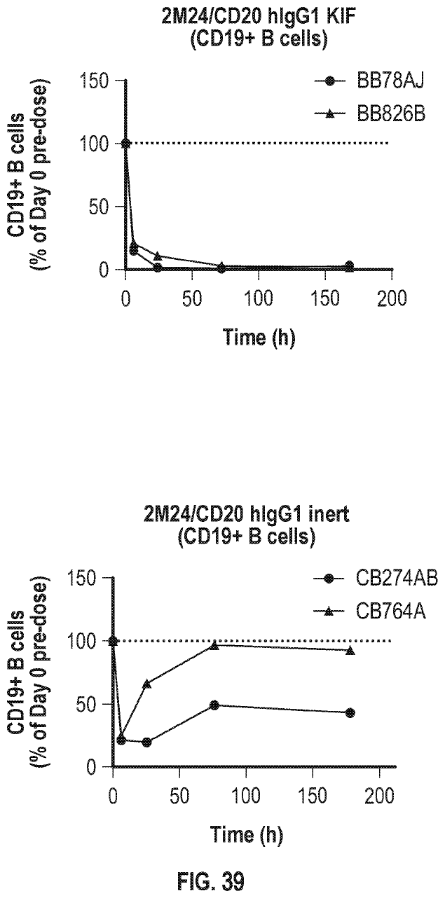

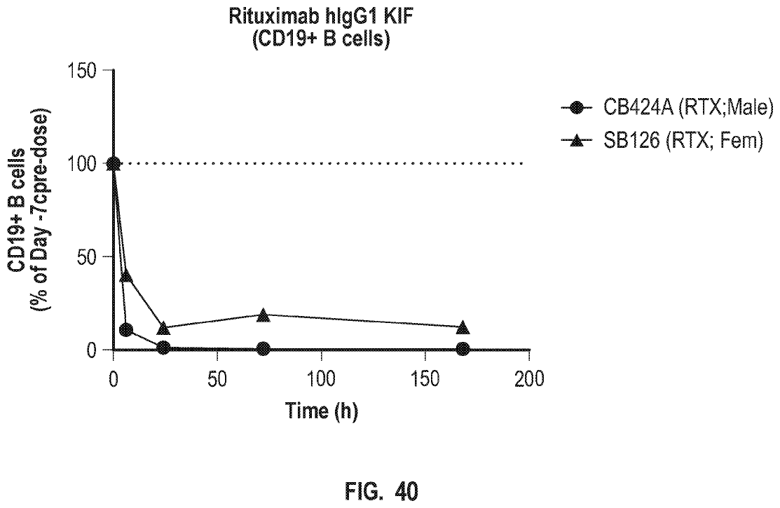

As shown in FIG. 39 (upper), 2M24/CD20 hIgG1 KIF bispecific antibody depleted B cells in vivo in Cynomolgus monkeys. Nearly complete and sustained B cell depletion (˜98%) was observed in both animals treated with a single dose (5 mg/kg) of 2M24/CD20 hIgG1 KIF. In the Rituximab group (FIG. 40), one animal showed complete depletion, whereas the second showed robust yet incomplete depletion (˜87%). Partial B cell depletion was observed for one animal in the 2M24/CD20 hIgG1 inert group (FIG. 39 lower), whereas the second animal showed no depletion at Day 7. 2M24/CD20 hIgG1 KIF bispecific antibody was well-tolerated in cynomolgus monkeys.

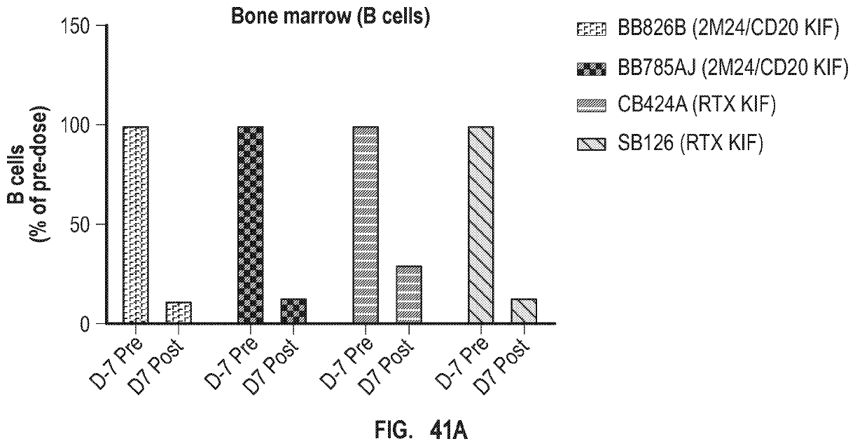

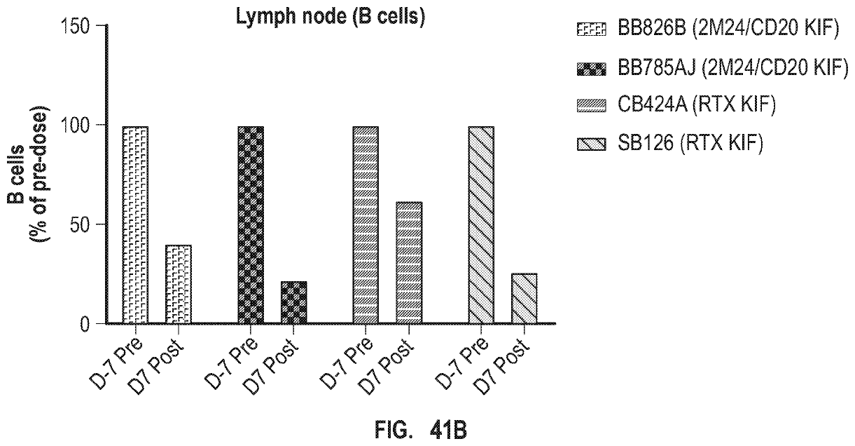

2M24/CD20 hIgG1 KIF bispecific also depleted bone marrow (FIG. 41A) and lymph node (FIG. 41B) B cells in vivo in Cynomolgus monkey. A single dose (5 mg/kg) of 2M24/CD20 hIgG1 KIF induced robust B cell depletion in the bone marrow (˜87-88%) and partial depletion in the lymph node (60-78%) in both animals. In the Rituximab group, B cell depletion was also observed in both tissues. Partial B cell depletion was observed in the 2M24/CD20 hIgG1 inert group, except for animal CB764A with minimal B cell depletion in the lymph node.

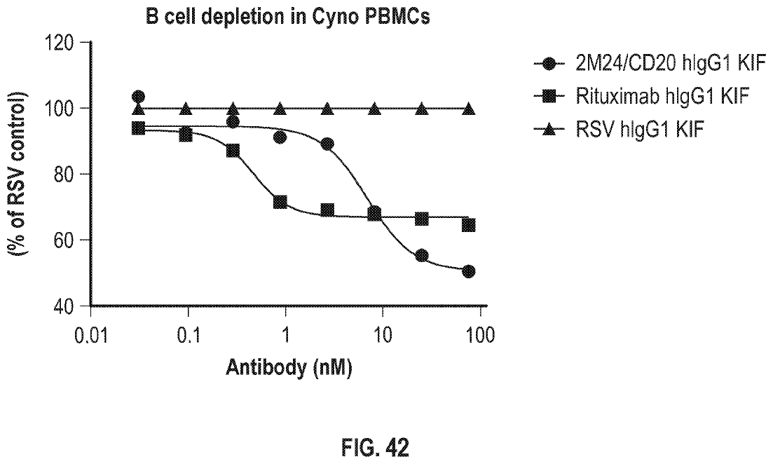

2M24/CD20 hIgG1 KIF bispecific antibody also induced robust depletion of Cyno B cells ex vivo (FIG. 42). 2M24/CD20 hIgG1 KIF induced robust depletion of B cells compared to Rituximab hIgG1 KIF. The maximum depletion achieved by Rituximab was ˜30% of B cells, whereas 2M24/CD20 hIgG1 KIF bispecific demonstrated maximum depletion at ˜50%.

### Example 7: Purification and Functional Characterization of the 2M24/CD20 Bispecific Antibody in Sc Fv Format

This Example describes the production, purification, and characterization of a 2M24/CD20 bispecific antibody in which the Dectin-1 targeting arm (based on 2M24 variable domains) was an scFv fused to a human IgG1 Fc domain with knob-forming mutations, and the CD20 targeting arm was based on rituximab hIgG1 with hole-forming mutations. A diagram of the molecule is shown in FIG. 43. Knob-forming mutation on Dectin-1 targeting arm was T366W; hole-forming mutations on CD20 targeting arm were T366S, L368A, and Y407V. Without wishing to be bound to theory, it is thought that this format provides a universal platform for generating anti-Dectin-1 bispecific antibodies with simpler manufacturing requirements (e.g., as compared to bispecific antibodies having an anti-Dectin-1 arm with multiple polypeptide chains).

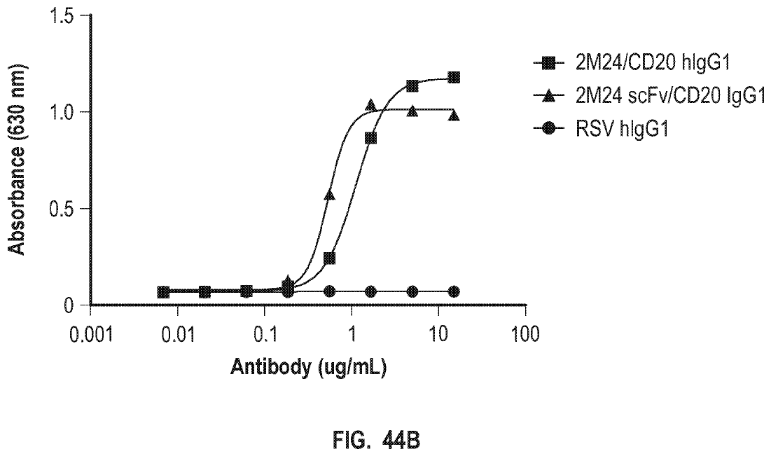

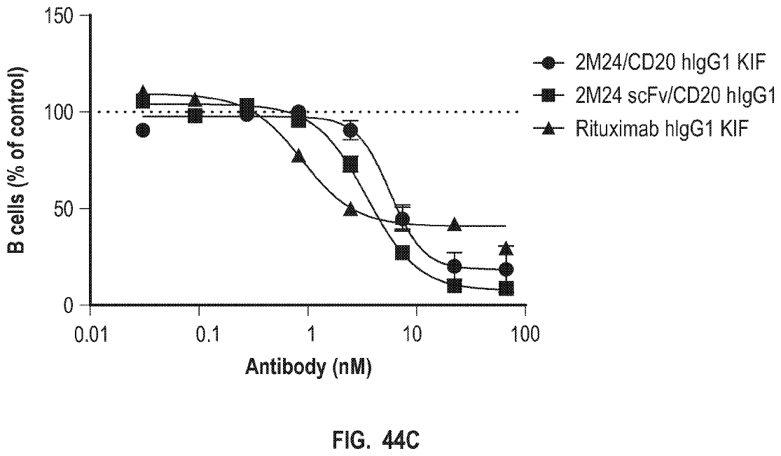

2M24 scFv/CD20 hIgG1 was expressed in Hek293 cells by transfecting 3 plasmids (2M24 scFv hIgG1 plasmid, CD20 heavy chain, and CD20 light chain). Supernatant was harvested after four days of expression and purified via Protein A. Aggregates were removed with size exclusion chromatography. As shown in FIG. 44A, the 2M24 scFv/CD20 hIgG1 bispecific antibody purified as a homogenous molecule on SEC.

Next, co-cultures of CD20-expressing Raji cells and the Dectin-1-expressing HEK reporter assay were treated with increasing concentration of 2M24 scFv/CD20 hIgG1 bispecific. Reporter activation was assessed by measuring SEAP levels (based on absorbance at 630 nm) in media. The bispecific molecule promoted targeted immune stimulation, as assessed by this NFkB reporter assay (FIG. 44B).

To examine B cell depletion, human PBMCs from a healthy donor were treated with a serial dilution of the indicated antibodies. After 24 hours of treatment, PBMCs were stained with antibodies against lineage-specific markers for flow cytometry analysis. B cells were quantified relative to an untreated control group (indicated by the dotted line in FIG. 44C). The results demonstrated that the 2M24 scFv/CD20 hIgG1 bispecific antibody, similar to the 2M24/CD20 hIgG1 KIF molecule, was able to deplete human B cells (FIG. 44C).

### Example 8: Characterization of B-Cell Depletion by 2M24×CD20 Bispecific Binding Protein

A non-fucosylated 2M24 scFv/CD20 bispecific binding protein with an anti-Dectin-1 single chain variable fragment (scFv) fused to a conventional anti-CD20 antibody arm with an hIgG1 Fc (see FIG. 43) was compared to the anti-CD20 antibody rituximab and a bispecific anti-CD3/anti-CD20 T-cell engager for ability to deplete B cells in healthy donor PBMCs. The non-fucosylated 2M24 scFv/CD20 bispecific binding protein comprised a first polypeptide chain comprising the amino acid sequence of SEQ ID NO:31, a second polypeptide chain comprising the amino acid sequence of SEQ ID NO:32, and a third polypeptide chain comprising the amino acid sequence of SEQ ID NO:33.

PBMCs were isolated from healthy donor buffy coats by Ficoll separation. PBMCs were resuspended in ADCC media (RPMI 1640, 10% heat-inactivating FBS, 1× pen/strep, 1× non-essential amino acids) at a density of 500,000 cells in 50 uL in round-bottom, ultralow adherent, 96-well plates. Test articles were prepared in ADCC media at 2× working solution, in a 3-fold serial dilution. 50 uL of test articles were added to cells. Reactions were gently resuspended and incubated at 37 C for 24 hours. Cells were collected, treated with human Fc block and live/dead dye, and then stained with for the following markers (CD45, CD3, CD16, CD14, CD56, and CD19). B cells were detected based on the phenotype CD45+CD3-CD14-CD56-CD19+. B cell levels were expressed as the percentage of CD19+ cells in treatment groups relative to CD19+ cells in the untreated control group.

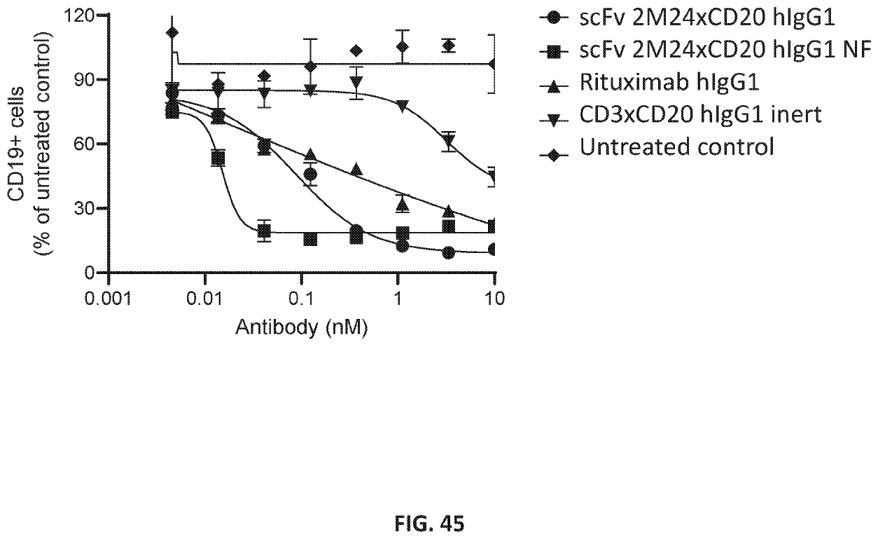

As shown in FIG. 45, the non-fucosylated scFv 2M24×CD20 bispecific binding molecule outperformed rituximab and the CD3×CD20 T cell engager in depleting B cells. These data indicate that a bispecific antibody targeting Dectin-1 on myeloid cells and CD20 on B cells was highly potent in depleting B cells compared to rituximab or a T cell engager. These findings suggest that engaging myeloid cells via Dectin-1 is an attractive cancer immunotherapy strategy.

Next, B cell depletion was assayed in a prostate cancer tumor biopsy.

Single-cell suspension was generated from a prostate cancer biopsy using the Tumor Dissociation kit by Miltenyi Biotec. CD20 expression was confirmed on CD45+CD3−CD14−CD56−CD19+ cells, and Dectin-1 expression was confirmed on CD45+CD3−CD19−CD11b+CD163+ tumor associated macrophages by flow cytometry. Cells were resuspended in ADCC media (RPMI 1640, 10% heat-inactivating FBS, 1× pen/strep, 1× non-essential amino acids) at a density of 500,000 cells in 100 uL in round-bottom, ultralow adherent, 96-well plates. Test articles were prepared in ADCC media at 3× concentration of 30 ug/mL, and 50 uL was added to cells (final concentration of 10 ug/mL). Reactions were gently resuspended and incubated at 37 C for 24 hours. Cells were collected, treated with human Fc block and live/dead dye, and then stained with for the following markers (CD45, CD3, CD16, CD14, CD11b, and CD19). B cells were detected based on the phenotype CD45+CD3−CD14−CD56−CD19+. B cell levels were expressed as the percentage of CD19+ cells in treatment groups relative to CD19+ cells in the control group (RSV hIgG1).

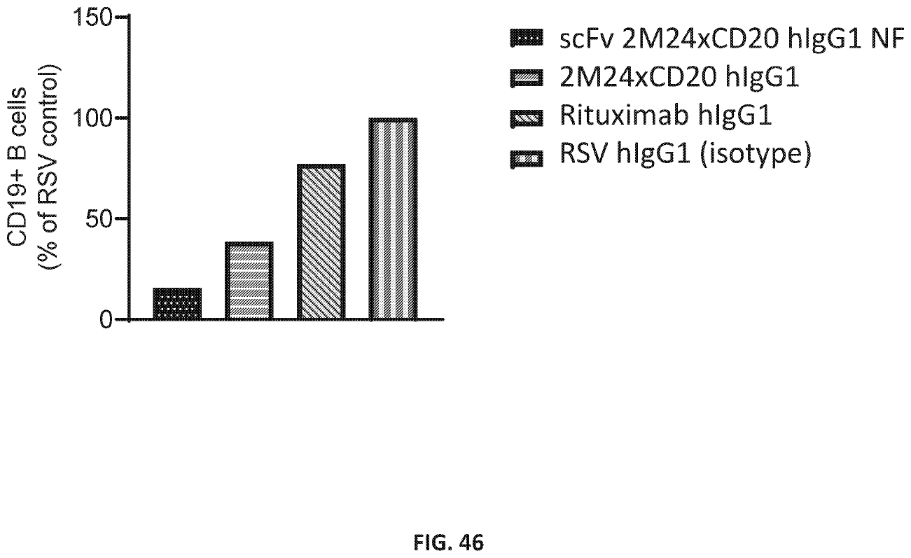

As shown in FIG. 46, the non-fucosylated scFv 2M24×CD20 bispecific binding molecule outperformed rituximab in depleting B cells. These data demonstrate that 2M24×CD20 was effective in depleting CD20-expressing B cells that are present in a solid tumor biopsy through engagement of Dectin-1-expressing tumor-associated macrophages. Moreover, the 2M24×CD20 bispecific, which comprised a scFv and non-fucosylated format, was superior to rituximab or the traditional IgG 2M24×CD20 format (DuetMab). Taken together, these observations confirm that engaging myeloid cells via Dectin-1 is an attractive cancer immunotherapy strategy, and that utilizing the scFv and non-fucosylated modifications can enhance potency.

### Example 9: Comparison of 2M24×CD20 Bispecific Binding Protein with Rituximab

The 2M24×CD20 bispecific binding protein was compared with rituximab for functional properties of interest, including depletion of target cells.

The effect of 2M24×CD20 bispecific binding protein on CD16 expression on NK cells was examined. Human PBMCs from a healthy donor were treated with a serial dilution of 2M24×CD20 bispecific hIgG1 binding protein treated with KIF, Rituximab treated with KIF, or isotype control RSV hIgG1 antibody treated with KIF. After 24 hours of treatment, PBMCs were stained with antibodies against lineage-specific markers for flow cytometry analysis. CD16 expression on CD56+ NK cells was quantified and compared to expression levels in the isotype control treated group.

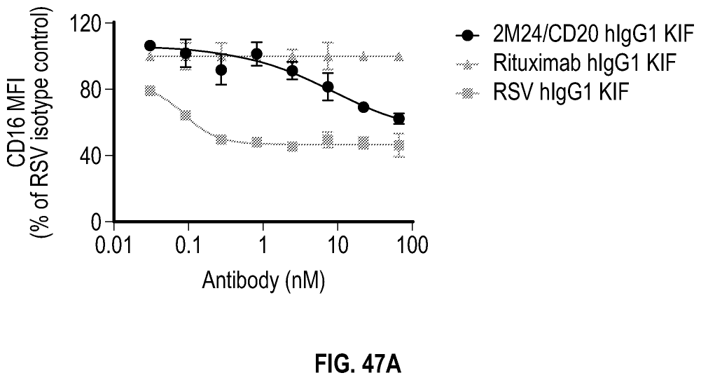

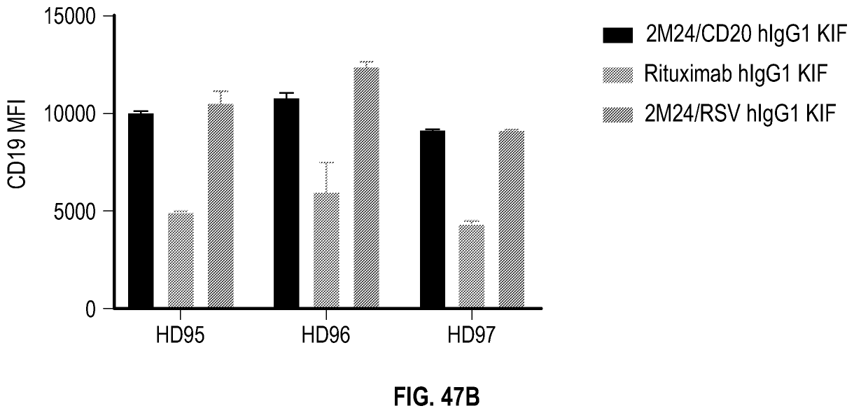

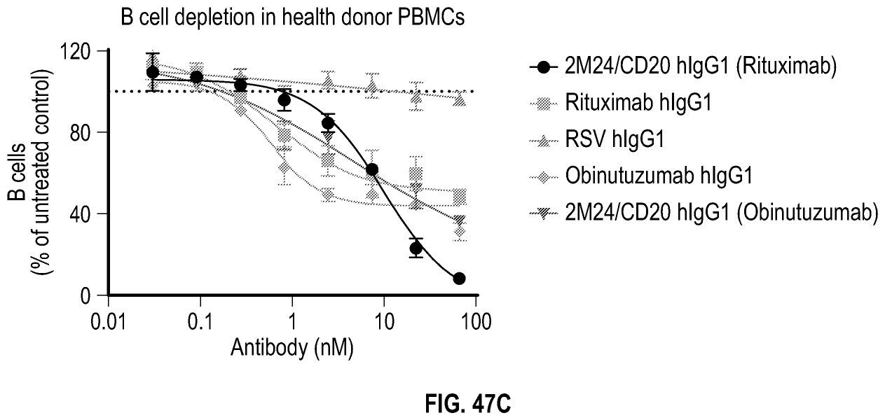

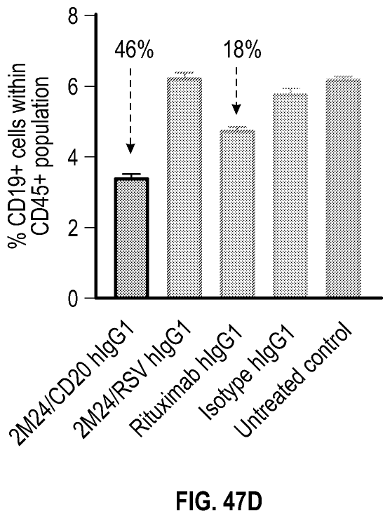

As shown in FIG. 47A, rituximab induced potent and robust shedding of CD16 on NK cells compared to the 2M24×CD20 bispecific hIgG1 binding protein (both treated with KIF). CD16 is required for ADCC activity by NK cells; therefore, loss of CD16 expression can decrease the cytotoxic potential of NK cells. These results suggest that the 2M24×CD20 bispecific binding protein has the potential to better preserve NK cell cytotoxic potential as compared to rituximab.

The effect of 2M24×CD20 bispecific binding protein on CD19 expression on B cells was examined. Human PBMCs from a healthy donor were treated with 0.1 nM of 2M24×CD20 bispecific hIgG1 binding protein treated with KIF, Rituximab treated with KIF, or isotype control RSV hIgG1 antibody treated with KIF. After 24 hours of treatment, PBMCs were stained with antibodies against lineage-specific markers for flow cytometry analysis. CD19 expression (MFI) on B cells was quantified.

Preserving target antigen expression is critical for therapeutic activity of monoclonal antibodies. B cell antigens such as CD20, CD19 and BCMA are validated immuno-oncology targets. CD19 is known to be downregulated via shaving/shedding following binding of anti-CD19 antibodies. Here, using CD20-targeting antibodies, a bystander effect was observed where CD19 expression was reduced upon treatment with KIF-treated rituximab, but not with KIF-treated 2M24×CD20 bispecific hIgG1 binding protein (FIG. 47B). Thus, CD19 levels on B cells were better maintained by 2M24×CD20 bispecific binding protein than rituximab. These results suggest that, therapeutically, 2M24×CD20 bispecific binding protein may exhibit prolonged activity due to minimal impact on target antigen expression.

The effect of 2M24×CD20 bispecific binding protein on B cell depletion was examined. Human PBMCs from a healthy donor were treated with a serial dilution of the indicated antibodies. After 24 hours of treatment, PBMCs were stained with antibodies against lineage-specific markers for flow cytometry analysis. B cells were quantified relative to an untreated control group (indicated by the dotted line in FIG. 47C).

2M24 bispecific binding proteins against CD20 were generated using the variable domain sequences from either rituximab or obinutuzumab. In an ADCC/ADCP assay, 2M24/CD20 bispecific (anti-CD20 arm derived from Rituximab sequence) demonstrated almost complete depletion of B cells compared to 2M24/CD20 (anti-CD20 arm derived from obinutuzumab) or parental bivalent antibodies and isotype control (FIG. 47C). Thus, 2M24×CD20 bispecific binding protein with anti-CD20 arm from rituximab showed better B cell depletion than a form using the anti-CD20 binding arm from obinutuzumab. These data support utilization of the rituximab variable domains for 2M24/CD20 bispecific development.

The effect of 2M24×CD20 bispecific binding protein on B cell depletion was also examined using a kidney cancer biopsy. A single-cell suspension was generated from kidney cancer biopsy, and the cells were treated with 2M24/CD20 bispecific hIgG1, 2M24/RSV bispecific hIgG1, Rituximab hIgG1, and isotype control RSV hIgG1 antibodies. After 24 hours of treatment, the cells were stained with antibodies against lineage-specific markers for flow cytometry analysis. B cells were quantified as the percentage of CD19+ cells within the CD45+ immune cell population.

The results showed that 2M24/CD20 bispecific binding protein induced robust depletion of B cells in a single-cell suspension from a primary kidney tumor (FIG. 47D). Dectin-1 expression on tumor-associated macrophages in the primary kidney biopsy was also confirmed. 2M24/CD20 bispecific induced superior B cell depletion as compared to rituximab.

### Example 10: Exploratory Study of 2M24×CD20 Bispecific Binding Protein in Cynomolgus Monkey

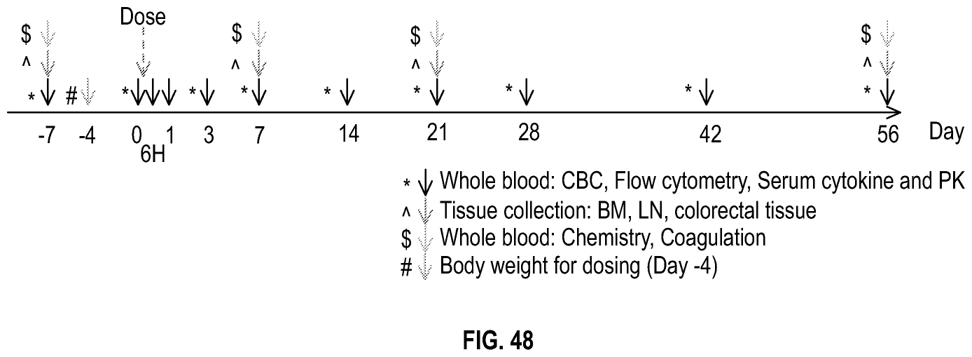

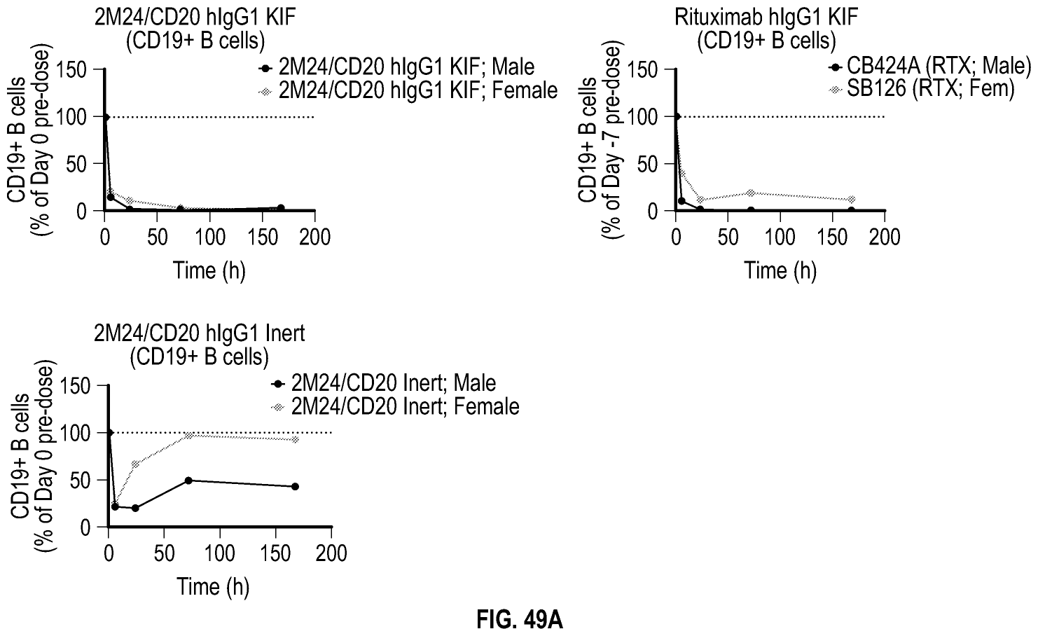

This Example describes the results of an exploratory study in cynomolgus monkey on the effect of 2M24/CD20 bispecific binding protein. The study design is illustrated in FIG. 48. Cynomolgus monkeys were divided into three treatment groups comprising 2 animals (1 male, 1 female) per group. Each group was administered a specific test article at a single dose of 5 mg/kg. The test articles include: 1) 2M24/CD20 hIgG1 KIF, 2) 2M24/CD20 hIgG1 inert, and 3) Rituximab hIgG1 KIF. Animals were monitored daily, and samples such as whole blood, bone marrow, lymph node, and colorectal tissues were collected as indicated. The study was planned for 8 weeks. Timepoints for sample collection and other measurements are shown in FIG. 48.

Blood B cell levels were examined. Blood was collected at the indicated time points, and B cell levels were assessed by flow cytometry. Depletion was quantified by the number of CD19+ B cells remaining in samples post-dose compared to the levels before test-articles were administered.

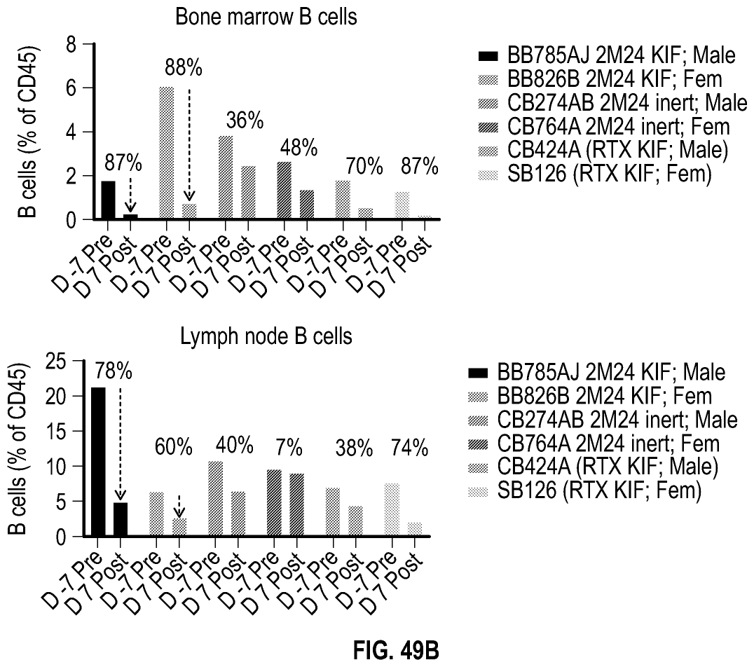

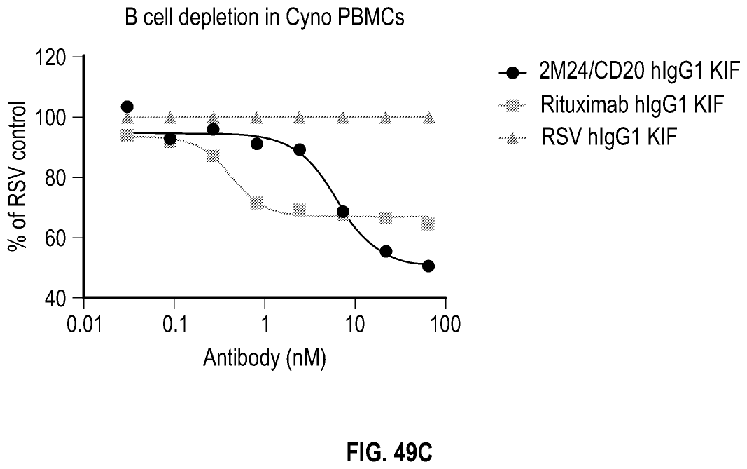

The results showed that 2M24/CD20 bispecific hIgG1 KIF depleted B cells in vivo. Nearly complete and sustained B cell depletion (˜98%) was observed in both animals treated with a single dose (5 mg/kg) of 2M24/CD20 hIgG1 KIF (FIG. 49A; upper left). In the Rituximab group, one animal showed complete depletion, whereas the second showed robust yet incomplete depletion (˜87%) (FIG. 49A; upper right). Partial B cell depletion was observed for one animal in the 2M24/CD20 hIgG1 inert group, whereas the second animals showed no depletion at Day 7 (FIG. 49A; lower left).

B cell depletion in bone marrow and lymph nodes was also examined. Bone marrow and lymph node aspirates were collected at the indicated time points, and B cell levels were assessed by flow cytometry. Depletion was quantified by the number of CD19+ B cells remaining in samples post-dose (Day 7) compared to the levels before test-articles were administered (Day-7).

The results showed that 2M24/CD20 bispecific hIgG1 KIF depleted bone marrow and lymph node B cells in vivo. A single dose (5 mg/kg) of 2M24/CD20 hIgG1 KIF induced robust B cell depletion in the bone marrow (˜87-88%) and partial depletion in the lymph node (60-78%) in both animals (FIG. 49B). In the Rituximab group, B cell depletion was also observed in both tissues. Partial B cell depletion was observed in the 2M24/CD20 hIgG1 inert group, except for animal CB764A with minimal B cell depletion in the lymph node.

Ex vivo B cell depletion was also examined. PBMCs from a healthy Cyno were treated with a serial dilution of 2M24/CD20 hIgG1 KIF, Rituximab KIF, and isotype control RSV hIgG1 KIF antibodies. After 24 hours of treatment, PBMCs were stained with antibodies against lineage-specific markers for flow cytometry analysis. B cell depletion was quantified relative to the isotype control group.

The results showed that 2M24/CD20 bispecific hIgG1 KIF induced robust depletion of cynomolgus B cells ex vivo. 2M24/CD20 bispecific hIgG1 KIF induced robust depletion of B cells compared to Rituximab hIgG1 KIF (FIG. 49C). The maximum depletion achieved by Rituximab was ˜30% of B cells, whereas 2M24/CD20 hIgG1 KIF bispecific demonstrated maximum depletion at ˜50%.

### Example 11: Surrogate Study of Anti-Dectin-1×CD20 Bispecific Binding Protein in Mouse

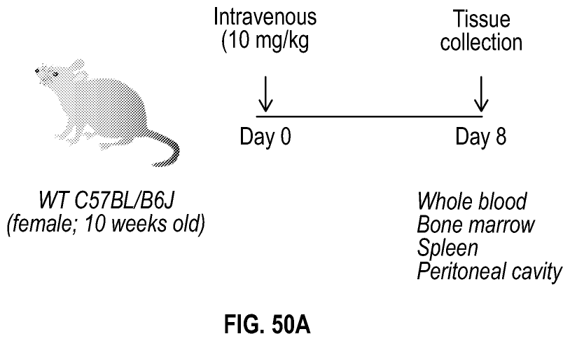

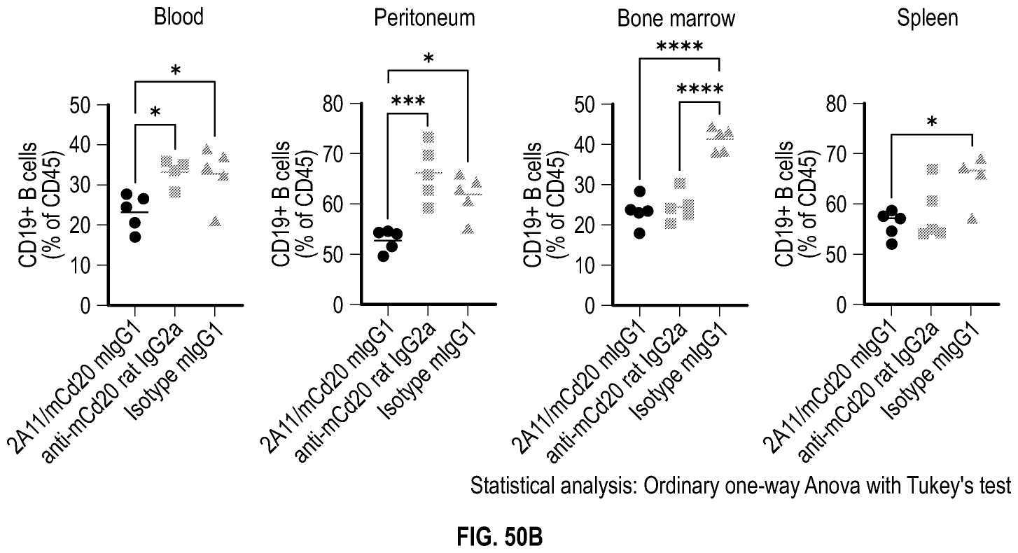

This Example describes the results of a surrogate study in mouse on the effect of 2A11 anti-mouse-Dectin-1/mCD20 bispecific binding protein. The study design is illustrated in FIG. 50A. Mice were treated with a single dose (10 mg/kg) of bispecific 2A11/mCd20 mIgG1 binding protein, bivalent anti-mCd20 rat IgG2a antibody, or a mIgG1 isotype control. Animals were sacrificed on Day 8, and tissues including blood, peritoneum, bone marrow and spleen were harvested for flow cytometry analysis. Single cell suspensions were stained with antibodies against lineage-specific markers. B cells were quantified based on Cd19 expression and reported as a % of total Cd45 cells.

As shown in FIG. 50B, the surrogate 2A11/mCd20 bispecific antibody induced significant depletion of B cells in blood and tissues of healthy, wild-type mice.

### Example 12: Testing of 2M24×CD20 Bispecific Binding Protein Efficacy Ex Vivo

Properties of the 2M24/CD20 bispecific binding protein were analyzed ex vivo. Ability of the 2M24/CD20 bispecific binding protein to activate the Dectin-1 pathway in the presence of CD20-expressing B cell lymphoma lines was examined. B cell lymphoma lines with different CD20 expression levels were treated with a serial dilution of the indicated antibodies in presence of Dectin-1 expressing reporter HEK cell line. After 24 hours of treatment, supernatant was collected to test the Dectin-1 induced NF-kB driven secreted embryonic alkaline phosphatase (SEAP) activity. D-zymosan, an established ligand of Dectin-1 was used as a positive control.

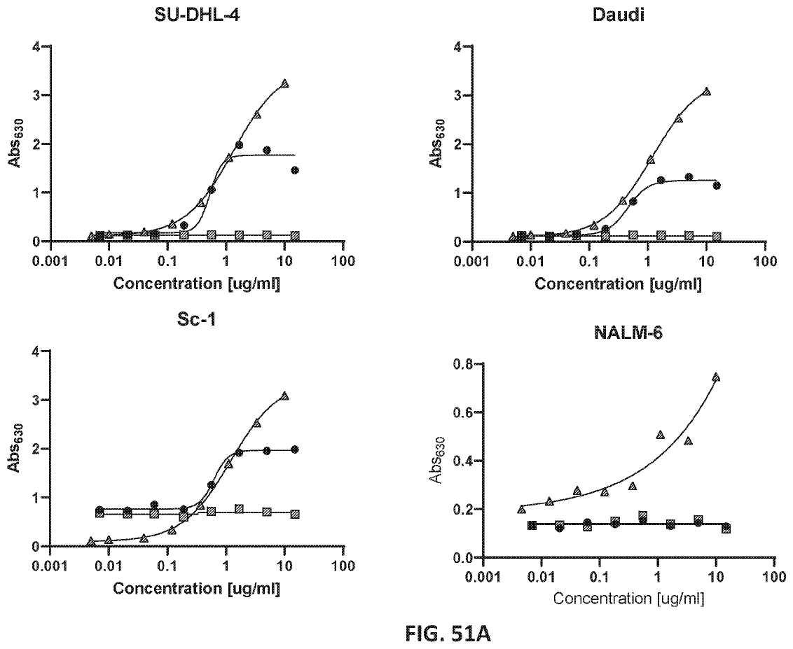

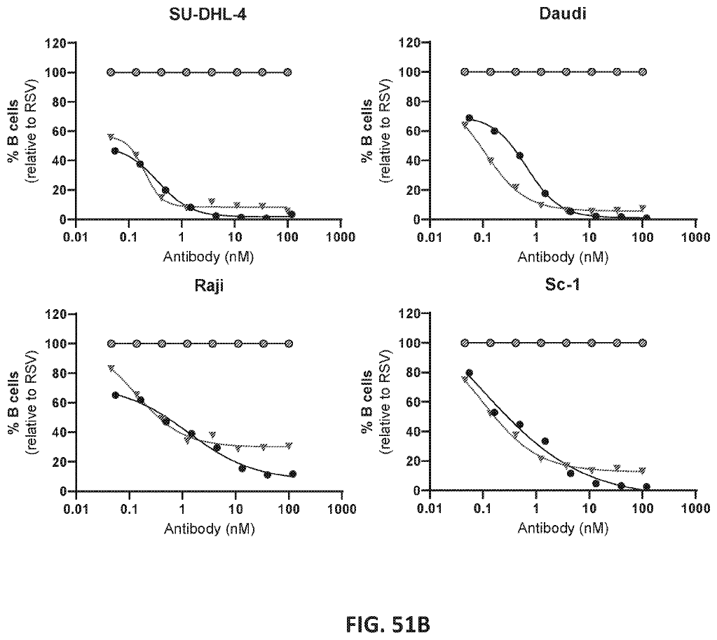

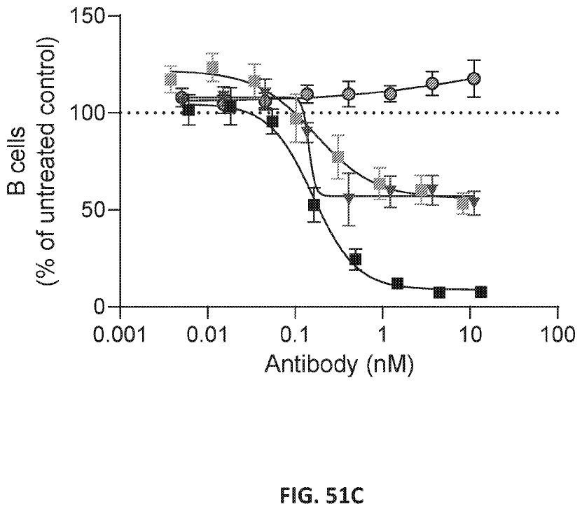

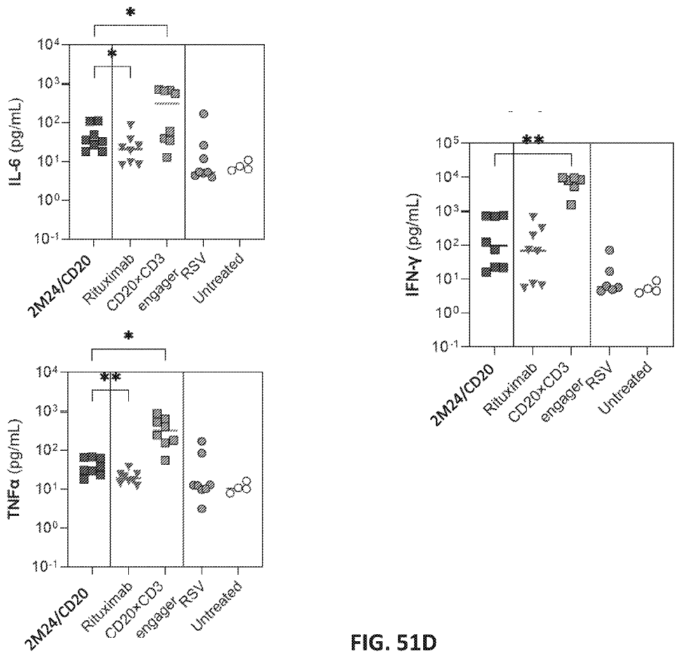

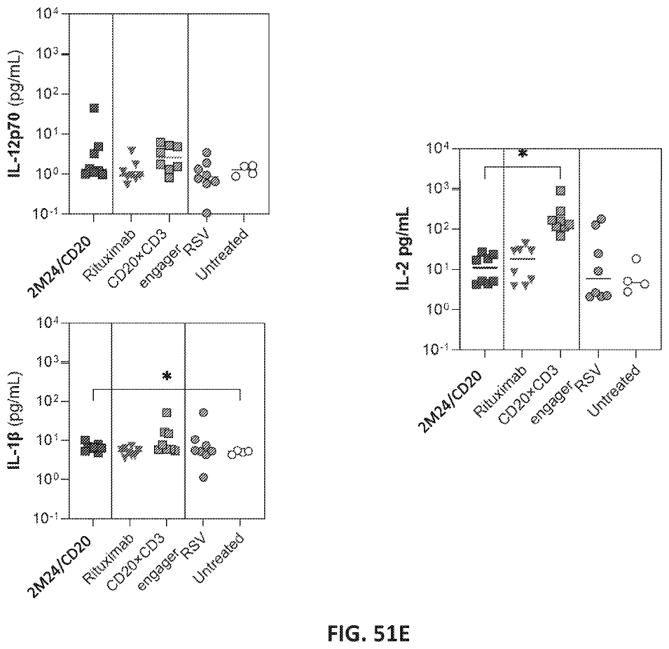

The results indicated that 2M24/CD20 bispecific binding protein induced Dectin-1 pathway activation only in the presence of CD20-expressing B cells. As shown in FIG. 51A, 2M24/CD20 bispecific binding protein showed dose-dependent activation of NF-kB in presence of B lymphoma lines expressing CD20 levels ranging from 9,000 (Sc-1) to 370,000 copies (SU-DHL-6). However, 2M24/CD20 bispecific binding protein failed to activate NF-kB reporter cell in CD20-null B lymphoma line (NALM-6) or in absence of any target cells. D-zymosan showed reporter HEK cell Dectin-1 pathway activation regardless the presence of any target cells. Together, these data indicate that simultaneous binding of Dectin-1 and engagement of CD20 (as low as 9,000 copies) on target cells is required for Dectin-1 clustering and activation of downstream NF-kB pathway.

The ability of 2M24/CD20 bispecific binding protein to induce phagocytosis was also tested. B cell lymphoma lines with different CD20 expression levels were treated with a serial dilution of the indicated antibodies in presence of healthy human donor derived macrophage cells. After 24 hours of treatment, B cell depletion was evaluated by relative to RSV (non-specific)-treated control group.

The results indicated that 2M24/CD20 bispecific binding protein induced more efficient depletion of B cell lymphoma cell lines in the presence of macrophage cells than rituximab. As shown in FIG. 51B, 2M24/CD20 bispecific binding protein treatment led to a dose-dependent increase in the level of depletion of B lymphoma lines in presence of healthy human donor-derived macrophage cells. In comparison to rituximab, 2M24/CD20 bispecific binding protein at the highest concentration showed more efficient or complete depletion of the B lymphoma lines with CD20 expression ranging from 9,000 to 370,000 receptors.

The ability of 2M24/CD20 bispecific binding protein to deplete primary human B cells was also tested. Human PBMCs from n=6 healthy donors were treated with a serial dilution of 2M24/CD20 bispecific binding protein and comparator anti-CD20 antibodies (rituximab and anti-CD20/anti-CD3 bispecific engager). 24 hours following the treatment, the PBMCs were stained with fluorophore labelled antibodies for flow cytometry analysis. Degree of B cell depletion was evaluated relative to an untreated control.

The results indicated that 2M24/CD20 bispecific binding protein showed more efficient depletion of primary human B cells compared to anti-CD20 antibody rituximab or anti-CD20/anti-CD3 bispecific T cell engager. 2M24/CD20 bispecific binding protein showed dose-dependent increase in the level of depletion of primary human B cells, with an average EC50 of 0.16 (FIG. 51C). In comparison, the other anti-CD20 antibodies tested, rituximab, and CD20×CD3 bispecific engager induced B cell depletion with an average EC50 values of 0.14 and 0.17 nM. However, whereas, 2M24/CD20 bispecific binding protein showed ˜91% depletion of primary B cells at the highest concentration, rituximab and CD20×CD3 engager showed a partial depletion of 46% and 47% of total B cells, respectively. Together, these data indicate that 2M24/CD20 bispecific binding protein demonstrated more robust B cell depletion compared to rituximab and CD20×CD3 engager.

The ability of 2M24/CD20 bispecific binding protein to stimulate cytokine secretion ex vivo was also tested. Human PBMCs from n=8 healthy donors were treated with a serial dilution of 2M24/CD20 bispecific binding protein and comparator anti-CD20 antibodies. 24 hours following the treatment, the supernatant was collected and analyzed by multiplex MSD cytokine assay to determine and compare the cytokine secretion induced by 2M24/CD20 bispecific binding protein and the comparator anti-CD20 antibodies. Supernatants from RSV hIgG1 (non-specific antibody)-treated and untreated PBMC samples were used as negative control.

The results indicated that 2M24/CD20 bispecific binding protein showed low to moderate production of pro-inflammatory cytokines. 2M24/CD20 bispecific binding protein showed dose-dependent induction in the cytokine production following 24-hour treatment with healthy donor PBMCs. At the highest concentration tested, 1.67 ug/ml, 2M24/CD20 bispecific binding protein showed significant induction of IL-6, TNFa, IFN-g, and IL-2 when compared to untreated controls (FIGS. 51D & 51E). Specifically, 2M24/CD20 bispecific binding protein showed a higher production of cytokines IL-6, and TNF-a compared to rituximab; however, the level of the 2M24/CD20 bispecific binding protein induced production of IL-6, TNF-a, IFN-g, and IL-2 was significantly lower as compared to the treatment with CD20×CD3 engager.

### Example 13: Testing of 2M24×CD20 Bispecific Binding Protein Efficacy in an In Vivo Mouse Model

Efficacy of a surrogate anti-mouse Dectin-1 antibody with mIgG2a Fc region was tested in an in vivo mouse model.

First, anti-tumor activity of the anti-mouse Dectin-1/anti-CD20 (rituximab) mIgG2a antibody was tested in several B cell lymphoma xenograft models. Surrogate anti-mDectin-1/anti-hCD20 mIgG2a antibody efficacy was evaluated in Ramos and Daudi xenograft models in SCID mice, and Raji xenograft model in SCID-beige mice. Following the corresponding tumor establishment in the SCID or SCID-beige mice, when tumor volume reached a range of 60-100 mm3, 4 doses of anti-mDectin-1/anti-mCD20 mIgG2a was administered at an equivalent dose of 10 mg/kg at days 0, 4, 7 and 10. The tumor progression was monitored by measuring the tumor volume twice a week and compared to isotype mIgG2a treatment.

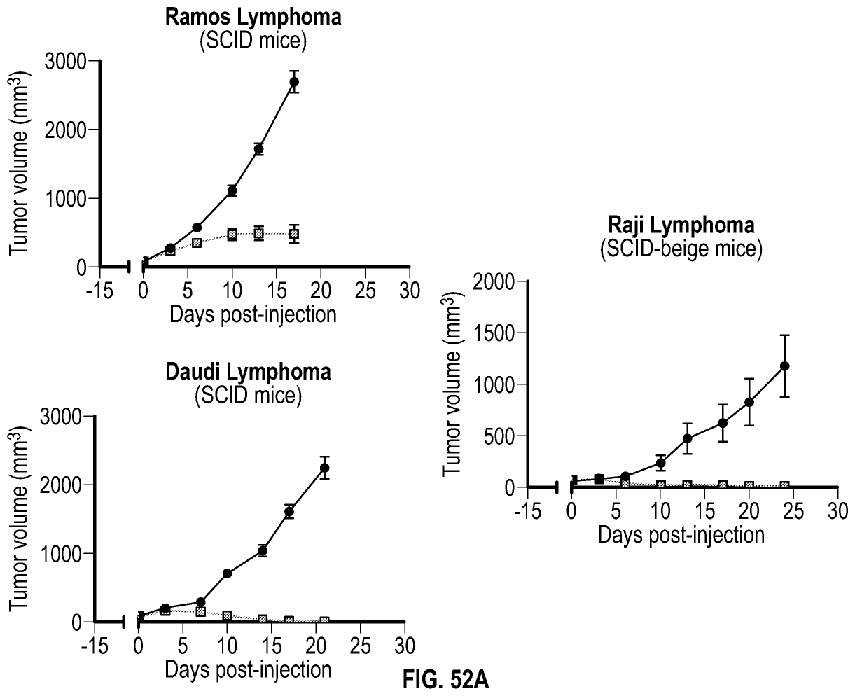

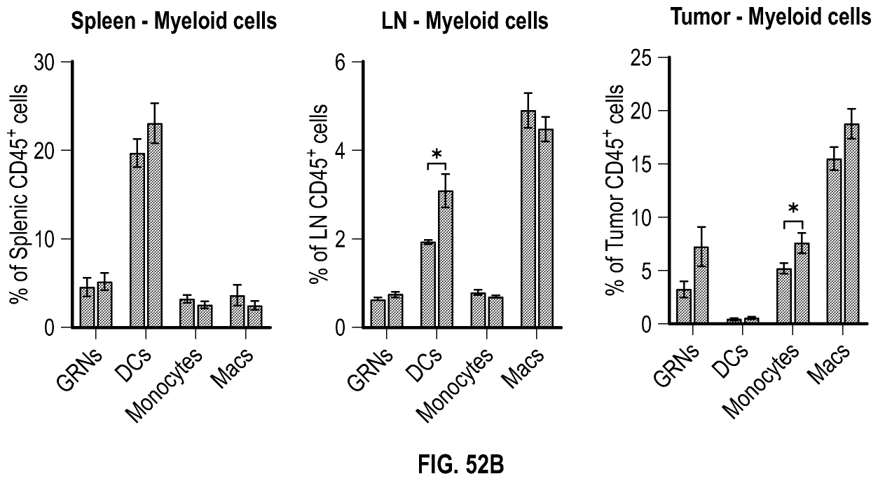

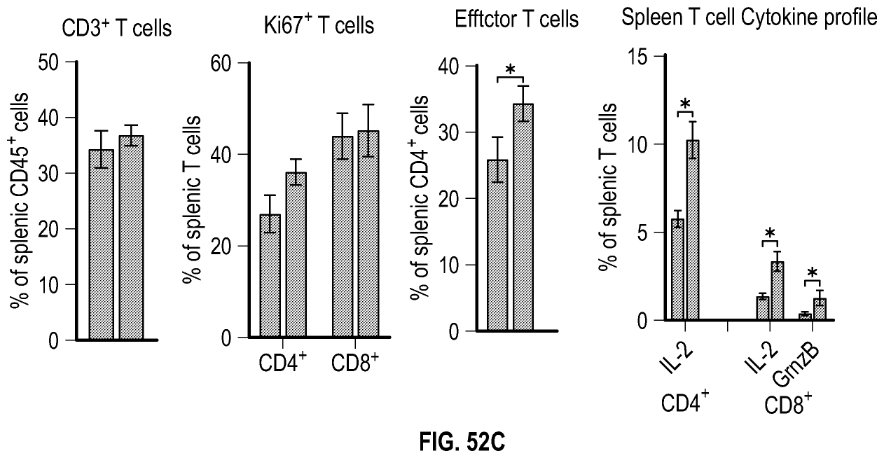

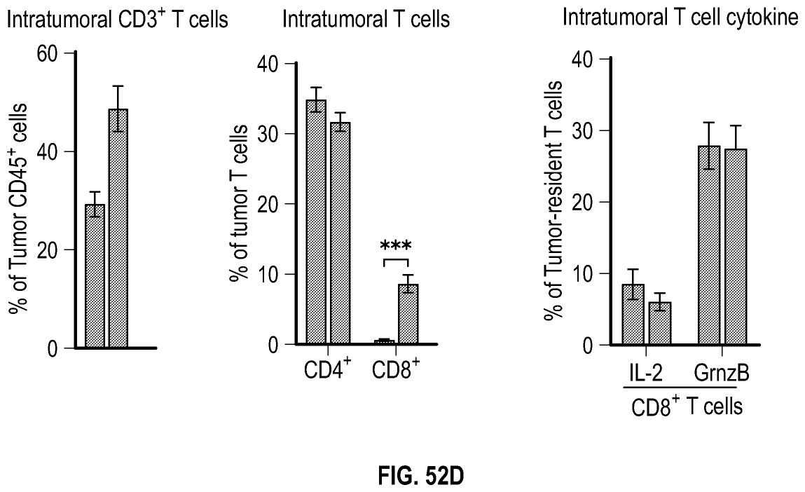

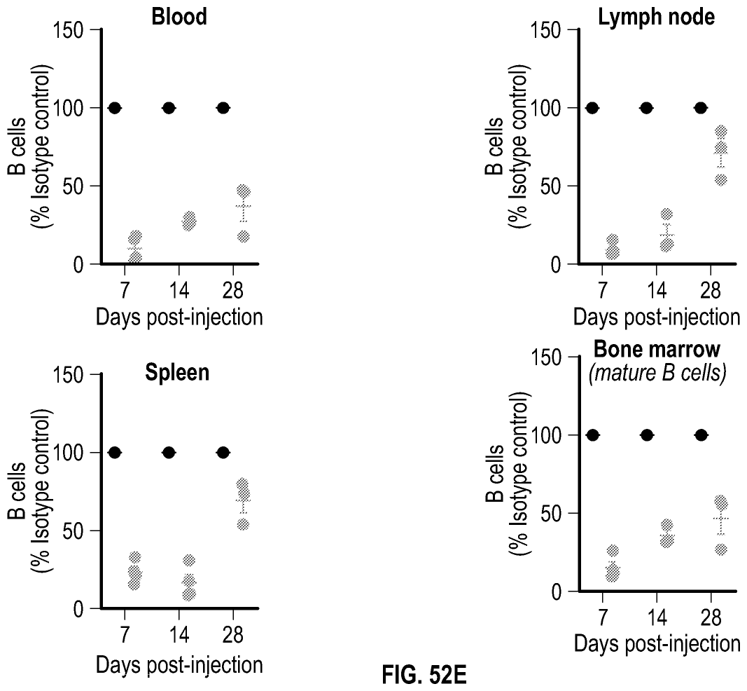

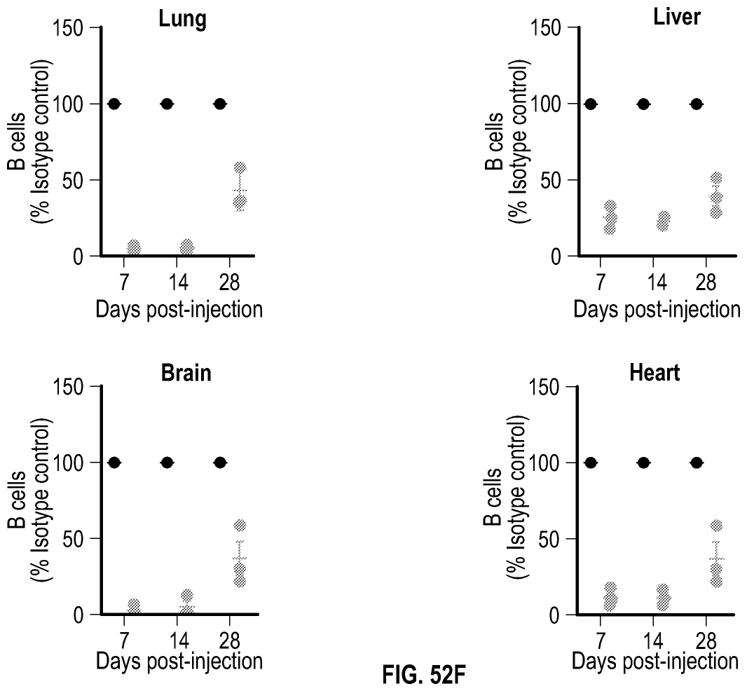

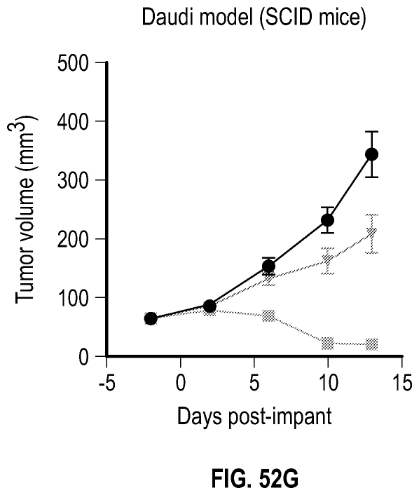

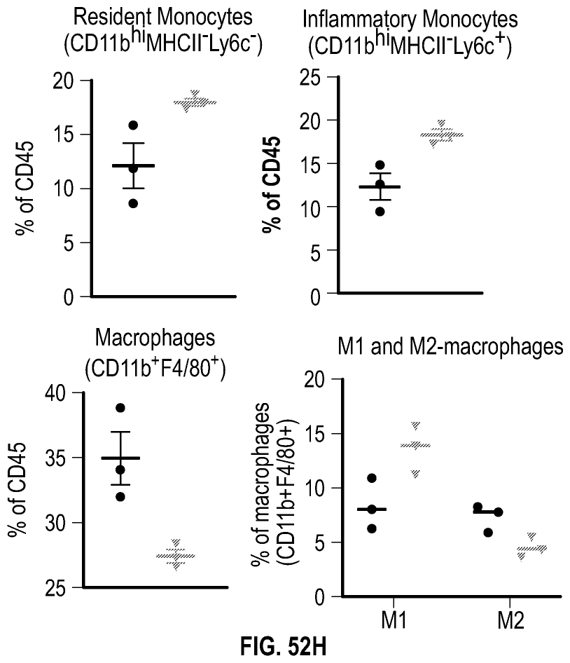

The results indicated that the surrogate anti-mDectin-1/anti-mCD20 mIgG2a antibody showed robust anti-tumor efficacy across several B cell lymphoma xenograft models. Four doses of 10 mg/kg administration of surrogate anti-mDectin-1/anti-mCD20 mIgG2a demonstrated 90% tumor growth inhibition for Ramos xenograft model is SCID mice, and complete tumor regression in Daudi model in SCID mice (10/10), and Raji model in SCID-beige mice (8/8) (FIG. 52A). Complete tumor regression in SCID mice and specifically NK cell-deficient SCID-beige mice indicated anti-mDectin-1/anti-mCD20 efficacy in myeloid cell activation in resolution of tumor growth in these mouse xenograft models.

Next, ability of the surrogate anti-mDectin-1/anti-mCD20 bispecific antibody to induce myeloid cell activation was tested in immunocompetent mouse models. Murine colon-carcinoma cell line MC38 was modified to express human CD20, and 5×10\hat{ }6 CD20-MC38 cells were used to implant (s.c.) in C57BL/6 mice. Following tumor establishment, when tumor volume reached a range of 80-150 mm3, single dose of anti-mDectin-1/anti-mCD20 bispecific was administered at an equivalent dose of 10 mg/kg and compared against similar doses of isotype mIgG1 treatment (n=10 per group). At 8 days following the treatment, the spleen, lymph node (LN), and tumor were analyzed by flow cytometry to assess myeloid cell activation.

The results indicated that anti-mDectin-1/anti-mCD20 bispecific antibody induced activation of dendritic cells in the lymphoid organs (spleen and lymph nodes), as well as macrophage polarization in the tumor. As shown in FIG. 52B, single administration of anti-mDectin-1/anti-mCD20 bispecific mIgG1 at 10 mg/kg showed significantly higher dendritic cell expansion in LN compared to isotype treated control group. In the tumor tissue, anti-mDectin-1/anti-mCD20 bispecific-induced myeloid cell activation was observed by a significantly higher influx of monocytes in the tumor, along with an improved polarization towards pro-inflammatory M1 macrophages, when compared to the isotype treated control.

T cell activation induced by anti-mDectin-1/anti-mCD20 bispecific antibody was also indicated in the MC38 xenograft model. Following CD20-MC38 tumor establishment in C57BL/6 mice, at a TV of 80-150 mm³, single dose of anti-mDectin-1/anti-mCD20 bispecific antibody was administered at an 10 mg/kg and compared against isotype mIgG1 and anti-CD20 mIgG1 treatment. At 8 days following the treatment, the spleen and tumor were analyzed by flow cytometry to assess T cell activation.

The results indicated that anti-mDectin-1/anti-mCD20 bispecific antibody led to activation of CD4+ and CD8+ T cells in the spleen, as well as intra-tumoral activation of cytotoxic CD8+ T cells in the tumor. As shown in FIG. 52C, single administration of anti-mDectin-1/anti-mCD20 bispecific mIgG1 at 10 mg/kg showed significantly higher T cell expansion in spleen, as indicated by the proliferation marker Ki67 staining. The significantly higher expansion of both T cell subsets coincided with an improved production of IL-2 in both T cell subsets, higher CD4⁺ effector T cell formation, and higher production of granzyme B by CD8⁺ T cells, indicating their cytotoxic capacity. In the tumor tissue, anti-mDectin-1/anti-mCD20 bispecific induced significantly higher influx of T cells, specifically CD8⁺ T cells (FIG. 52D). Furthermore, intra-tumoral CD8⁺ T cells showed robust granzyme B expression in anti-mDectin-1/anti-mCD20 bispecific-induced mice, indicating their cytotoxic nature. Taken together, anti-mDectin-1/anti-mCD20 bispecific demonstrated a robust T cell activation in the lymphoid organs, and intra-tumoral infiltration of cytotoxic CD8⁺ T lymphocytes.

Ability of anti-mDectin-1/anti-mCD20 bispecific antibody to deplete B cells in lymphoid organs of naïve C57BL/6 mice was tested. hCD20 Tg mice (hCD20 expressed under the mouse CD20 promoter) were treated with a single dose of anti-mDectin-1/anti-mCD20 bispecific mIgG2a and isotype mIgG2a control. The mice were sacrificed at day 7, 14 and 28 following the treatment. Indicated lymphoid and non-lymphoid organs were harvested to determine the B cell depletion relative to the isotype treated controls. B cell populations were marked by the double expression of CD19 and B220 and identified by flow cytometry.

The results indicated that anti-mDectin-1/anti-mCD20 bispecific showed robust depletion of B cells in the periphery and in lymphoid organs (spleen, lymph node, and bone marrow). Single administration of anti-mDectin-1/anti-mCD20 bispecific mIgG2a at 10 mg/kg showed significant depletion of B cells in the periphery and lymphoid organs. At day 7 post-treatment; ˜90% B cell depletion was observed in the periphery as well as in secondary lymphoid organs—spleen and lymph node (FIG. 52E). In bone marrow, the mature B cells (showing high CD20 expression) showed ˜85% depletion by day 7 post-treatment. Immature B cells with low CD20 expression showed more resistance to anti-mDectin-1/anti-mCD20 bispecific induced cell depletion (not shown). B cell recovery was monitored slowly over the course of the time following the single dose treatment.

Ability of anti-mDectin-1/anti-mCD20 bispecific antibody to deplete B cells in non-lymphoid organs of naïve C57BL/6 mice was also tested. hCD20 Tg mice (hCD20 expressed under the mouse CD20 promoter) were treated as described above. Indicated non-lymphoid organs were harvested to determine the B cell depletion relative to the isotype treated controls. Non-lymphoid tissues were processed into single-cell populations and enriched for immune cells. B cell populations were marked by the double expression of CD19 and B220 and identified by flow cytometry.

The results indicated that anti-mDectin-1/anti-mCD20 bispecific antibody showed robust depletion of B cells in the non-lymphoid tissues tested. Single administration of anti-mDectin-1/anti-mCD20 bispecific mIgG2a at 10 mg/kg showed robust depletion of B cells in the non-lymphoid organs (FIG. 52F). At day 7 post-treatment, near complete depletion of B cells was observed non-lymphoid organs-lung, liver, brain and heart. B cell recovery was monitored, and even at 28 days following the single-dose treatment, B cells only recovered to 50% of the isotype treated controls.

Ability of anti-mDectin-1/anti-mCD20 bispecific antibody with inert Fc to inhibit tumor growth was tested. Efficacy of mouse surrogate anti-mDectin-1/anti-mCD20 bispecific antibody with mIgG2a active and inert Fc regions was evaluated in Daudi xenograft models in SCID mice. Following the tumor establishment in the SCID, when tumor volume reached a range of 60-100 mm³, 4 doses of anti-mDectin-1/anti-mCD20 bispecific were administered at an equivalent dose of 10 mg/kg at days 0, 4, 7 and 10. The tumor progression was monitored by measuring the tumor volume twice a week and compared to isotype mIgG2a treated control. Tumors from anti-mDectin-1/anti-mCD20 bispecific Fc-inert treated group and isotype control group were further evaluated for myeloid cell activation at day 14 following the initial dosing; anti-mDectin-1/anti-mCD20 bispecific active group showed complete tumor regression, and hence was not included in the assessment.

The results indicated that anti-mDectin-1/anti-mCD20 bispecific with inert Fc still demonstrated robust tumor growth inhibition in SCID mice and induced an improved immune cell influx in the tumor tissue, highlighting Dectin-1-dependent activity. Treatment with anti-mDectin-1/anti-mCD20 bispecific mIgG2a (active) and Fc-inert induced 95% and 40% tumor growth regression and inhibition, respectively, compared to isotype mIgG2a control in Daudi model (FIG. 52G). Data indicate that targeting Dectin-1 alone is sufficient to achieve tumor growth inhibition; however, engaging both Dectin-1 and Fc receptors contribute to significant tumor elimination.

Ability of anti-mDectin-1/anti-mCD20 bispecific with inert Fc to reprogram myeloid cells was also tested. Mouse surrogate anti-mDectin-1/anti-mCD20 bispecific mIgG2a active and Fc-inert efficacy were evaluated in Daudi xenograft models in SCID mice. Following the tumor establishment in the SCID, when tumor volume reached a range of 60-100 mm3, 4 doses of anti-mDectin-1/anti-mCD20 bispecific were administered at an equivalent dose of 10 mg/kg at days 0, 4, 7 and 10. The tumor progression was monitored by measuring the tumor volume twice a week and compared to isotype mIgG2a treated control. Tumors from anti-mDectin-1/anti-mCD20 bispecific Fc-inert treated group and isotype control group was further evaluated for myeloid cell activation at day 14 following the initial dosing; anti-mDectin-1/anti-mCD20 bispecific active group showed complete tumor regression, and hence was not included in the assessment.

The results indicated that anti-mDectin-1/anti-mCD20 bispecific Fc inert-induced Dectin-1 stimulation drove considerable myeloid reprogramming in tumors with higher monocyte recruitment and pro-inflammatory macrophage differentiation. Four doses of 10 mg/kg administration of anti-mDectin-1/anti-mCD20 bispecific mIgG2a Fc-inert demonstrated a significantly improved recruitment of resident and inflammatory monocytes to the tumor tissue, as compared to isotype treated control group (FIG. 52H). While the frequency of intra-tumoral macrophages was lower in anti-mDectin-1/anti-mCD20 bispecific Fc-inert treated group compared to control, macrophages shows better polarization towards inflammatory M1 phenotype rather than tumorigenic M2 phenotype, indicating their anti-tumor efficacy.

### Example 14: Testing of 2M24×CD20 Bispecific Binding Protein Efficacy in an In Vivo Cynomolgus Model

Efficacy of 2M24/CD20 bispecific binding protein was tested in an in vivo cynomolgus monkey model.

B cell depletion in response to various doses of 2M24/CD20 bispecific binding protein was analyzed. Four groups of naïve cynomolgus monkeys (n=2; 1 male and 1 female in each group) were treated with three weekly doses of vehicle, and 1, 10 and 100 mg/kg of 2M24/CD20 bispecific binding protein. Following the dose regimen, samples were collected at several timepoints from peripheral blood from 1 and 10 mg/kg groups to assess the presence of B cells by flow cytometry. Vehicle and 100 mg/kg group animals were sacrificed at day 15, one-day after the third dose. B cells were marked by the double expression of HLA-DR and CD19.

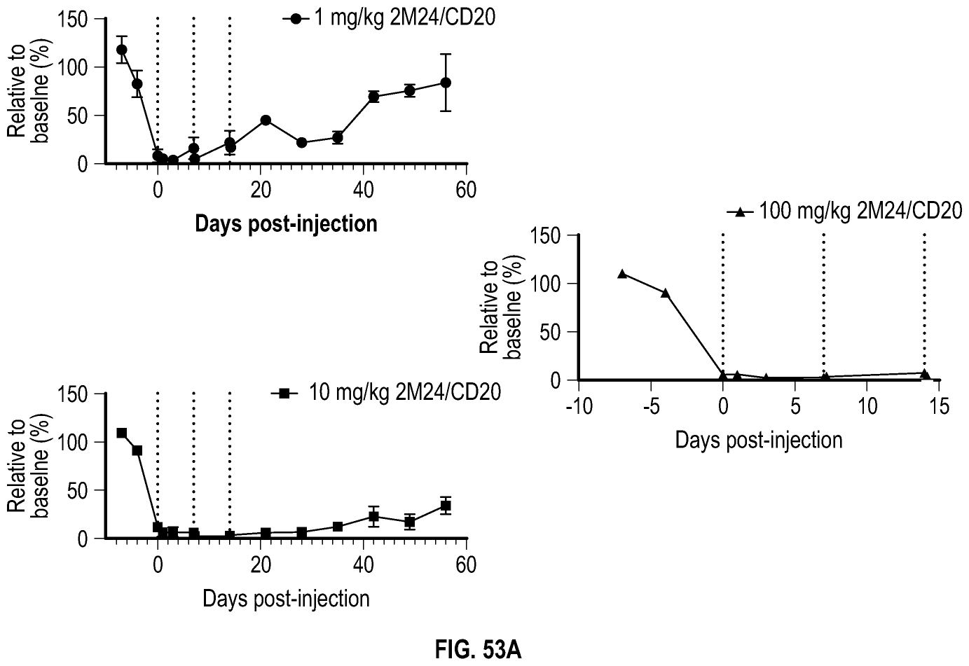

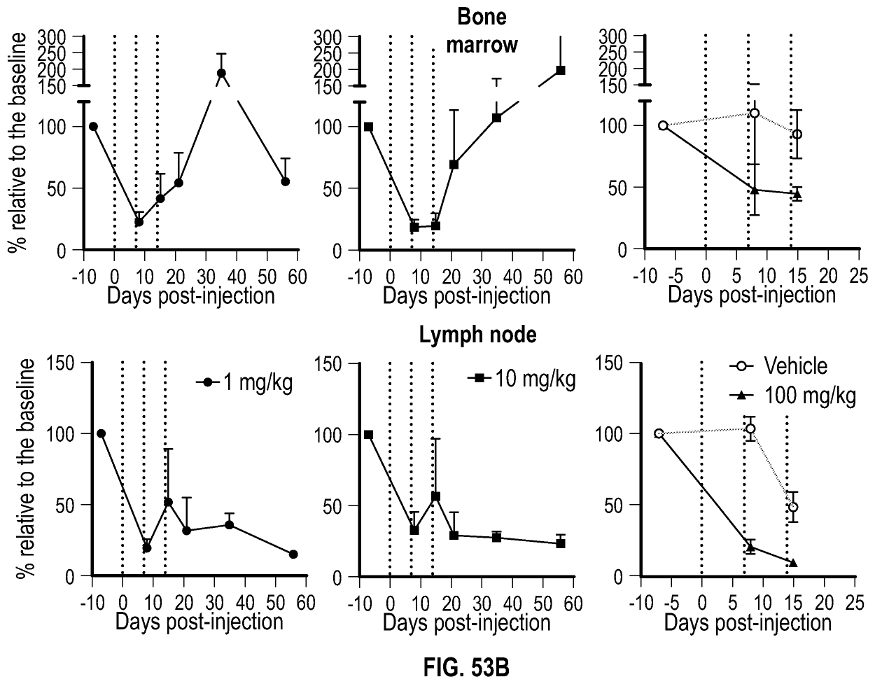

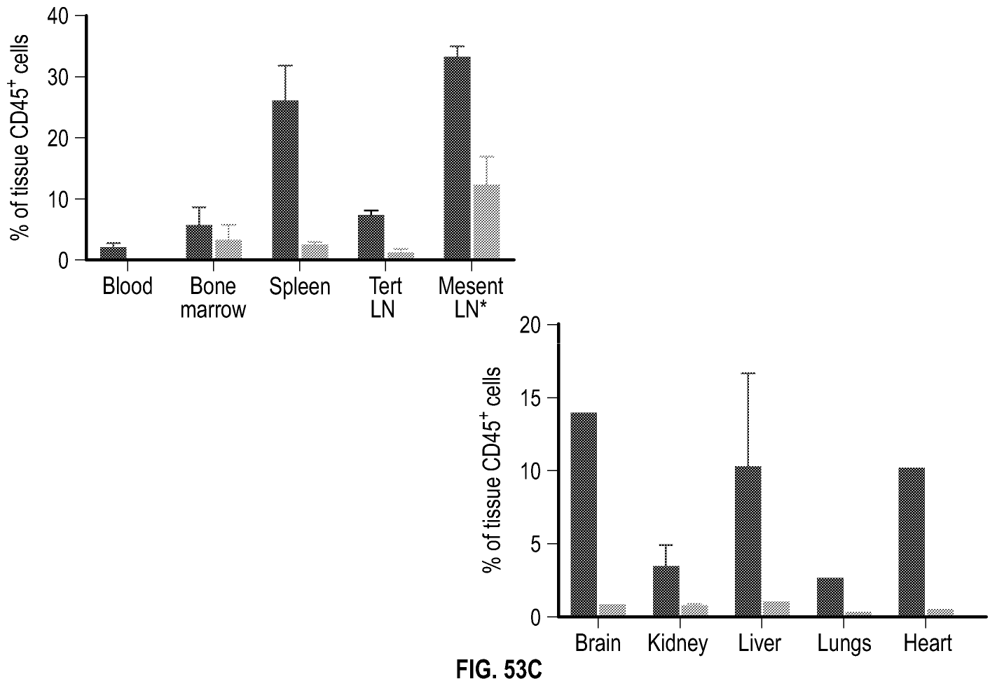

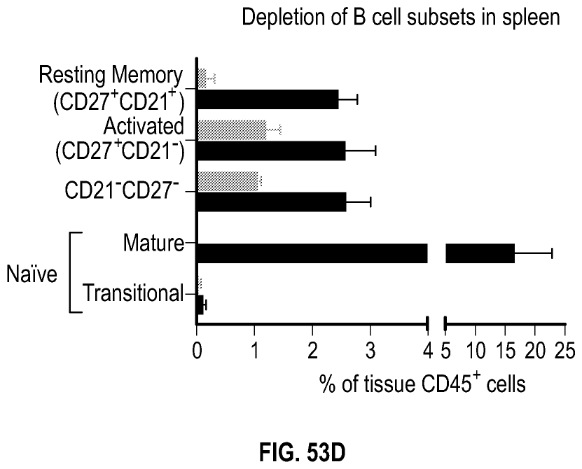

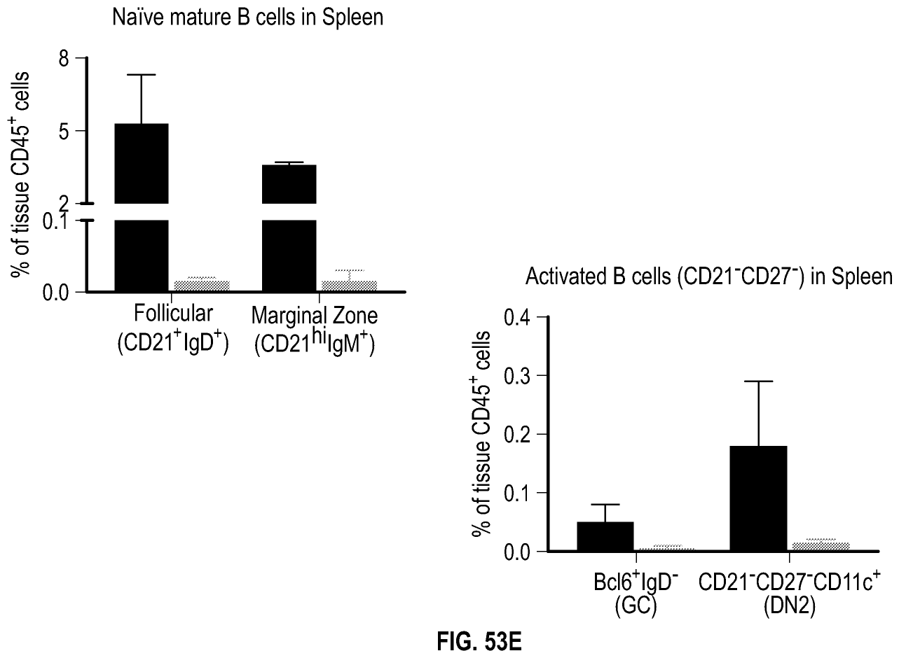

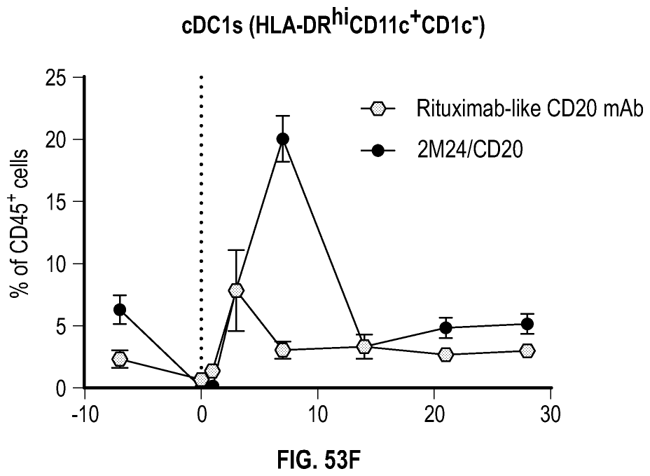

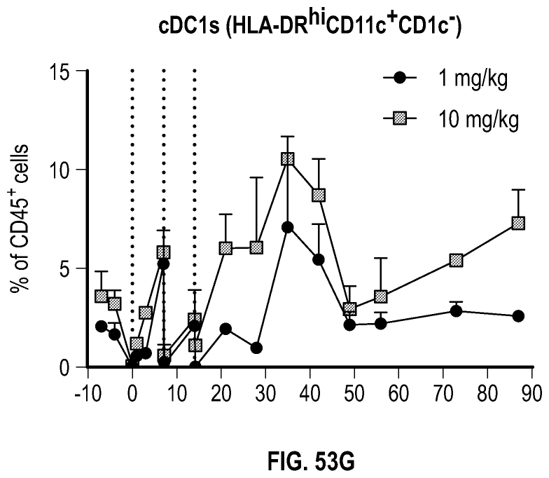

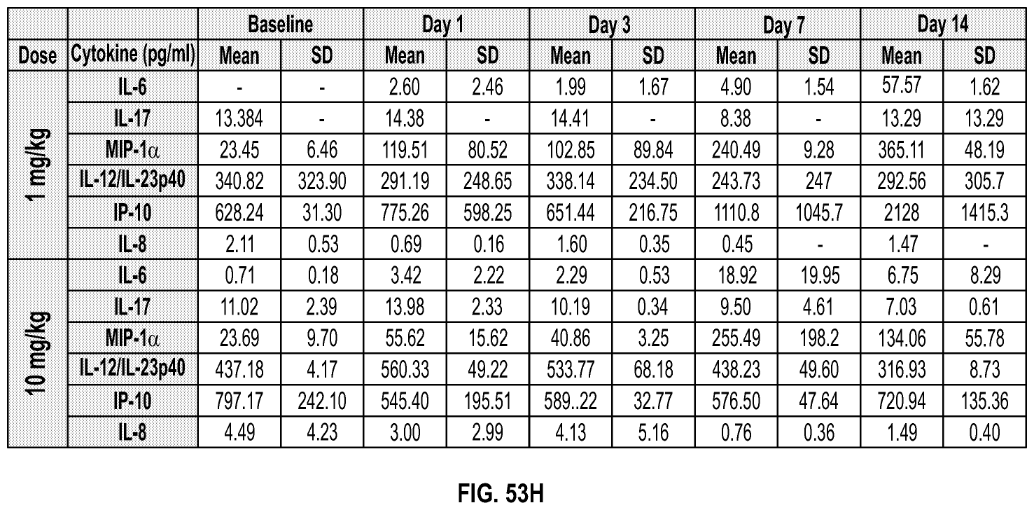

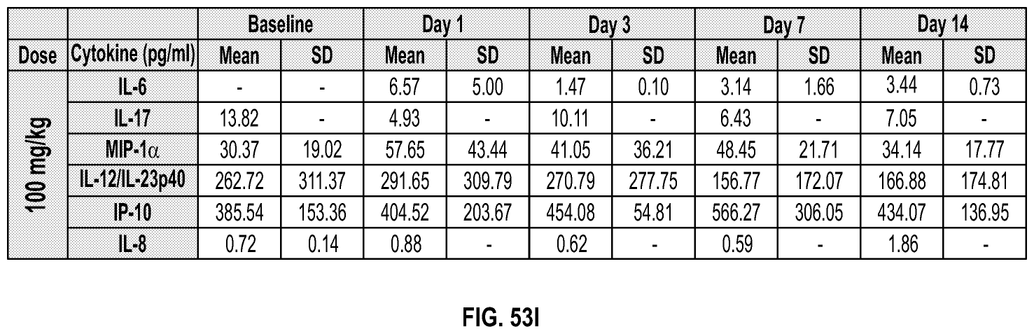

The results indicated that 2M24/CD20 bispecific binding protein induced robust B cell depletion in vivo in cynomolgus monkeys. All dose levels of 2M24/CD20 bispecific demonstrated robust B cell depletion in the peripheral blood of cynomolgus monkeys in vivo (FIG. 53A). 2M24/CD20 bispecific at 1 mg/kg dose level showed complete depletion of B cells following the first dose; peripheral B cell started to recover with B cell level reaching 15% of baseline value by the end of the third dose and reaching 75% of baseline by day 56. At 10 mg/kg dose level, 2M24/CD20 bispecific induced near complete depletion following the first dose and was sustained until the third dose at day 15. Following the third dose the B cell levels started to slowly recover to about 15% of baseline by day 56. Finally, the 100 mg/kg dose induced complete B cell depletion which was sustained through all three doses.

B cell levels were also analyzed in lymphoid organs (bone marrow and lymph nodes). Following the above dose regimen, samples were collected at several timepoints from lymph node and bone marrow from 1 and 10 mg/kg groups to assess the presence of B cells by flow cytometry, and depletion was evaluated compared to the baseline level of the B cells.

The results indicated that all dose levels of 2M24/CD20 bispecific induced robust B cell depletion in both bone marrow lymph nodes of cynomolgus monkeys in vivo. At the end of three weekly doses 2M24/CD20 bispecific at both 1 and 10 mg/kg dose showed ˜50% depletion in bone marrow and lymph node (FIG. 53B). 2M24/CD20 bispecific at 100 mg/kg dose showed ˜50% B cell depletion in the bone marrow, but close to 90% B cell depletion in the lymph node.

B cell levels were further analyzed in lymphoid and non-lymphoid tissues. Two groups of naïve cynomolgus monkeys (n=2; 1 male and 1 female in each group) were treated with three weekly doses of vehicle and 100 mg/kg of 2M24/CD20 bispecific. Following the dose regimen, at day 15, one after the third dose, animals were sacrificed to harvest several lymphoid and non-lymphoid organs to assess the depletion of tissue B cells by flow cytometry. B cells were marked by the double expression of HLA-DR and CD19.

The results indicated that 2M24/CD20 bispecific binding protein induced robust B cell depletion in the periphery and lymphoid organs. As shown in FIG. 53C, 2M24/CD20 bispecific at 100 mg/kg showed near complete depletion of B cells in the peripheral blood and secondary lymphoid organs. Compared to the vehicle treated controls, 2M24/CD20 bispecific showed approximately 90%, 85% and 74% depletion of all B cells in the spleens, tertiary lymph node and mesenteric lymph node; B cell depletion in the bone marrow was limited and restricted to mature B cell compartment (not shown). Within non-lymphoid organs, 2M24/CD20 bispecific at 100 mg/kg showed approximately 94%, 77%, 90%, 88%, and 95% B cell depletion in brain, kidney, liver, lungs and heart respectively, as compared to the vehicle treated cohorts. Together, these data indicate the robust efficacy of 2M24/CD20 bispecific binding protein at 100 mg/kg and its reach within deep niche of lymphoid as well as non-lymphoid organs in cynomolgus monkeys.

Depletion of B cell subsets in the spleens were also examined. Two groups of naïve cynomolgus monkeys (n=2; 1 male and 1 female in each group) were treated with three weekly doses of vehicle, and 100 mg/kg of 2M24/CD20 bispecific. Following the dose regimen, at day 15, one after the third dose, animals were sacrificed to harvest spleens to assess the extent of depletion of different B cell-subsets by flow cytometry. B cells were marked by the double expressions of HLA-DR and CD19.

The results indicated that 2M24/CD20 bispecific at 100 mg/kg induced robust, deep-tissue depletion of naïve and activated B cell subsets in the spleen of cynomolgus monkeys. 2M24/CD20 bispecific at 100 mg/kg showed near complete depletion of naïve B cells in the spleen. As compared to vehicle treated cohort, 2M24/CD20 bispecific showed 74% and 99% depletion of the transitional and mature naïve B cells respectively; transitional subsets are differentiated from mature by the expression of CD10 marker (FIGS. 53D & 53E). The complete depletion of splenic naïve B cells by 2M24/CD20 bispecific was reflected in both naïve follicular (CD21⁺IgD^{hi}), and marginal zone (CD21^{hi}IgM⁺) B cell compartments as well. As compared to vehicle treated groups, 2M24/CD20 bispecific induced ˜55% and 93% depletion within the activated B cell compartment, and the memory B cells; B cell depletion within activated B cell compartment included germinal center B cells, or pathogenic DN2 B cell population (marked by CD21⁻CD27⁺CD11c⁺).

Peripheral myeloid cell activation was also examined and compared with that induced by rituximab-like anti-cyno CD20 antibody. Two groups of naïve cynomolgus monkeys (n=2; 1 male and 1 female in each group) were treated with a single dose of 5 mg/kg of 2M24/CD20 bispecific antibody (Fab-Fab format), and rituximab-like anti-CD20 antibody. Following the dose regimen, samples were collected at several timepoints from peripheral blood to assess the myeloid cell dynamics in the peripheral blood. In another experiment, two groups of naïve cynomolgus monkeys (n=2; 1 male and 1 female in each group) were treated with three weekly doses of 1, and 10 mg/kg of 2M24/CD20 bispecific (scFv-Fab format). Following the dose regimen, samples were collected at several timepoints from peripheral blood from 1 and 10 mg/kg groups to assess the myeloid cell dynamics in the peripheral blood.

The results indicated that 2M24/CD20 bispecific demonstrated robust myeloid cell expansion in circulation. Single dose of 2M24/CD20 bispecific (Fab-Fab) antibody showed an expansion of myeloid cells (HLA-DR⁺CD19⁻) in the peripheral blood of cynomolgus monkey (not shown). Specifically, a subset of dendritic cells—classical dendritic cell 1 (cDC1, characterized by HLA-DR^{hi}CD11c⁺CD1c⁻) showed prominent expansion in the circulation for both the doses, with the cDC1 cells peaking at day 10 from treatment initiation; the expansion of the myeloid cells and cDC1 population was not observed in rituximab-like CD20-mAb treated cohort (FIG. 53F). Similarly, three doses of 2M24/CD20 bispecific (scFv-Fab) at both 1 and 10 mg/kg showed near a prominent expansion of myeloid cells including cDC1 cell population; cDC1 cells peaked at day 40 from treatment initiation (FIG. 53G). Together these data support the 2M24/CD20 bispecific (both Fab-Fab and scFv-Fab format) mediated myeloid cell stimulation and active expansion of dendritic cell population cDC1 in the peripheral blood in cynomolgus monkeys.

Levels of pro-inflammatory cytokines in serum were also analyzed. Two groups of naïve cynomolgus monkeys (n=2; 1 male and 1 female in each group) were treated with three weekly doses of 1 and 10 mg/kg of 2M24/CD20 bispecific. Following the dose regimen, serum samples were collected at several timepoints from peripheral blood to assess the pro-inflammatory cytokine response by MSD cytokine array.

The results indicated that 2M24/CD20 bispecific at both doses showed moderate pro-inflammatory cytokine response in cynomolgus monkeys. As shown in FIG. 53H, the cytokine response was moderate at both 1 and 10 mg/kg dose of 2M24/CD20 bispecific with all 6 cytokines showing relatively stable levels from baseline through to day 14. Specifically, a transient increase in MIP-1a was noted following both 1 and 10 mg/kg of 2M24/CD20 bispecific treatment. However, no sustained increases in any of the pro-inflammatory cytokine was noted. No detectable level of IL-1b and TNF-α was noted at the baseline or following the 2M24/CD20 bispecific treatment (not shown).

Two groups of naïve cynomolgus monkeys (n=2; 1 male and 1 female in each group) were also treated with three weekly doses of 1 and 100 mg/kg of 2M24/CD20 bispecific, and cytokines in serum were analyzed as described above. As shown in FIG. 53I, the cytokine response was moderate at the 100 mg/kg dose with all 6 cytokines showing relatively stable levels from baseline through to day 14. IP-10 levels seem most affected rising with levels consistently greater than baseline at all timepoints, and a peak of ˜560 pg/ml on day 7. MIP-1α levels also show a consistent increase from baseline at all timepoints, with a high of ˜57 pg/ml at day 1. No detectable level of IL-1b and TNF-a was noted at the baseline or following the 2M24/CD20 bispecific treatment (not shown).

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in the entirety by reference.

What is claimed is:

1. A multispecific binding protein with a first antigen binding domain that binds to human Dectin-1 and a second antigen binding domain that binds to human CD20, comprising:
a first polypeptide chain comprising the amino
acid sequence
(SEQ ID NO: 31)
QVQLVQSGAEVKKPGASVKVSCKSSGYTFTDYYIHWVRQAPGQGLEWMGW
INPNSGDTNYAQKFQGRITMTRDTSISTAYLELSRLRSDDTAVFYCARNS
GSYSFGYWGQGTLVTVSSGGGGSGGGGSGGGGSGGGGSDIQMTQSPSSVS
ASVGDRVTITCRASQGISSWLAWYQQKPGKAPKLLIFGASSLQSGVPSRF
SGSGSGTDFTLTVSSLQPEDFATYYCQQAYSFPFTFGPGTKVDIEEPKRS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLWCLVK
GFYPSDIAVEWESNGQPENNYKTTPPVLDSDGSFFLYSKLTVDKSRWQQG
NVFSCSVMHEALHNHYTQKSLSLSPG
or
(SEQ ID NO: 35)
QVQLVQSGAEVKKPGASVKVSCKSSGYTFTDYYIHWVRQAPGQGLEWMGW
INPNSGDTNYAQKFQGRITMTRDTSISTAYLELSRLRSDDTAVFYCARNS
GSYSFGYWGQGTLVTVSSGGGGGGGGSGGGGSGGGGSDIQMTQSPSSVSA
SVGDRVTITCRASQGISSWLAWYQQKPGKAPKLLIFGASSLQSGVPSRFS
GSGSGTDFTLTVSSLQPEDFATYYCQQAYSFPFTFGPGTKVDIEEPKRSD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLWCLVKG
FYPSDIAVEWESNGQPENNYKTTPPVLDSDGSFFLYSKLTVDKSRWQQGN
VFSCSVMHEALHNHYTQKSLSLSPGK;
a second polypeptide chain comprising the amino
acid sequence
(SEQ ID NO: 32)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLSCAVKGFYPSDIAVEWESNGQPENNYKTTPP
VLDSDGSFFLVSKLTVDKSRWQQGNVFSCSVMHEALHNHYTQKSLSLSPG
or
(SEQ ID NO: 36)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLSCAVKGFYPSDIAVEWESNGQPENNYKTTPP
VLDSDGSFFLVSKLTVDKSRWQQGNVFSCSVMHEALHNHYTQKSLSLSPG
K;
and
a third polypeptide chain comprising the amino
acid sequence
(SEQ ID NO: 33)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

2. The multispecific binding protein of claim 1, wherein the first polypeptide chain comprises the amino acid sequence of SEQ ID NO:31, the second polypeptide chain comprises the amino acid sequence of SEQ ID NO:32, and the third polypeptide chain comprises the amino acid sequence of SEQ ID NO:33.

3. The multispecific binding protein of claim 1, wherein the first polypeptide chain comprises the amino acid sequence of SEQ ID NO:35, the second polypeptide chain comprises the amino acid sequence of SEQ ID NO:36, and the third polypeptide chain comprises the amino acid sequence of SEQ ID NO:33.

4. A multispecific binding protein with a first antigen binding domain that binds to human Dectin-1 and a second antigen binding domain that binds to human CD20, comprising:
a first polypeptide chain comprising the amino
acid sequence
(SEQ ID NO: 37)
QVQLVQSGAEVKKPGASVKVSCKSSGYTFTDYYIHWVRQAPGQGLEWMGW
INPNSGDTNYAQKFQGRITMTRDTSISTAYLELSRLRSDDTAVFYCARNS
GSYSFGYWGQGTLVTVSSGGGGSGGGGSGGGGSGGGGSDIQMTQSPSSVS
ASVGDRVTITCRASQGISSWLAWYQQKPGKAPKLLIFGASSLQSGVPSRF
SGSGSGTDFTLTVSSLQPEDFATYYCQQAYSFPFTFGPGTKVDIEEPKRS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLSCAVK
GFYPSDIAVEWESNGQPENNYKTTPPVLDSDGSFFLVSKLTVDKSRWQQG
NVFSCSVMHEALHNHYTQKSLSLSPG
or
(SEQ ID NO: 39)
QVQLVQSGAEVKKPGASVKVSCKSSGYTFTDYYIHWVRQAPGQGLEWMGW
INPNSGDTNYAQKFQGRITMTRDTSISTAYLELSRLRSDDTAVFYCARNS
GSYSFGYWGQGTLVTVSSGGGGSGGGGSGGGGSGGGGSDIQMTQSPSSVS
ASVGDRVTITCRASQGISSWLAWYQQKPGKAPKLLIFGASSLQSGVPSRF
SGSGSGTDFTLTVSSLQPEDFATYYCQQAYSFPFTFGPGTKVDIEEPKRS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLSCAVK
GFYPSDIAVEWESNGQPENNYKTTPPVLDSDGSFFLVSKLTVDKSRWQQG
NVFSCSVMHEALHNHYTQKSLSLSPGK;
a second polypeptide chain comprising the amino
acid sequence
(SEQ ID NO: 38)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLWCLVKGFYPSDIAVEWESNGQPENNYKTTPP
VLDSDGSFFLYSKLTVDKSRWQQGNVFSCSVMHEALHNHYTQKSLSLSPG
or
(SEQ ID NO: 40)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
DPKTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLWCLVKGFYPSDIAVEWESNGQPENNYKTTPP
VLDSDGSFFLYSKLTVDKSRWQQGNVFSCSVMHEALHNHYTQKSLSLSPG
K;
and
a third polypeptide chain comprising the amino
acid sequence
(SEQ ID NO: 33)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

5. The multispecific binding protein of claim 4, wherein the first polypeptide chain comprises the amino acid sequence of SEQ ID NO:37, the second polypeptide chain comprises the amino acid sequence of SEQ ID NO:38, and the third polypeptide chain comprises the amino acid sequence of SEQ ID NO:33.

6. The multispecific binding protein of claim 4, wherein the first polypeptide chain comprises the amino acid sequence of SEQ ID NO:39, the second polypeptide chain comprises the amino acid sequence of SEQ ID NO:40, and the third polypeptide chain comprises the amino acid sequence of SEQ ID NO:33.

7. The multispecific binding protein of any one of claims 1-6, wherein at least one of the first and second polypeptide chains is/are non-fucosylated.

8. The multispecific binding protein of claim 7, wherein both of the first and second polypeptide chains are non-fucosylated.

9. The multispecific binding protein of any one of claims 1-8, wherein the first antigen-binding domain:
(a) binds to human Dectin-1 expressed on the surface of a macrophage, monocyte, dendritic cell, or granulocyte;
(b) binds to human Dectin-1 expressed on the surface of a cell with an EC50 of less than 2 nM;
(c) is capable of binding human or cynomolgus Dectin-1; and/or
(d) does not compete with a native ligand of human Dectin-1.

10. The multispecific binding protein of any one of claims 1-9, wherein the second antigen binding domain binds to human CD20 expressed on the surface of a B cell.

11. A polynucleotide encoding the multispecific binding protein of any one of claims 1-10.

12. A vector comprising the polynucleotide of claim 11.

13. An isolated host cell comprising the polynucleotide of claim 11 or the vector of claim 12.

14. The isolated host cell of claim 13, wherein the host cell is a yeast, insect, plant, or prokaryotic cell.

15. The isolated host cell of claim 13, wherein the host cell is a mammalian cell.

16. The isolated host cell of claim 15, wherein the mammalian cell is a Chinese hamster ovary (CHO) cell.

17. The isolated host cell of claim 15 or claim 16, wherein the host cell comprises an alpha1,6-fucosyltransferase (Fut8) or alpha-1,3-mannosyl-glycoprotein 2-beta-N-acetylglucosaminyltranferase (MGAT1) knockout.

18. The isolated host cell of claim 15 or claim 16, wherein the host cell overexpresses β1,4-N-acetylglucosaminyltransferase III (GnT-III).

19. The isolated host cell of claim 18, wherein the host cell further overexpresses Golgi μ-mannosidase II (ManII).

20. A method of producing an antibody or multispecific binding protein, comprising culturing the host cell of any one of claims 13-19 under conditions suitable for production of the multispecific binding protein.

21. The method of claim 20, further comprising recovering the antibody or multispecific binding protein.

22. The method of claim 20 or claim 21, wherein, prior to production of the antibody or multispecific binding protein, the host cell is treated with kifunensine.

23. A multispecific binding protein produced by the method of any one of claims 20-22.

24. A pharmaceutical composition comprising the multispecific binding protein of any one of claims 1-10 and 23 and a pharmaceutically acceptable carrier.

25. The composition of claim 24, wherein the composition comprises a mixture of multispecific binding protein species, wherein each species comprises a first polypeptide chain that comprises the amino acid sequence of SEQ ID NO:31 or SEQ ID NO:35, a second polypeptide chain that comprises the amino acid sequence of SEQ ID NO:32 or SEQ ID NO:36, and a third polypeptide chain that comprises the amino acid sequence of SEQ ID NO:33.

26. The composition of claim 24, wherein the first polypeptide chain comprises the amino acid sequence of SEQ ID NO:31, the second polypeptide chain comprises the amino acid sequence of SEQ ID NO:32, and the third polypeptide chain comprises the amino acid sequence of SEQ ID NO:33.

27. The composition of claim 24, wherein the first polypeptide chain comprises the amino acid sequence of SEQ ID NO:35, the second polypeptide chain comprises the amino acid sequence of SEQ ID NO:36, and the third polypeptide chain comprises the amino acid sequence of SEQ ID NO:33.

28. The composition of claim 24, wherein the composition comprises a mixture of multispecific binding protein species, wherein each species comprises a first polypeptide chain that comprises the amino acid sequence of SEQ ID NO:37 or SEQ ID NO:39, a second polypeptide chain that comprises the amino acid sequence of SEQ ID NO:38 or SEQ ID NO:40, and a third polypeptide chain that comprises the amino acid sequence of SEQ ID NO:33.

29. The composition of claim 24, wherein the first polypeptide chain comprises the amino acid sequence of SEQ ID NO:37, the second polypeptide chain comprises the amino acid sequence of SEQ ID NO:38, and the third polypeptide chain comprises the amino acid sequence of SEQ ID NO:33.

30. The composition of claim 24, wherein the first polypeptide chain comprises the amino acid sequence of SEQ ID NO:39, the second polypeptide chain comprises the amino acid sequence of SEQ ID NO:40, and the third polypeptide chain comprises the amino acid sequence of SEQ ID NO:33.

31. A method of treating a disease or disorder, comprising administering an effective amount of the multispecific binding protein of any one of claims 1-10 and 23 or the composition of any one of claims 24-30 to an individual in need thereof.

32. The method of claim 31, wherein the individual has a B cell-mediated disease or disorder.

33. The method of claim 32, wherein the B cell-mediated disease or disorder is cancer.

34. The method of claim 33, wherein the cancer is non-Hodgkin's lymphoma or chronic lymphocytic leukemia.

35. The method of claim 32, wherein the B cell-mediated disease or disorder is an autoimmune disease or disorder.

36. The method of claim 35, wherein the autoimmune disease or disorder is rheumatoid arthritis, systemic lupus erythematosus (SLE), multiple sclerosis, and Wegener's granulomatosis.

37. The method of any one of claims 31-36, wherein the individual is a human.
